SEQUENCE LISTING (333 sequences)

SEQ ID NO: 1 - protein, 117 aa, synthetic construct
  REGION 1..117: note = VH1.8/VL1.6 heavy chain variable (VH) region
QVQLVQSGAEVKKPGASVKVSCKASGYTFTNYAMHWVRQAPGQRLEWMGTIYPGNDDTKYNQKFQGRVTITADTSASTAYMELSSLRSEDTAVYYCARGGFRAMDYWGQGTLVTVSS

SEQ ID NO: 2 - protein, 112 aa, synthetic construct
  REGION 1..112: note = VH1.8/VL1.6 light chain variable (VL) region
DIVMTQSPLSLPVTPGEPASISCRSSQSLLHSNANTYLGWYLQKPGQSPQLLIYKGSNRFSGVPDRFSGSGSGTDFTLKISRVEAEDVGVYYCFQASHVPYTFGQGTKLEIK

SEQ ID NO: 3 - protein, 117 aa, synthetic construct; identical to SEQ ID NO: 1
  REGION 1..117: note = VH1.8/VL1.6-DI heavy chain variable (VH) region

SEQ ID NO: 4 - protein, 112 aa, synthetic construct
  REGION 1..112: note = VH1.8/VL1.6-DI light chain variable (VL) region
DIVMTQSPLSLPVTPGEPASISCRSSQSLLHSNANTYLGWYLQKPGQSPQLAIYKGSNRFSGVPDRFSGSGSGTDFTLKISRVEAEDAGVYYCFQASHVPYTFGQGTKLEIK

SEQ ID NO: 5 - protein, 117 aa, synthetic construct; identical to SEQ ID NO: 1
  REGION 1..117: note = VH1.8/VL1.1 heavy chain variable (VH) region

SEQ ID NO: 6 - protein, 112 aa, synthetic construct
  REGION 1..112: note = VH1.8/VL1.1 light chain variable (VL) region
DIVMTQSPLSLPVTPGEPASISCRSSQSLLHSNGNTYLGWYLQKPGQSPQLLIYKGSNRFSGVPDRFSGSGSGTDFTLKISRVEAEDVGVYYCFQASHVPYTFGQGTKLEIK

SEQ ID NO: 7 - protein, 117 aa, synthetic construct; identical to SEQ ID NO: 1
  REGION 1..117: note = VH1.8/VL1.5 heavy chain variable (VH) region

SEQ ID NO: 8 - protein, 112 aa, synthetic construct
  REGION 1..112: note = VH1.8/VL1.5 light chain variable (VL) region
DIVMTQSPLSLPVTPGEPASISCRSSQSLLHSNAYNYLGWYLQKPGQSPQLLIYKGSNRFSGVPDRFSGSGSGTDFTLKISRVEAEDVGVYYCFQASHVPYTFGQGTKLEIK

SEQ ID NO: 9 - protein, 117 aa, synthetic construct; identical to SEQ ID NO: 1
  REGION 1..117: note = VH1.8/VL1.4 heavy chain variable (VH) region

SEQ ID NO: 10 - protein, 112 aa, synthetic construct
  REGION 1..112: note = VH1.8/VL1.4 light chain variable (VL) region
DIVMTQSPLSLPVTPGEPASISCRSSQSLLHSNAYNYLGWYLQKPGQSPQLLIYKGSNRFSGVPDRFSGSGSGTDFTLKISRVEAEDVGVYYCFQASQVPYTFGQGTKLEIK

SEQ ID NO: 11 - protein, 117 aa, synthetic construct; identical to SEQ ID NO: 1
  REGION 1..117: note = VH1.8/VL1.3 heavy chain variable (VH) region

SEQ ID NO: 12 - protein, 112 aa, synthetic construct
  REGION 1..112: note = VH1.8/VL1.3 light chain variable (VL) region
DIVMTQSPLSLPVTPGEPASISCRSSQSLLHSNANTYLGWYLQKPGQSPQLLIYKGSNRFSGVPDRFSGSGSGTDFTLKISRVEAEDVGVYYCFQASQVPYTFGQGTKLEIK

SEQ ID NO: 13 - protein, 117 aa, synthetic construct; identical to SEQ ID NO: 1
  REGION 1..117: note = VH1.8/VL1.2 heavy chain variable (VH) region

SEQ ID NO: 14 - protein, 112 aa, synthetic construct
  REGION 1..112: note = VH1.8/VL1.2 light chain variable (VL) region
DIVMTQSPLSLPVTPGEPASISCRSSQSLLHSNGNTYLGWYLQKPGQSPQLLIYKGSNRFSGVPDRFSGSGSGTDFTLKISRVEAEDVGVYYCFQASQVPYTFGQGTKLEIK

SEQ ID NO: 15 - protein, 117 aa, synthetic construct
  REGION 1..117: note = D6 heavy chain variable (VH) region
EVQLVQSGAEVKKPGESLKISCKGSGYSFTNYNIHWVRQMPGKGLEWMGTIYPGNSDTSYNPSFQGQVTISADKSISTAYLQWSSLKASDTAMYYCARGGVRAMDYWGQGTLVTVSS

SEQ ID NO: 16 - protein, 112 aa, synthetic construct
  REGION 1..112: note = D6 light chain variable (VL) region
DIVMTQSPLSLPVTPGEPASISCRSSQSLLHSNGNTYLGWYLQKPGQSPQLLIYKGSNRFSGVPDRFSGSGSGTDFTLKISRVEAEDVGVYYCMQASQVPYTFGQGTKLEIK

SEQ ID NO: 17 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 330 aa, Homo sapiens [sequence omitted]

SEQ ID NO: 20 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 22 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 25 - protein, 323 aa, Macaca fascicularis
MWPLVAALLLGSACCGSAQLLFNKTKSVEFTFCNDTVVIPCFVTNMEAQNTTEVYVKWKFKGRDIYTFDGALNKSTAPANFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLMITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 26 - protein, 5 aa, Homo sapiens
RDELT

SEQ ID NO: 27 - protein, 4 aa, Homo sapiens
REEM

SEQ ID NO: 28 - protein, 10 aa, synthetic construct
  REGION 1..10: note = anti-CD47 antibody molecule or antigen-binding portion HCDR1
  SITE 6: note = misc_feature - Xaa can be Asn or a conservative substitution of Asn
  SITE 8: note = misc_feature - Xaa can be Asn or a conservative substitution of Asn
  SITE 9: note = misc_feature - Xaa can be Met or a conservative substitution of Met
  SITE 10: note = misc_feature - Xaa can be His or any amino acid
GYSFTXYXXX

SEQ ID NO: 29 - protein, 19 aa, synthetic construct
  REGION 1..19: note = anti-CD47 antibody molecule or antigen-binding portion HCDR2
  SITE 1: note = misc_feature - Xaa can be Met or a conservative substitution of Met
  SITE 3: note = misc_feature - Xaa can be Thr or any amino acid
  SITE 8: note = misc_feature - Xaa can be Asn or any amino acid
  SITE 9: note = misc_feature - Xaa can be Asp or any amino acid
  SITE 12: note = misc_feature - Xaa can be Ser or any amino acid
  SITE 14: note = misc_feature - Xaa can be Asn or any amino acid
  SITE 15: note = misc_feature - Xaa can be Gln or any amino acid
  SITE 16: note = misc_feature - Xaa can be Lys or any amino acid
  SITE 18: note = misc_feature - Xaa can be Gln or Lys
  SITE 19: note = misc_feature - Xaa can be Gly or Asp
XGXIYPGXXDTXYXXXFXX

SEQ ID NO: 30 - protein, 8 aa, synthetic construct
  REGION 1..8: note = anti-CD47 antibody molecule or antigen-binding portion HCDR3
  SITE 1: note = misc_feature - Xaa can be Gly or any amino acid
  SITE 3: note = misc_feature - Xaa can be Tyr or any amino acid
  SITE 5: note = misc_feature - Xaa can be Ala or any amino acid
  SITE 6: note = misc_feature - Xaa can be Met or any amino acid
XGXRXXDY

SEQ ID NO: 31 - protein, 10 aa, synthetic construct
  REGION 1..10: note = anti-CD47 antibody molecule or antigen-binding portion HCDR1
  SITE 6: note = misc_feature - Xaa can be Asn or any amino acid
  SITE 8: note = misc_feature - Xaa can be Asn or any amino acid
GYTFTXYXMH

SEQ ID NO: 32 - protein, 19 aa, synthetic construct
  REGION 1..19: note = anti-CD47 antibody molecule or antigen-binding portion HCDR2
  SITE 10: note = misc_feature - Xaa can be Asp or any amino acid
  SITE 12: note = misc_feature - Xaa can be Ser or any amino acid
  SITE 14: note = misc_feature - Xaa can be Asn or any amino acid
MGTIYPGNDXTXYXQKFQG

SEQ ID NO: 33 - protein, 10 aa, synthetic construct
  REGION 1..10: note = 5F9G4 murine/humanized antibody HCDR1
GYTFTNYNMH

SEQ ID NO: 34 - protein, 19 aa, synthetic construct
  REGION 1..19: note = 5F9G4 murine/humanized antibody HCDR2
MGTIYPGNDDTSYNQKFKD

SEQ ID NO: 35 - protein, 8 aa, synthetic construct
  REGION 1..8: note = 5F9G4 murine/humanized antibody HCDR3
GGYRAMDY

SEQ ID NO: 36 - protein, 16 aa, synthetic construct
  REGION 1..16: note = anti-CD47 antibody molecule or antigen-binding portion LCDR1
  SITE 6: note = misc_feature - Xaa can be Ile or a conservative substitution of Ile
  SITE 7: note = misc_feature - Xaa can be Val or a conservative substitution of Val
  SITE 8: note = misc_feature - Xaa can be Tyr or a conservative substitution of Tyr
  SITE 10: note = misc_feature - Xaa can be Asn or any amino acid
  SITE 11: note = misc_feature - Xaa can be Gly or any amino acid
  SITE 12: note = misc_feature - Xaa can be Asn or any amino acid
  SITE 13: note = misc_feature - Xaa can be Thr or any amino acid
  SITE 16: note = misc_feature - Xaa can be Gly or any amino acid
RSSQSXXXSXXXXYLX

SEQ ID NO: 37 - protein, 7 aa, synthetic construct
  REGION 1..7: note = anti-CD47 antibody molecule or antigen-binding portion LCDR2
  SITE 2: note = misc_feature - Xaa can be Val or any amino acid
  SITE 6: note = misc_feature - Xaa can be Phe or any amino acid
KXSNRXS

SEQ ID NO: 38 - skipped

SEQ ID NO: 39 - protein, 16 aa, synthetic construct
  REGION 1..16: note = 5F9G4 murine/humanized antibody LCDR1
RSSQSIVYSNGNTYLG

SEQ ID NO: 40 - protein, 7 aa, synthetic construct
  REGION 1..7: note = 5F9G4 murine/humanized antibody LCDR2
KVSNRFS

SEQ ID NO: 41 - protein, 9 aa, synthetic construct
  REGION 1..9: note = 5F9G4 murine/humanized antibody LCDR3
FQGSHVPYT

SEQ ID NO: 42 - protein, 10 aa, synthetic construct
  REGION 1..10: note = anti-CD47 antibody molecule or antigen-binding portion HCDR1
  SITE 3: note = misc_feature - Xaa can be Ser or a conservative substitution of Ser
  SITE 6: note = misc_feature - Xaa can be Asn or a conservative substitution of Asn
  SITE 8: note = misc_feature - Xaa can be Asn or any other amino acid
  SITE 9: note = misc_feature - Xaa can be Met or a conservative substitution of Met
  SITE 10: note = misc_feature - Xaa can be His or any other amino acid
GYXFTXYXXX

SEQ ID NO: 43 - protein, 19 aa, synthetic construct
  REGION 1..19: note = anti-CD47 antibody molecule or antigen-binding portion HCDR2
  SITE 1: note = misc_feature - Xaa can be Met or a conservative substitution of Met
  SITE 2: note = misc_feature - Xaa can be Gly or a conservative substitution of Gly
  SITE 3: note = misc_feature - Xaa can be Thr or any other amino acid
  SITE 8: note = misc_feature - Xaa can be Asn or a conservative substitution of Asn
  SITE 9: note = misc_feature - Xaa can be Asp or any other amino acid
  SITE 10: note = misc_feature - Xaa can be Asp or any other amino acid
  SITE 12: note = misc_feature - Xaa can be Ser or any other amino acid
  SITE 14: note = misc_feature - Xaa can be Asn or any other amino acid
  SITE 15: note = misc_feature - Xaa can be Gln or any other amino acid
  SITE 16: note = misc_feature - Xaa can be Lys or any other amino acid
XXXIYPGXXXTXYXXXFQG

SEQ ID NO: 44 - protein, 8 aa, synthetic construct
  REGION 1..8: note = anti-CD47 antibody molecule or antigen-binding portion HCDR3
  SITE 1: note = misc_feature - Xaa can be Gly or any other amino acid
  SITE 3: note = misc_feature - Xaa can be Tyr or any other amino acid
  SITE 5: note = misc_feature - Xaa can be Ala or any other amino acid
  SITE 6: note = misc_feature - Xaa can be Met or any other amino acid
XGXRXXDY

SEQ ID NO: 45 - protein, 16 aa, synthetic construct
  REGION 1..16: note = anti-CD47 antibody molecule or antigen-binding portion LCDR1
  SITE 6: note = misc_feature - Xaa can be Ile or a conservative substitution of Ile
  SITE 7: note = misc_feature - Xaa can be Val or a conservative substitution of Val
  SITE 8: note = misc_feature - Xaa can be Tyr or a conservative substitution of Tyr
  SITE 10: note = misc_feature - Xaa can be Asn or any other amino acid
  SITE 11: note = misc_feature - Xaa can be Gly or any other amino acid
  SITE 12: note = misc_feature - Xaa can be Asn or any other amino acid
  SITE 13: note = misc_feature - Xaa can be Thr or any other amino acid
  SITE 16: note = misc_feature - Xaa can be Gly or any other amino acid
RSSQSXXXSXXXXYLX

SEQ ID NO: 46 - protein, 7 aa, synthetic construct
  REGION 1..7: note = anti-CD47 antibody molecule or antigen-binding portion LCDR2
  SITE 2: note = misc_feature - Xaa can be Val or any other amino acid
  SITE 6: note = misc_feature - Xaa can be Phe or any other amino acid
KXSNRXS

SEQ ID NO: 47 - skipped

SEQ ID NO: 48 - protein, 10 aa, synthetic construct
  REGION 1..10: note = anti-CD47 antibody molecule or antigen-binding portion HCDR1
GYTFTNYAMH

SEQ ID NO: 49 - protein, 19 aa, synthetic construct
  REGION 1..19: note = anti-CD47 antibody molecule or antigen-binding portion HCDR2
MGTIYPGNDDTKYNQKFQG

SEQ ID NO: 50 - protein, 8 aa, synthetic construct
  REGION 1..8: note = anti-CD47 antibody molecule or antigen-binding portion HCDR3
GGFRAMDY

SEQ ID NO: 51 - protein, 16 aa, synthetic construct
  REGION 1..16: note = anti-CD47 antibody molecule or antigen-binding portion LCDR1
RSSQSLLHSNGNTYLG

SEQ ID NO: 52 - protein, 7 aa, synthetic construct
  REGION 1..7: note = anti-CD47 antibody molecule or antigen-binding portion LCDR2
KGSNRFS

SEQ ID NO: 53 - protein, 9 aa, synthetic construct
  REGION 1..9: note = anti-CD47 antibody molecule or antigen-binding portion LCDR3
FQASHVPYT

SEQ ID NO: 54 - protein, 9 aa, synthetic construct
  REGION 1..9: note = anti-CD47 antibody molecule or antigen-binding portion LCDR3
FQASQVPYT

SEQ ID NO: 55 - protein, 16 aa, synthetic construct
  REGION 1..16: note = anti-CD47 antibody molecule or antigen-binding portion LCDR1
RSSQSLLHSNANTYLG

SEQ ID NO: 56 - protein, 16 aa, synthetic construct
  REGION 1..16: note = anti-CD47 antibody molecule or antigen-binding portion LCDR1
RSSQSLLHSNAYNYLG

SEQ ID NO: 57 - protein, 10 aa, synthetic construct
  REGION 1..10: note = anti-CD47 antibody molecule or antigen-binding portion HCDR1
GYSFTNYNIH

SEQ ID NO: 58 - protein, 19 aa, synthetic construct
  REGION 1..19: note = anti-CD47 antibody molecule or antigen-binding portion HCDR2
MGTIYPGDSDTRYNPKFQG

SEQ ID NO: 59 - protein, 8 aa, synthetic construct
  REGION 1..8: note = anti-CD47 antibody molecule or antigen-binding portion HCDR3
GGYRAEDY

SEQ ID NO: 60 - protein, 8 aa, synthetic construct
  REGION 1..8: note = anti-CD47 antibody molecule or antigen-binding portion LCDR3
MQGSHVPY

SEQ ID NO: 61 - protein, 19 aa, synthetic construct
  REGION 1..19: note = anti-CD47 antibody molecule or antigen-binding portion HCDR2
MGTIYPGNSDTSYNPSFQG

SEQ ID NO: 62 - protein, 8 aa, synthetic construct
  REGION 1..8: note = anti-CD47 antibody molecule or antigen-binding portion HCDR3
GGVRAMDY

SEQ ID NO: 63 - protein, 9 aa, synthetic construct
  REGION 1..9: note = anti-CD47 antibody molecule or antigen-binding portion LCDR3
MQASQVPYT

SEQ ID NO: 64 - protein, 10 aa, synthetic construct
  REGION 1..10: note = anti-CD47 antibody molecule or antigen-binding portion VH CDR
GYSFTNYNMH

SEQ ID NO: 65 - protein, 19 aa, synthetic construct
  REGION 1..19: note = anti-CD47 antibody molecule or antigen-binding portion VH CDR
MGTIYPGNDDTSYNQKFQG

SEQ ID NO: 66 - protein, 8 aa, synthetic construct
  REGION 1..8: note = anti-CD47 antibody molecule or antigen-binding portion VH CDR
GGYRAMDY

SEQ ID NO: 67 - protein, 16 aa, synthetic construct
  REGION 1..16: note = anti-CD47 antibody molecule or antigen-binding portion VL CDR
RSSGSIVYSNGNTYLG

SEQ ID NO: 68 - protein, 7 aa, synthetic construct
  REGION 1..7: note = anti-CD47 antibody molecule or antigen-binding portion VL CDR
KVSNRFS

SEQ ID NO: 69 - protein, 9 aa, synthetic construct
  REGION 1..9: note = anti-CD47 antibody molecule or antigen-binding portion VL CDR
FQGSHVPYT

SEQ ID NO: 70 - protein, 40 aa, synthetic construct
  REGION 1..40: note = anti-CD47 antibody molecule or antigen-binding portion LCDR1
DIVMTQSPLSLPVTPGEPASISCRSSQSIVYSNGNTYLGW

SEQ ID NO: 71 - protein, 40 aa, synthetic construct
  REGION 1..40: note = anti-CD47 antibody molecule or antigen-binding portion HCDR1
QVQLVQSGAEVKKPGASVKVSCKASGYTFTNYNMHWVRQA

SEQ ID NO: 72 - protein, 37 aa, synthetic construct
  REGION 1..37: note = anti-CD47 antibody molecule or antigen-binding portion HCDR3
MELSSLRSEDTAVYYCARGGYRAMDYWGQGTLVTVSS

SEQ ID NO: 73 - protein, 77 aa, synthetic construct
  REGION 1..77: note = anti-CD47 antibody molecule or antigen-binding portion LCDR2
DIVMTQSPLSLPVTPGEPASISCRSSQSLLHSNGNTYLGWYLQKPGQSPQLLIYKGSNRFSGVPDRFSGSGSGTDFT

SEQ ID NO: 74 - protein, 35 aa, synthetic construct
  REGION 1..35: note = anti-CD47 antibody molecule or antigen-binding portion LCDR3
LKISRVEAEDVGVYYCFQASHVPYTFGQGTKLEIK

SEQ ID NO: 75 - protein, 19 aa, synthetic construct
  REGION 1..19: note = anti-CD47 antibody molecule or antigen-binding portion HCDR2
  SITE 1: note = misc_feature - Xaa can be Met or a conservative substitution of Met
  SITE 2: note = misc_feature - Xaa can be Gly or a conservative substitution of Gly
  SITE 3: note = misc_feature - Xaa can be Thr or any other amino acid
  SITE 8: note = misc_feature - Xaa can be Asn or any other amino acid
  SITE 9: note = misc_feature - Xaa can be Asp or any other amino acid
  SITE 10: note = misc_feature - Xaa can be Asp or any other amino acid
  SITE 12: note = misc_feature - Xaa can be Ser or any other amino acid
  SITE 14: note = misc_feature - Xaa can be Asn or any other amino acid
  SITE 15: note = misc_feature - Xaa can be Gln or any other amino acid
  SITE 16: note = misc_feature - Xaa can be Lys or any other amino acid
XXXIYPGXXXTXYXXXFQG

SEQ ID NO: 76 - protein, 10 aa, synthetic construct
  REGION 1..10: note = anti-CD47 antibody molecule or antigen-binding portion HCDR1
  SITE 3: note = misc_feature - Xaa can be Thr or Ser
  SITE 6: note = misc_feature - Xaa can be Asn, Ser or Asp
  SITE 8: note = misc_feature - Xaa can be Asn, Ser or Ala
  SITE 9: note = misc_feature - Xaa can be Met or Ile
  SITE 10: note = misc_feature - Xaa can be His or Gly
GYXFTXYXXX

SEQ ID NO: 77 - protein, 19 aa, synthetic construct
  REGION 1..19: note = anti-CD47 antibody molecule or antigen-binding portion HCDR2
  SITE 1: note = misc_feature - Xaa can be Met, Ile or Val
  SITE 2: note = misc_feature - Xaa can be Gly, Ala or Ser
  SITE 3: note = misc_feature - Xaa can be Thr or Ile
  SITE 8: note = misc_feature - Xaa can be Asn or Asp
  SITE 9: note = misc_feature - Xaa can be Asp or Ser
  SITE 10: note = misc_feature - Xaa can be Asp or Asn
  SITE 12: note = misc_feature - Xaa can be Ser, Lys or Arg
  SITE 14: note = misc_feature - Xaa can be Asn, Ser or His
  SITE 15: note = misc_feature - Xaa can be Gln, Pro or His
  SITE 16: note = misc_feature - Xaa can be Lys or Ser
  SITE 18: note = misc_feature - Xaa can be Lys or Gln
  SITE 19: note = misc_feature - Xaa can be Asp or Gly
XXXIYPGXXXTXYXXXFXX

SEQ ID NO: 78 - protein, 8 aa, synthetic construct
  REGION 1..8: note = anti-CD47 antibody molecule or antigen-binding portion HCDR3
  SITE 1: note = misc_feature - Xaa can be Gly or Gln
  SITE 3: note = misc_feature - Xaa can be Tyr, Ala, Cys, Asp, Glu, Phe, Gly, His, Ile, Lys, Leu, Met, Asn, Gln, Arg, Ser, Thr, Val or Trp
  SITE 5: note = misc_feature - Xaa can be Ala, Ile, Thr, Val or Trp
  SITE 6: note = misc_feature - Xaa can be Met, Ala, Glu, Lys, Leu, Pro, Gln, Arg, Ser, Thr, Val or Trp
XGXRXXDY

SEQ ID NO: 79 - protein, 10 aa, synthetic construct
  REGION 1..10: note = anti-CD47 antibody molecule or antigen-binding portion HCDR1
  SITE 3: note = misc_feature - Xaa can be Thr or Ser
  SITE 6: note = misc_feature - Xaa can be Asn or Asp
  SITE 8: note = misc_feature - Xaa can be Asn or Ala
  SITE 9: note = misc_feature - Xaa can be Met or Ile
GYXFTXYXXH

SEQ ID NO: 80 - protein, 19 aa, synthetic construct
  REGION 1..19: note = anti-CD47 antibody molecule or antigen-binding portion HCDR2
  SITE 8: note = misc_feature - Xaa can be Asn or Asp
  SITE 9: note = misc_feature - Xaa can be Asp or Ser
  SITE 10: note = misc_feature - Xaa can be Asp or Asn
  SITE 12: note = misc_feature - Xaa can be Ser, Lys or Arg
  SITE 14: note = misc_feature - Xaa can be Asn or Ser
  SITE 15: note = misc_feature - Xaa can be Gln, Pro or His
  SITE 16: note = misc_feature - Xaa can be Lys or Ser
MGTIYPGXXXTXYXXXFQG

SEQ ID NO: 81 - protein, 8 aa, synthetic construct
  REGION 1..8: note = anti-CD47 antibody molecule or antigen-binding portion HCDR3
  SITE 3: note = misc_feature - Xaa can be Tyr, Glu, Phe, Ile, Lys, Asn or Val
  SITE 6: note = misc_feature - Xaa can be Met, Glu or Gln
GGXRAXDY

SEQ ID NO: 82 - protein, 16 aa, synthetic construct
  REGION 1..16: note = anti-CD47 antibody molecule or antigen-binding portion LCDR1
  SITE 7: note = misc_feature - Xaa can be Val or Leu
  SITE 8: note = misc_feature - Xaa can be Tyr or His
  SITE 10: note = misc_feature - Xaa can be Asn, Gly, Ser or Lys
  SITE 11: note = misc_feature - Xaa can be Gly, Ala or Tyr
  SITE 12: note = misc_feature - Xaa can be Asn or Tyr
  SITE 13: note = misc_feature - Xaa can be Thr or Asn
  SITE 16: note = misc_feature - Xaa can be Gly or Asp
RSSQSLXXSXXXXYLX

SEQ ID NO: 83 - protein, 7 aa, synthetic construct
  REGION 1..7: note = anti-CD47 antibody molecule or antigen-binding portion LCDR2
  SITE 6: note = misc_feature - Xaa can be Phe or Ala
KGSNRXS

SEQ ID NO: 84 - protein, 9 aa, synthetic construct
  REGION 1..9: note = anti-CD47 antibody molecule or antigen-binding portion LCDR3
  SITE 1: note = misc_feature - Xaa can be Phe or Met
  SITE 3: note = misc_feature - Xaa can be Gly or Ala
  SITE 5: note = misc_feature - Xaa can be His or Gln
  SITE 9: note = misc_feature - Xaa can be Thr or Ile
XQXSXVPYX

SEQ ID NO: 85 - protein, 16 aa, synthetic construct
  REGION 1..16: note = anti-CD47 antibody molecule or antigen-binding portion LCDR1
  SITE 10: note = misc_feature - Xaa can be Asn or Lys
  SITE 11: note = misc_feature - Xaa can be Gly, Ala or Tyr
  SITE 12: note = misc_feature - Xaa can be Asn, Gln or Tyr
  SITE 13: note = misc_feature - Xaa can be Thr or Asn
RSSQSLLHSXXXXYLG

SEQ ID NO: 86 - protein, 7 aa, synthetic construct
  REGION 1..7: note = anti-CD47 antibody molecule or antigen-binding portion LCDR2
  SITE 2: note = misc_feature - Xaa can be Val or Gly
  SITE 6: note = misc_feature - Xaa can be Phe, Ala or Ser
KXSNRXS

SEQ ID NO: 87 - skipped

SEQ ID NO: 88 - protein, 10 aa, synthetic construct
  REGION 1..10: note = anti-CD47 antibody molecule or antigen-binding portion HCDR1
GYTFTDYNMH

SEQ ID NO: 89 - protein, 10 aa, synthetic construct
  REGION 1..10: note = anti-CD47 antibody molecule or antigen-binding portion HCDR1
GYTFTNYNIH

SEQ ID NO: 90 - protein, 19 aa, synthetic construct
  REGION 1..19: note = anti-CD47 antibody molecule or antigen-binding portion HCDR2
MGTIYPGNDDTSYSQKFQG

SEQ ID NO: 91 - protein, 19 aa, synthetic construct
  REGION 1..19: note = anti-CD47 antibody molecule or antigen-binding portion HCDR2
MGTIYPGNDDTKYSQKFQG

SEQ ID NO: 92 - protein, 19 aa, synthetic construct
  REGION 1..19: note = anti-CD47 antibody molecule or antigen-binding portion HCDR2
MGTIYPGNSDTSYNPKFQG

SEQ ID NO: 93 - protein, 8 aa, synthetic construct
  REGION 1..8: note = anti-CD47 antibody molecule or antigen-binding portion HCDR3
GGKRAMDY

SEQ ID NO: 94 - protein, 19 aa, synthetic construct
  REGION 1..19: note = anti-CD47 antibody molecule or antigen-binding portion HCDR2
MGTIYPGDSDTSYNPKFQG

SEQ ID NO: 95 - protein, 8 aa, synthetic construct
  REGION 1..8: note = anti-CD47 antibody molecule or antigen-binding portion HCDR3
GGYRAQDY

SEQ ID NO: 96 - protein, 9 aa, synthetic construct
  REGION 1..9: note = anti-CD47 antibody molecule or antigen-binding portion LCDR3
MQASHVPYT

SEQ ID NO: 97 - protein, 9 aa, synthetic construct
  REGION 1..9: note = anti-CD47 antibody molecule or antigen-binding portion LCDR3
MQGSHVPYT

SEQ ID NO: 98 - protein, 19 aa, synthetic construct
  REGION 1..19: note = anti-CD47 antibody molecule or antigen-binding portion HCDR2
MGTIYPGDSDTRYNQKFQG

SEQ ID NO: 99 - protein, 8 aa, synthetic construct
  REGION 1..8: note = anti-CD47 antibody molecule or antigen-binding portion HCDR3
GGIRAMDY

SEQ ID NO: 100 - protein, 9 aa, synthetic construct
  REGION 1..9: note = anti-CD47 antibody molecule or antigen-binding portion LCDR3
FQGSHVPYI

SEQ ID NO: 101 - protein, 19 aa, synthetic construct
  REGION 1..19: note = anti-CD47 antibody molecule or antigen-binding portion HCDR2
MGTIYPGNSDTRYNPKFQG

SEQ ID NO: 102 - protein, 9 aa, synthetic construct
  REGION 1..9: note = human germline peptide sequence with high MHC class II binding affinity in the HCDR2
FQGQVTISA

SEQ ID NO: 103 - protein, 9 aa, synthetic construct
  REGION 1..9: note = human germline peptide sequence with high MHC class II binding affinity in the HCDR2
FQGRVTITA

SEQ ID NO: 104 - protein, 10 aa, synthetic construct
  REGION 1..10: note = anti-CD47 antibody molecule or antigen-binding portion HCDR1
  SITE 6: note = misc_feature - Xaa can be Asn or a conservative substitution of Asn
  SITE 8: note = misc_feature - Xaa can be Asn or any other amino acid
  SITE 9: note = misc_feature - Xaa can be Met or a conservative substitution of Met
  SITE 10: note = misc_feature - Xaa can be His or any other amino acid
GYTFTXYXXX

SEQ ID NO: 105 - protein, 19 aa, synthetic construct
  REGION 1..19: note = anti-CD47 antibody molecule or antigen-binding portion HCDR2
  SITE 3: note = misc_feature - Xaa can be Thr or any other amino acid
  SITE 8: note = misc_feature - Xaa can be Asn or a conservative substitution of Asn
  SITE 9: note = misc_feature - Xaa can be Asp or any other amino acid
  SITE 10: note = misc_feature - Xaa can be Asp or any other amino acid
  SITE 12: note = misc_feature - Xaa can be Ser or any other amino acid
  SITE 14: note = misc_feature - Xaa can be Asn or any other amino acid
  SITE 15: note = misc_feature - Xaa can be Gln or any other amino acid
  SITE 16: note = misc_feature - Xaa can be Lys or any other amino acid
MGXIYPGXXXTXYXXXFQG

SEQ ID NO: 106 - protein, 9 aa, synthetic construct
  REGION 1..9: note = TCED+ peptide
IVYSNGNTY

SEQ ID NO: 107 - protein, 9 aa, synthetic construct
  REGION 1..9: note = HAF and TCED+ peptide
YNMHWVRQA

SEQ ID NO: 108 - protein, 9 aa, synthetic construct
  REGION 1..9: note = TCED+ peptide
YAMHWVRQA

SEQ ID NO: 109 - protein, 9 aa, synthetic construct
  REGION 1..9: note = TCED+ peptide
YNIHWVRQA

SEQ ID NO: 110 - protein, 9 aa, synthetic construct
  REGION 1..9: note = TCED+ peptide
YRAMDYWGQ

SEQ ID NO: 111 - protein, 9 aa, synthetic construct
  REGION 1..9: note = TCED+ peptide
YRAEDYWGQ

SEQ ID NO: 112 - protein, 9 aa, synthetic construct
  REGION 1..9: note = HAF peptide
LIYKGSNRF

SEQ ID NO: 113 - protein, 9 aa, synthetic construct
  REGION 1..9: note = HAF peptide
VGVYYCFQA

SEQ ID NO: 114 - protein, 10 aa, Mus musculus
GYTFTNYNMH

SEQ ID NO: 115 - protein, 5 aa, Mus musculus
NYNMH

SEQ ID NO: 116 - protein, 7 aa, Mus musculus
GYTFTNY

SEQ ID NO: 117 - protein, 8 aa, Mus musculus
GYTFTNYN

SEQ ID NO: 118 - protein, 6 aa, Mus musculus
TNYNMH

SEQ ID NO: 119 - protein, 19 aa, Mus musculus
MGTIYPGNDDTSYNQKFKD

SEQ ID NO: 120 - protein, 17 aa, Mus musculus
TIYPGNDDTSYNQKFKD

SEQ ID NO: 121 - protein, 6 aa, Mus musculus
YPGNDD

SEQ ID NO: 122 - protein, 8 aa, Mus musculus
IYPGNDDT

SEQ ID NO: 123 - protein, 16 aa, Mus musculus
IYPGNDDTSYNQKFKD

SEQ ID NO: 124 - protein, 10 aa, Mus musculus
TIYPGNDDTS

SEQ ID NO: 125 - protein, 12 aa, Mus musculus
MGTIYPGNDDTS

SEQ ID NO: 126 - protein, 8 aa, Mus musculus
GGYRAMDY

SEQ ID NO: 127 - protein, 10 aa, Mus musculus
ARGGYRAMDY

SEQ ID NO: 128 - protein, 7 aa, Mus musculus
GGYRAMD

SEQ ID NO: 129 - protein, 9 aa, Mus musculus
ARGGYRAMD

SEQ ID NO: 130 - protein, 16 aa, Mus musculus
RSSQSIVYSNGNTYLG

SEQ ID NO: 131 - protein, 11 aa, Mus musculus
QSIVYSNGNTY

SEQ ID NO: 132 - protein, 13 aa, Mus musculus
SSQSIVYSNGNTY

SEQ ID NO: 133 - protein, 12 aa, Mus musculus
VYSNGNTYLGWY

SEQ ID NO: 134 - protein, 7 aa, Mus musculus
KVSNRFS

SEQ ID NO: 135 - protein, 10 aa, Mus musculus
ALIYKVSNRF

SEQ ID NO: 136 - protein, 9 aa, Mus musculus
FQGSHVPYT

SEQ ID NO: 137 - protein, 6 aa, Mus musculus
GSHVPY

SEQ ID NO: 138 - protein, 8 aa, Mus musculus
FQGSHVPY

SEQ ID NO: 139 - protein, 117 aa, Mus musculus
QVQLQQPGAELVKPGASVMMSCKASGYTFTNYNMHWVKQTPGQGLEWIGTIYPGNDDTSYNQKFKDKATLTADKSSSAAYMQLSSLTSEDSAVYYCARGGYRAMDYWGQGTSVTVSS

SEQ ID NO: 140 - protein, 117 aa, synthetic construct
  REGION 1..117: note = h5F9G4-VH IGHV1-3
QVQLVQSGAEVKKPGASVKVSCKASGYTFTNYNMHWVRQAPGQRLEWMGTIYPGNDDTSYNQKFKDRVTITADTSASTAYMELSSLRSEDTAVYYCARGGYRAMDYWGQGTLVTVSS

SEQ ID NO: 141 - protein, 117 aa, synthetic construct
  REGION 1..117: note = VH graft IGHV5-51
EVQLVQSGAEVKKPGESLKISCKGSGYSFTNYNMHWVRQMPGKGLEWMGTIYPGNDDTSYNQKFQGQVTISADKSISTAYLQWSSLKASDTAMYYCARGGYRAMDYWGQGTLVTVSS

SEQ ID NO: 142 - protein, 112 aa, Mus musculus
DVLMTQTPLSLPVSLGDQASISCRSSQSIVYSNGNTYLGWYLQKPGQSPKLLIYKVSNRFSGVPDRFSGSGSGTDFTLKISRVEAEDLGVYHCFQGSHVPYTFGGGTKVEIK

SEQ ID NO: 143 - protein, 112 aa, synthetic construct
  REGION 1..112: note = h5F9G4-VL IGKV2-28
DIVMTQSPLSLPVTPGEPASISCRSSQSIVYSNGNTYLGWYLQKPGQSPQLLIYKVSNRFSGVPDRFSGSGSGTDFTLKISRVEAEDVGVYYCFQGSHVPYTFGQGTKLEIK

SEQ ID NO: 144 - protein, 112 aa, synthetic construct; identical to SEQ ID NO: 143
  REGION 1..112: note = IGKV2-28

SEQ ID NO: 145 - protein, 16 aa, synthetic construct
  REGION 1..16: note = anti-CD47 antibody molecule or antigen-binding portion LCDR1
RSSQSLLHSGGNTYLD

SEQ ID NO: 146 - protein, 16 aa, synthetic construct
  REGION 1..16: note = anti-CD47 antibody molecule or antigen-binding portion LCDR1
RSSQSLLHSGGNTYLG

SEQ ID NO: 147 - protein, 16 aa, synthetic construct
  REGION 1..16: note = anti-CD47 antibody molecule or antigen-binding portion LCDR1
RSSQSLLHSGYNTYLG

SEQ ID NO: 148 - protein, 16 aa, synthetic construct
  REGION 1..16: note = anti-CD47 antibody molecule or antigen-binding portion LCDR1
RSSQSLLHSNGNTYLD

SEQ ID NO: 149 - protein, 16 aa, synthetic construct
  REGION 1..16: note = anti-CD47 antibody molecule or antigen-binding portion LCDR1
RSSQSLLHSNYNTYLG

SEQ ID NO: 150 - protein, 16 aa, synthetic construct
  REGION 1..16: note = anti-CD47 antibody molecule or antigen-binding portion LCDR1
RSSQSLLQSNGNTYLG

SEQ ID NO: 151 - protein, 16 aa, synthetic construct
  REGION 1..16: note = anti-CD47 antibody molecule or antigen-binding portion LCDR1
RSSQSLLYSGGNTYLG

SEQ ID NO: 152 - protein, 16 aa, synthetic construct
  REGION 1..16: note = anti-CD47 antibody molecule or antigen-binding portion LCDR1
RSSQSLLYSNGNTYLG

SEQ ID NO: 153 - protein, 16 aa, synthetic construct
  REGION 1..16: note = anti-CD47 antibody molecule or antigen-binding portion LCDR1
RSSQSLVHSGGNTYLG

SEQ ID NO: 154 - protein, 16 aa, synthetic construct
  REGION 1..16: note = anti-CD47 antibody molecule or antigen-binding portion LCDR1
RSSQSLVHSGYNTYLG

SEQ ID NO: 155 - protein, 16 aa, synthetic construct
  REGION 1..16: note = anti-CD47 antibody molecule or antigen-binding portion LCDR1
RSSQSLVHSNGNTYLD

SEQ ID NO: 156 - protein, 16 aa, synthetic construct
  REGION 1..16: note = anti-CD47 antibody molecule or antigen-binding portion LCDR1
RSSQSLVHSNGNTYLG

SEQ ID NO: 157 - protein, 16 aa, synthetic construct
  REGION 1..16: note = anti-CD47 antibody molecule or antigen-binding portion LCDR1
RSSQSLVHSNGQTYLG

SEQ ID NO: 158 - protein, 16 aa, synthetic construct
  REGION 1..16: note = anti-CD47 antibody molecule or antigen-binding portion LCDR1
RSSQSLVHSNYNTYLG

SEQ ID NO: 159 - protein, 16 aa, synthetic construct
  REGION 1..16: note = anti-CD47 antibody molecule or antigen-binding portion LCDR1
RSSQSLVHSSGNTYLG

SEQ ID NO: 160 - protein, 16 aa, synthetic construct
  REGION 1..16: note = anti-CD47 antibody molecule or antigen-binding portion LCDR1
RSSQSLVQSNGNTYLG

SEQ ID NO: 161 - protein, 16 aa, synthetic construct
  REGION 1..16: note = anti-CD47 antibody molecule or antigen-binding portion LCDR1
RSSQSLVYSNGNTYLG

SEQ ID NO: 162 - protein, 16 aa, synthetic construct
  REGION 1..16: note = anti-CD47 antibody molecule or antigen-binding portion LCDR1
RSSQSLVYSNYNTYLG

SEQ ID NO: 163 - protein, 7 aa, synthetic construct
  REGION 1..7: note = anti-CD47 antibody molecule or antigen-binding portion LCDR2
KVSNRAS

SEQ ID NO: 164 - protein, 7 aa, synthetic construct
  REGION 1..7: note = anti-CD47 antibody molecule or antigen-binding portion LCDR2
KVSNRSS

SEQ ID NO: 165 - protein, 9 aa, synthetic construct
  REGION 1..9: note = anti-CD47 antibody molecule or antigen-binding portion LCDR3
FQALHVPYT

SEQ ID NO: 166 - protein, 9 aa, synthetic construct
  REGION 1..9: note = anti-CD47 antibody molecule or antigen-binding portion LCDR3
FQAMQTPYT

SEQ ID NO: 167 - protein, 9 aa, synthetic construct
  REGION 1..9: note = anti-CD47 antibody molecule or antigen-binding portion LCDR3
FQASHIPYT

SEQ ID NO: 168 - protein, 9 aa, synthetic construct
  REGION 1..9: note = anti-CD47 antibody molecule or antigen-binding portion LCDR3
FQASHTPYT

SEQ ID NO: 169 - protein, 9 aa, synthetic construct
  REGION 1..9: note = anti-CD47 antibody molecule or antigen-binding portion LCDR3
FQASQIPYT

SEQ ID NO: 170 - protein, 9 aa, synthetic construct
  REGION 1..9: note = anti-CD47 antibody molecule or antigen-binding portion LCDR3
FQASQTPYT

SEQ ID NO: 171 - protein, 9 aa, synthetic construct
  REGION 1..9: note = anti-CD47 antibody molecule or antigen-binding portion LCDR3
FQASRVPYT

SEQ ID NO: 172 - protein, 9 aa, synthetic construct
  REGION 1..9: note = anti-CD47 antibody molecule or antigen-binding portion LCDR3
FQATQVPYT

SEQ ID NO: 173 - protein, 9 aa, synthetic construct
  REGION 1..9: note = anti-CD47 antibody molecule or antigen-binding portion LCDR3
FQGLQVPYT

SEQ ID NO: 174 - protein, 9 aa, synthetic construct
  REGION 1..9: note = anti-CD47 antibody molecule or antigen-binding portion LCDR3
FQGSHIPYT

SEQ ID NO: 175 - protein, 9 aa, synthetic construct
  REGION 1..9: note = anti-CD47 antibody molecule or antigen-binding portion LCDR3
FQGSHTPYT

SEQ ID NO: 176 - protein, 9 aa, synthetic construct
  REGION 1..9: note = anti-CD47 antibody molecule or antigen-binding portion LCDR3
FQGSQTPYT

SEQ ID NO: 177 - protein, 9 aa, synthetic construct
  REGION 1..9: note = anti-CD47 antibody molecule or antigen-binding portion LCDR3
FQGSQVPYT

SEQ ID NO: 178 - protein, 9 aa, synthetic construct
  REGION 1..9: note = anti-CD47 antibody molecule or antigen-binding portion LCDR3
LQGSHIPYT

SEQ ID NO: 179 - protein, 9 aa, synthetic construct
  REGION 1..9: note = anti-CD47 antibody molecule or antigen-binding portion LCDR3
LQGSHTPYT

SEQ ID NO: 180 - protein, 9 aa, synthetic construct
  REGION 1..9: note = anti-CD47 antibody molecule or antigen-binding portion LCDR3
LQGSHVPYT

SEQ ID NO: 181 - protein, 9 aa, synthetic construct
  REGION 1..9: note = anti-CD47 antibody molecule or antigen-binding portion LCDR3
LQGSQTPYT

SEQ ID NO: 182 - protein, 9 aa, synthetic construct
  REGION 1..9: note = anti-CD47 antibody molecule or antigen-binding portion LCDR3
LQGSQVPYT

SEQ ID NO: 183 - protein, 9 aa, synthetic construct
  REGION 1..9: note = anti-CD47 antibody molecule or antigen-binding portion LCDR3
MQALQVPYT

SEQ ID NO: 184 - protein, 9 aa, synthetic construct
  REGION 1..9: note = anti-CD47 antibody molecule or antigen-binding portion LCDR3
MQASHIPYT

SEQ ID NO: 185 - protein, 9 aa, synthetic construct
  REGION 1..9: note = anti-CD47 antibody molecule or antigen-binding portion LCDR3
MQASHTPYT

SEQ ID NO: 186 - protein, 9 aa, synthetic construct
  REGION 1..9: note = anti-CD47 antibody molecule or antigen-binding portion LCDR3
MQASQIPYT

SEQ ID NO: 187 - protein, 9 aa, synthetic construct
  REGION 1..9: note = anti-CD47 antibody molecule or antigen-binding portion LCDR3
MQASQTPYT

SEQ ID NO: 188 - protein, 9 aa, synthetic construct
  REGION 1..9: note = anti-CD47 antibody molecule or antigen-binding portion LCDR3
MQGLHVPYT

SEQ ID NO: 189 - protein, 9 aa, synthetic construct
  REGION 1..9: note = anti-CD47 antibody molecule or antigen-binding portion LCDR3
MQGLQVPYT

SEQ ID NO: 190 - protein, 9 aa, synthetic construct
  REGION 1..9: note = anti-CD47 antibody molecule or antigen-binding portion LCDR3
MQGSHIPYT

SEQ ID NO: 191 - protein, 9 aa, synthetic construct
  REGION 1..9: note = anti-CD47 antibody molecule or antigen-binding portion LCDR3
MQGSHTPYT

SEQ ID NO: 192 - protein, 9 aa, synthetic construct
  REGION 1..9: note = anti-CD47 antibody molecule or antigen-binding portion LCDR3
MQGSQIPYT

SEQ ID NO: 193 - protein, 9 aa, synthetic construct
  REGION 1..9: note = anti-CD47 antibody molecule or antigen-binding portion LCDR3
MQGSQTPYT

SEQ ID NO: 194 - protein, 9 aa, synthetic construct
  REGION 1..9: note = anti-CD47 antibody molecule or antigen-binding portion LCDR3
MQGSQVPYT

SEQ ID NO: 195 - protein, 10 aa, synthetic construct
  REGION 1..10: note = anti-CD47 antibody molecule or antigen-binding portion HCDR1
GYSFTNYNIG

SEQ ID NO: 196 - protein, 10 aa, synthetic construct
  REGION 1..10: note = anti-CD47 antibody molecule or antigen-binding portion HCDR1
GYSFTNYNMH

SEQ ID NO: 197 - protein, 10 aa, synthetic construct
  REGION 1..10: note = anti-CD47 antibody molecule or antigen-binding portion HCDR1
GYSFTNYSIH

SEQ ID NO: 198 - protein, 10 aa, synthetic construct
  REGION 1..10: note = anti-CD47 antibody molecule or antigen-binding portion HCDR1
GYSFTSYNIH

SEQ ID NO: 199 - protein, 10 aa, synthetic construct
  REGION 1..10: note = anti-CD47 antibody molecule or antigen-binding portion HCDR1
GYSFTSYNMH

SEQ ID NO: 200 - protein, 19 aa, synthetic construct
  REGION 1..19: note = anti-CD47 antibody molecule or antigen-binding portion HCDR2
IGTIYPGDDDTRYNPKFQG

SEQ ID NO: 201 - protein, 19 aa, synthetic construct
  REGION 1..19: note = anti-CD47 antibody molecule or antigen-binding portion HCDR2
MGIIYPGDDDTRYNPKFQG

SEQ ID NO: 202 - protein, 19 aa, synthetic construct
  REGION 1..19: note = anti-CD47 antibody molecule or antigen-binding portion HCDR2
MGIIYPGDDDTRYNPSFQG

SEQ ID NO: 203 - protein, 19 aa, synthetic construct
  REGION 1..19: note = anti-CD47 antibody molecule or antigen-binding portion HCDR2
MGIIYPGDDDTSYNPKFQG

SEQ ID NO: 204 - protein, 19 aa, synthetic construct
  REGION 1..19: note = anti-CD47 antibody molecule or antigen-binding portion HCDR2
MGIIYPGDDDTSYNQKFQG

SEQ ID NO: 205 - protein, 19 aa, synthetic construct
  REGION 1..19: note = anti-CD47 antibody molecule or antigen-binding portion HCDR2
MGIIYPGDSDTRYNPKFQG

SEQ ID NO: 206 - protein, 19 aa, synthetic construct
  REGION 1..19: note = anti-CD47 antibody molecule or antigen-binding portion HCDR2
MGIIYPGDSDTRYNPSFQG

SEQ ID NO: 207 - protein, 19 aa, synthetic construct
  REGION 1..19: note = anti-CD47 antibody molecule or antigen-binding portion HCDR2
MGIIYPGDSDTRYNQKFQG

SEQ ID NO: 208 - protein, 19 aa, synthetic construct
  REGION 1..19: note = anti-CD47 antibody molecule or antigen-binding portion HCDR2
MGIIYPGDSDTSYNQSFQG

SEQ ID NO: 209 - protein, 19 aa, synthetic construct
  REGION 1..19: note = anti-CD47 antibody molecule or antigen-binding portion HCDR2
MGIIYPGNDDTRYNPKFQG

SEQ ID NO: 210 - protein, 19 aa, synthetic construct
  REGION 1..19: note = anti-CD47 antibody molecule or antigen-binding portion HCDR2
MGIIYPGNDDTRYNPSFQG

SEQ ID NO: 211 - protein, 19 aa, synthetic construct
  REGION 1..19: note = anti-CD47 antibody molecule or antigen-binding portion HCDR2
MGIIYPGNDDTRYNQKFQG

SEQ ID NO: 212 - protein, 19 aa, synthetic construct
  REGION 1..19: note = anti-CD47 antibody molecule or antigen-binding portion HCDR2
MGIIYPGNSDTRYNPKFQG

SEQ ID NO: 213 - protein, 19 aa, synthetic construct
  REGION 1..19: note = anti-CD47 antibody molecule or antigen-binding portion HCDR2
MGIIYPGNSDTRYNPSFQG

SEQ ID NO: 214 - protein, 19 aa, synthetic construct
  REGION 1..19: note = anti-CD47 antibody molecule or antigen-binding portion HCDR2
MGIIYPGNSDTRYNQKFQG

SEQ ID NO: 215 - protein, 19 aa, synthetic construct
  REGION 1..19: note = anti-CD47 antibody molecule or antigen-binding portion HCDR2
MGIIYPGNSDTRYNQRFQG

SEQ ID NO: 216 - protein, 19 aa, synthetic construct
  REGION 1..19: note = anti-CD47 antibody molecule or antigen-binding portion HCDR2
MGIIYPGNSDTRYNQSFQG

SEQ ID NO: 217 - protein, 19 aa, synthetic construct
  REGION 1..19: note = anti-CD47 antibody molecule or antigen-binding portion HCDR2
MGIIYPGNSDTRYSPKFQG

SEQ ID NO: 218 - protein, 19 aa, synthetic construct
  REGION 1..19: note = anti-CD47 antibody molecule or antigen-binding portion HCDR2
MGIIYPGNSDTRYSPSFQG

SEQ ID NO: 219 - protein, 19 aa, synthetic construct
  REGION 1..19: note = anti-CD47 antibody molecule or antigen-binding portion HCDR2
MGIIYPGNSDTSYNPKFQG

SEQ ID NO: 220 - protein, 19 aa, synthetic construct
  REGION 1..19: note = anti-CD47 antibody molecule or antigen-binding portion HCDR2
MGIIYPGNSDTSYNPRFQG

SEQ ID NO: 221 - protein, 19 aa, synthetic construct
  REGION 1..19: note = anti-CD47 antibody molecule or antigen-binding portion HCDR2
MGIIYPGNSDTSYNPSFQG

SEQ ID NO: 222 - protein, 19 aa, synthetic construct
  REGION 1..19: note = anti-CD47 antibody molecule or antigen-binding portion HCDR2
MGIIYPGNSDTSYNQKFQG

SEQ ID NO: 223 - protein, 19 aa, synthetic construct
  REGION 1..19: note = anti-CD47 antibody molecule or antigen-binding portion HCDR2
MGIIYPGNSDTSYSPKFQG

SEQ ID NO: 224 - protein, 19 aa, synthetic construct
  REGION 1..19: note = anti-CD47 antibody molecule or antigen-binding portion HCDR2
MGTIYPGDDDTRYNPKFQG

SEQ ID NO: 225 - protein, 19 aa, synthetic construct
  REGION 1..19: note = anti-CD47 antibody molecule or antigen-binding portion HCDR2
MGTIYPGDDDTRYNPSFQG

SEQ ID NO: 226 - protein, 19 aa, synthetic construct
  REGION 1..19: note = anti-CD47 antibody molecule or antigen-binding portion HCDR2
MGTIYPGDDDTRYNQKFQG

SEQ ID NO: 227 - protein, 19 aa, synthetic construct
  REGION 1..19: note = anti-CD47 antibody molecule or antigen-binding portion HCDR2
MGTIYPGDDDTRYNQNFQG

SEQ ID NO: 228 - protein, 19 aa, synthetic construct
  REGION 1..19: note = anti-CD47 antibody molecule or antigen-binding portion HCDR2
MGTIYPGDDDTRYNQSFQG

SEQ ID NO: 229 - protein, 19 aa, synthetic construct
  REGION 1..19: note = anti-CD47 antibody molecule or antigen-binding portion HCDR2
MGTIYPGDDDTRYSPKFQG

SEQ ID NO: 230 - protein, 19 aa, synthetic construct
  REGION 1..19: note = anti-CD47 antibody molecule or antigen-binding portion HCDR2
MGTIYPGDDDTRYSPSFQG

SEQ ID NO: 231 - protein, 19 aa, synthetic construct
  REGION 1..19: note = anti-CD47 antibody molecule or antigen-binding portion HCDR2
MGTIYPGDDDTRYSQKFQG

SEQ ID NO: 232 - protein, 19 aa, synthetic construct
  REGION 1..19: note = anti-CD47 antibody molecule or antigen-binding portion HCDR2
MGTIYPGDDDTSYSQKFQG

SEQ ID NO: 233 - protein, 19 aa, synthetic construct
  REGION 1..19: note = anti-CD47 antibody molecule or antigen-binding portion HCDR2
MGTIYPGDGDTSYNPSFQG

SEQ ID NO: 234 - protein, 19 aa, synthetic construct
  REGION 1..19: note = anti-CD47 antibody molecule or antigen-binding portion HCDR2
MGTIYPGDNDTSYNPSFQG

SEQ ID NO: 235 - protein, 19 aa, synthetic construct
  REGION 1..19: note = anti-CD47 antibody molecule or antigen-binding portion HCDR2
MGTIYPGDSDTKYNPKFQG

SEQ ID NO: 236 - protein, 19 aa, synthetic construct
  REGION 1..19: note = anti-CD47 antibody molecule or antigen-binding portion HCDR2
MGTIYPGDSDTRYNPSFQG

SEQ ID NO: 237 - protein, 19 aa, synthetic construct
  REGION 1..19: note = anti-CD47 antibody molecule or antigen-binding portion HCDR2
MGTIYPGDSDTRYSPKFQG

SEQ ID NO: 238 - protein, 19 aa, synthetic construct
  REGION 1..19: note = anti-CD47 antibody molecule or antigen-binding portion HCDR2
MGTIYPGDSDTRYSPRFQG

SEQ ID NO: 239 - protein, 19 aa, synthetic construct
  REGION 1..19: note = anti-CD47 antibody molecule or antigen-binding portion HCDR2
MGTIYPGDSDTRYSPSFQG

SEQ ID NO: 240 - protein, 19 aa, synthetic construct
  REGION 1..19: note = anti-CD47 antibody molecule or antigen-binding portion HCDR2
MGTIYPGDSDTRYSQKFQG

SEQ ID NO: 241 - protein, 19 aa, synthetic construct
  REGION 1..19: note = anti-CD47 antibody molecule or antigen-binding portion HCDR2
MGTIYPGDSDTRYSQSFQG

SEQ ID NO: 242 - protein, 19 aa, synthetic construct
  REGION 1..19: note = anti-CD47 antibody molecule or antigen-binding portion HCDR2
MGTIYPGDSDTSYNHKFQG

SEQ ID NO: 243 - protein, 19 aa, synthetic construct
  REGION 1..19: note = anti-CD47 antibody molecule or antigen-binding portion HCDR2
MGTIYPGDSDTSYNPKFHG

SEQ ID NO: 244 - protein, 19 aa, synthetic construct
  REGION 1..19: note = anti-CD47 antibody molecule or antigen-binding portion HCDR2
MGTIYPGDSDTSYNPRFQG

SEQ ID NO: 245 - protein, 19 aa, synthetic construct
  REGION 1..19: note = anti-CD47 antibody molecule or antigen-binding portion HCDR2
MGTIYPGDSDTSYNPSFQG

SEQ ID NO: 246 - protein, 19 aa, synthetic construct
  REGION 1..19: note = anti-CD47 antibody molecule or antigen-binding portion HCDR2
MGTIYPGDSDTSYNQKFQG

SEQ ID NO: 247 - protein, 19 aa, synthetic construct
  REGION 1..19: note = anti-CD47 antibody molecule or antigen-binding portion HCDR2
MGTIYPGDSDTSYNQSFQG

SEQ ID NO: 248 - protein, 19 aa, synthetic construct
  REGION 1..19: note = anti-CD47 antibody molecule or antigen-binding portion HCDR2
MGTIYPGDSDTSYSPKFQG

SEQ ID NO: 249 - protein, 19 aa, synthetic construct
  REGION 1..19: note = anti-CD47 antibody molecule or antigen-binding portion HCDR2
MGTIYPGDSDTSYSPSFQG

SEQ ID NO: 250 - protein, 19 aa, synthetic construct
  REGION 1..19: note = anti-CD47 antibody molecule or antigen-binding portion HCDR2
MGTIYPGDSDTSYSQKFQG

SEQ ID NO: 251 - protein, 19 aa, synthetic construct
  REGION 1..19: note = anti-CD47 antibody molecule or antigen-binding portion HCDR2
MGTIYPGNDDTKYSPKFQG

SEQ ID NO: 252 - protein, 19 aa, synthetic construct
  REGION 1..19: note = anti-CD47 antibody molecule or antigen-binding portion HCDR2
MGTIYPGNDDTRYNPKFQG

SEQ ID NO: 253 - protein, 19 aa, synthetic construct
  REGION 1..19: note = anti-CD47 antibody molecule or antigen-binding portion HCDR2
MGTIYPGNDDTRYNPSFQG

SEQ ID NO: 254 - protein, 19 aa, synthetic construct
  REGION 1..19: note = anti-CD47 antibody molecule or antigen-binding portion HCDR2
MGTIYPGNDDTRYNQKFQG

SEQ ID NO: 255 - protein, 19 aa, synthetic construct
  REGION 1..19: note = anti-CD47 antibody molecule or antigen-binding portion HCDR2
MGTIYPGNDDTRYNQSFQG

SEQ ID NO: 256 - protein, 19 aa, synthetic construct
  REGION 1..19: note = anti-CD47 antibody molecule or antigen-binding portion HCDR2
MGTIYPGNDDTRYSPKFQG

SEQ ID NO: 257 - protein, 19 aa, synthetic construct
  REGION 1..19: note = anti-CD47 antibody molecule or antigen-binding portion HCDR2
MGTIYPGNDDTRYSPSFQG

SEQ ID NO: 258 - protein, 19 aa, synthetic construct
  REGION 1..19: note = anti-CD47 antibody molecule or antigen-binding portion HCDR2
MGTIYPGNDDTRYSQKFQG

SEQ ID NO: 259 - protein, 19 aa, synthetic construct
  REGION 1..19: note = anti-CD47 antibody molecule or antigen-binding portion HCDR2
MGTIYPGDDDTSYNPKFQG

SEQ ID NO: 260 - protein, 19 aa, synthetic construct
  REGION 1..19: note = anti-CD47 antibody molecule or antigen-binding portion HCDR2
MGTIYPGDDDTSYNPSFQG

SEQ ID NO: 261 - protein, 19 aa, synthetic construct
  REGION 1..19: note = anti-CD47 antibody molecule or antigen-binding portion HCDR2
MGTIYPGDDDTSYNQKFQG

SEQ ID NO: 262 - protein, 19 aa, synthetic construct
  REGION 1..19: note = anti-CD47 antibody molecule or antigen-binding portion HCDR2
MGTIYPGNDDTSYNQSFQG

SEQ ID NO: 263 - protein, 19 aa, synthetic construct
  REGION 1..19: note = anti-CD47 antibody molecule or antigen-binding portion HCDR2
MGTIYPGNDDTSYSPKFQG

SEQ ID NO: 264 - protein, 19 aa, synthetic construct
  REGION 1..19: note = anti-CD47 antibody molecule or antigen-binding portion HCDR2
MGTIYPGNDDTSYSPRFQG

SEQ ID NO: 265 - protein, 19 aa, synthetic construct
  REGION 1..19: note = anti-CD47 antibody molecule or antigen-binding portion HCDR2
MGTIYPGNDDTSYSPSFQG

SEQ ID NO: 266 - protein, 19 aa, synthetic construct
  REGION 1..19: note = anti-CD47 antibody molecule or antigen-binding portion HCDR2
MGTIYPGNDDTSYSQSFQG

SEQ ID NO: 267 - protein, 19 aa, synthetic construct
  REGION 1..19: note = anti-CD47 antibody molecule or antigen-binding portion HCDR2
MGTIYPGNDGTRYNQKFQG

SEQ ID NO: 268 - protein, 19 aa, synthetic construct
  REGION 1..19: note = anti-CD47 antibody molecule or antigen-binding portion HCDR2
MGTIYPGNGDTSYNHKFQG

SEQ ID NO: 269 - protein, 19 aa, synthetic construct
  REGION 1..19: note = anti-CD47 antibody molecule or antigen-binding portion HCDR2
MGTIYPGNGDTSYNQKFQG

SEQ ID NO: 270 - protein, 19 aa, synthetic construct
  REGION 1..19: note = anti-CD47 antibody molecule or antigen-binding portion HCDR2
MGTIYPGNNDTSYNPKFQG

SEQ ID NO: 271 - protein, 19 aa, synthetic construct
  REGION 1..19: note = anti-CD47 antibody molecule or antigen-binding portion HCDR2
MGTIYPGNSDTRYNHSFQG

SEQ ID NO: 272 - protein, 19 aa, synthetic construct
  REGION 1..19: note = anti-CD47 antibody molecule or antigen-binding portion HCDR2
MGTIYPGNSDTRYNPSFQG

SEQ ID NO: 273 - protein, 19 aa, synthetic construct
  REGION 1..19: note = anti-CD47 antibody molecule or antigen-binding portion HCDR2
MGTIYPGNSDTRYNQKFQG

SEQ ID NO: 274 - protein, 19 aa, synthetic construct
  REGION 1..19: note = anti-CD47 antibody molecule or antigen-binding portion HCDR2
MGTIYPGNSDTRYNQSFQG

SEQ ID NO: 275 - protein, 19 aa, synthetic construct
  REGION 1..19: note = anti-CD47 antibody molecule or antigen-binding portion HCDR2
MGTIYPGNSDTRYSPKFQG

SEQ ID NO: 276 - protein, 19 aa, synthetic construct
  REGION 1..19: note = anti-CD47 antibody molecule or antigen-binding portion HCDR2
MGTIYPGNSDTRYSPSFQG

SEQ ID NO: 277 - protein, 19 aa, synthetic construct
  REGION 1..19: note = anti-CD47 antibody molecule or antigen-binding portion HCDR2
MGTIYPGNSDTRYSQKFQG

SEQ ID NO: 278 - protein, 19 aa, synthetic construct
  REGION 1..19: note = anti-CD47 antibody molecule or antigen-binding portion HCDR2
MGTIYPGNSDTSYNPRFQG

SEQ ID NO: 279 - protein, 19 aa, synthetic construct
  REGION 1..19: note = anti-CD47 antibody molecule or antigen-binding portion HCDR2
MGTIYPGNSDTSYNQKFQG

SEQ ID NO: 280 - protein, 19 aa, synthetic construct
  REGION 1..19: note = anti-CD47 antibody molecule or antigen-binding portion HCDR2
MGTIYPGNSDTSYNQSFQG

SEQ ID NO: 281 - protein, 19 aa, synthetic construct
  REGION 1..19: note = anti-CD47 antibody molecule or antigen-binding portion HCDR2
MGTIYPGNSDTSYSPKFQG

SEQ ID NO: 282 - protein, 19 aa, synthetic construct
  REGION 1..19: note = anti-CD47 antibody molecule or antigen-binding portion HCDR2
MGTIYPGNSDTSYSPSFQG

SEQ ID NO: 283 - protein, 19 aa, synthetic construct
  REGION 1..19: note = anti-CD47 antibody molecule or antigen-binding portion HCDR2
MGTIYPGNSDTSYSQKFQG

SEQ ID NO: 284 - protein, 19 aa, synthetic construct
  REGION 1..19: note = anti-CD47 antibody molecule or antigen-binding portion HCDR2
MGTIYSGNSDTSYSPSFQG

SEQ ID NO: 285 - protein, 19 aa, synthetic construct
  REGION 1..19: note = anti-CD47 antibody molecule or antigen-binding portion HCDR2
MGTIYPGNDDTRYSQSFQG

SEQ ID NO: 286 - protein, 19 aa, synthetic construct
  REGION 1..19: note = anti-CD47 antibody molecule or antigen-binding portion HCDR2
MGTIYPGNDDTSYNHKFQG

SEQ ID NO: 287 - protein, 19 aa, synthetic construct
  REGION 1..19: note = anti-CD47 antibody molecule or antigen-binding portion HCDR2
MGTIYPGNDDTSYNPKFQG

SEQ ID NO: 288 - protein, 19 aa, synthetic construct
  REGION 1..19: note = anti-CD47 antibody molecule or antigen-binding portion HCDR2
MGTIYPGNDDTSYNPSFQG

SEQ ID NO: 289 - protein, 8 aa, synthetic construct
  REGION 1..8: note = anti-CD47 antibody molecule or antigen-binding portion HCDR3
GGARAMDY

SEQ ID NO: 290 - protein, 8 aa, synthetic construct
  REGION 1..8: note = anti-CD47 antibody molecule or antigen-binding portion HCDR3
GGCRALDY

SEQ ID NO: 291 - protein, 8 aa, synthetic construct
  REGION 1..8: note = anti-CD47 antibody molecule or antigen-binding portion HCDR3
GGCRAMDY

SEQ ID NO: 292 - protein, 8 aa, synthetic construct
  REGION 1..8: note = anti-CD47 antibody molecule or antigen-binding portion HCDR3
GGCRAMGY

SEQ ID NO: 293 - protein, 8 aa, synthetic construct
  REGION 1..8: note = anti-CD47 antibody molecule or antigen-binding portion HCDR3
GGDRAMDY

SEQ ID NO: 294 - protein, 8 aa, synthetic construct
  REGION 1..8: note = anti-CD47 antibody molecule or antigen-binding portion HCDR3
GGERAMDY

SEQ ID NO: 295 - protein, 8 aa, synthetic construct
  REGION 1..8: note = anti-CD47 antibody molecule or antigen-binding portion HCDR3
GGFRAEDY

SEQ ID NO: 296 - protein, 8 aa, synthetic construct
  REGION 1..8: note = anti-CD47 antibody molecule or antigen-binding portion HCDR3
GGFRAQDY

SEQ ID NO: 297 - protein, 8 aa, synthetic construct
  REGION 1..8: note = anti-CD47 antibody molecule or antigen-binding portion HCDR3
GGGRAMDY

SEQ ID NO: 298 - protein, 8 aa, synthetic construct
  REGION 1..8: note = anti-CD47 antibody molecule or antigen-binding portion HCDR3
GGHRAEDY

SEQ ID NO: 299 - protein, 8 aa, synthetic construct
  REGION 1..8: note = anti-CD47 antibody molecule or antigen-binding portion HCDR3
GGHRAMDY

SEQ ID NO: 300 - protein, 8 aa, synthetic construct
  REGION 1..8: note = anti-CD47 antibody molecule or antigen-binding portion HCDR3
GGLRAMDY

SEQ ID NO: 301 - protein, 8 aa, synthetic construct
  REGION 1..8: note = anti-CD47 antibody molecule or antigen-binding portion HCDR3
GGLRTMDY

SEQ ID NO: 302 - protein, 8 aa, synthetic construct
  REGION 1..8: note = anti-CD47 antibody molecule or antigen-binding portion HCDR3
GGMRAMDY

SEQ ID NO: 303 - protein, 8 aa, synthetic construct
  REGION 1..8: note = anti-CD47 antibody molecule or antigen-binding portion HCDR3
GGNRAKDY

SEQ ID NO: 304 - protein, 8 aa, synthetic construct
  REGION 1..8: note = anti-CD47 antibody molecule or antigen-binding portion HCDR3
GGNRAMDY

SEQ ID NO: 305 - protein, 8 aa, synthetic construct
  REGION 1..8: note = anti-CD47 antibody molecule or antigen-binding portion HCDR3
GGQRAMDY

SEQ ID NO: 306 - protein, 8 aa, synthetic construct
  REGION 1..8: note = anti-CD47 antibody molecule or antigen-binding portion HCDR3
GGRRAMDY

SEQ ID NO: 307 - protein, 8 aa, synthetic construct
  REGION 1..8: note = anti-CD47 antibody molecule or antigen-binding portion HCDR3
GGSRAKDY

SEQ ID NO: 308 - protein, 8 aa, synthetic construct
  REGION 1..8: note = anti-CD47 antibody molecule or antigen-binding portion HCDR3
GGSRAMDY

SEQ ID NO: 309 - protein, 8 aa, synthetic construct
  REGION 1..8: note = anti-CD47 antibody molecule or antigen-binding portion HCDR3
GGTRAMDY

SEQ ID NO: 310 - protein, 8 aa, synthetic construct
  REGION 1..8: note = anti-CD47 antibody molecule or antigen-binding portion HCDR3
GGVRATDY

SEQ ID NO: 311 - protein, 8 aa, synthetic construct
  REGION 1..8: note = anti-CD47 antibody molecule or antigen-binding portion HCDR3
GGWRAMDY

SEQ ID NO: 312 - protein, 8 aa, synthetic construct
  REGION 1..8: note = anti-CD47 antibody molecule or antigen-binding portion HCDR3
GGYHAMDY

SEQ ID NO: 313 - protein, 8 aa, synthetic construct
  REGION 1..8: note = anti-CD47 antibody molecule or antigen-binding portion HCDR3
GGYKAMDY

SEQ ID NO: 314 - protein, 8 aa, synthetic construct
  REGION 1..8: note = anti-CD47 antibody molecule or antigen-binding portion HCDR3
GGYRAADY

SEQ ID NO: 315 - protein, 8 aa, synthetic construct
  REGION 1..8: note = anti-CD47 antibody molecule or antigen-binding portion HCDR3
GGYRALDY

SEQ ID NO: 316 - protein, 8 aa, synthetic construct
  REGION 1..8: note = anti-CD47 antibody molecule or antigen-binding portion HCDR3
GGYRAPDY

SEQ ID NO: 317 - protein, 8 aa, synthetic construct
  REGION 1..8: note = anti-CD47 antibody molecule or antigen-binding portion HCDR3
GGYRARDY

SEQ ID NO: 318 - protein, 8 aa, synthetic construct
  REGION 1..8: note = anti-CD47 antibody molecule or antigen-binding portion HCDR3
GGYRASDY

SEQ ID NO: 319 - protein, 8 aa, synthetic construct
  REGION 1..8: note = anti-CD47 antibody molecule or antigen-binding portion HCDR3
GGYRATDY

SEQ ID NO: 320 - protein, 8 aa, synthetic construct
  REGION 1..8: note = anti-CD47 antibody molecule or antigen-binding portion HCDR3
GGYRAVDY

SEQ ID NO: 321 - protein, 8 aa, synthetic construct
  REGION 1..8: note = anti-CD47 antibody molecule or antigen-binding portion HCDR3
GGYRIMDY

SEQ ID NO: 322 - protein, 8 aa, synthetic construct
  REGION 1..8: note = anti-CD47 antibody molecule or antigen-binding portion HCDR3
GGYRTEDY

SEQ ID NO: 323 - protein, 8 aa, synthetic construct
  REGION 1..8: note = anti-CD47 antibody molecule or antigen-binding portion HCDR3
GGYRTMDY

SEQ ID NO: 324 - protein, 8 aa, synthetic construct
  REGION 1..8: note = anti-CD47 antibody molecule or antigen-binding portion HCDR3
GGYRTTDY

SEQ ID NO: 325 - protein, 8 aa, synthetic construct
  REGION 1..8: note = anti-CD47 antibody molecule or antigen-binding portion HCDR3
GGYRVMDY

SEQ ID NO: 326 - protein, 8 aa, synthetic construct
  REGION 1..8: note = anti-CD47 antibody molecule or antigen-binding portion HCDR3
GGYRWMDY

SEQ ID NO: 327 - protein, 8 aa, synthetic construct
  REGION 1..8: note = anti-CD47 antibody molecule or antigen-binding portion HCDR3
QGYRAMDY

SEQ ID NO: 328 - protein, 16 aa, synthetic construct
  REGION 1..16: note = anti-CD47 antibody molecule or antigen-binding portion LCDR1
RSSQSLLHSKGNTYLG

SEQ ID NO: 329 - protein, 16 aa, synthetic construct
  REGION 1..16: note = anti-CD47 antibody molecule or antigen-binding portion LCDR1
RSSQSLLHSNYNTYLG

SEQ ID NO: 330 - protein, 7 aa, synthetic construct
  REGION 1..7: note = anti-CD47 antibody molecule or antigen-binding portion LCDR2
KGSNRAS

SEQ ID NO: 331 - protein, 19 aa, synthetic construct
  REGION 1..19: note = anti-CD47 antibody molecule or antigen-binding portion HCDR2
MGTIYPGNSDTSYNHKFQG

SEQ ID NO: 332 - protein, 19 aa, synthetic construct
  REGION 1..19: note = anti-CD47 antibody molecule or antigen-binding portion HCDR2
MGTIYPGNDNTKYNQKFQG

SEQ ID NO: 333 - protein, 8 aa, synthetic construct
  REGION 1..8: note = anti-CD47 antibody molecule or antigen-binding portion HCDR3
GGNRAEDY

(19) United States
(12) Patent Application Publication
(10) Pub. No.: US 2026/0015420 A1
(43) Pub. Date: Jan. 15, 2026

(54) CD47 BINDING AGENTS

(71) Applicant: CENTESSA PHARMACEUTICALS (UK) LIMITED, Altrincham (GB)

(72) Inventor: William James Jonathan FINLAY, Glasgow (GB)

(21) Appl. No.: 19/192,994

(22) Filed: Apr. 29, 2025

Related U.S. Application Data
(63) Continuation of application No. 17/041,100, filed on Sep. 24, 2020, now Pat. No. 12,421,310.

(30) Foreign Application Priority Data
Mar. 27, 2018 (GB) 1804860.3
Aug. 22, 2018 (GB) 1813693.7

(51) Int. Cl. C07K 16/28; A61K 39/00; A61K 45/06; A61K 47/68

(52) U.S. Cl. CPC C07K 16/2803; A61K 45/06; A61K 47/6849; A61K 2039/505; C07K 2317/14; C07K 2317/24; C07K 2317/31; C07K 2317/565; C07K 2317/567; C07K 2317/622

(57) ABSTRACT

Provided herein are antibody molecules that bind specifically to CD47 and related nucleic acid molecules, vectors and host cells. Also provided herein are medical uses of such antibody molecules.

### CROSS-REFERENCE TO RELATED APPLICATIONS

This application is a Continuation of U.S. application Ser. No. 17/041,100, filed Sep. 24, 2020, which is a 35 U.S.C. § 371 national phase application of International Patent Application No. PCT/EP2019/057723, filed on Mar. 27, 2019, which claims the benefit of GB Patent Application No. 1813693.7, filed on Aug. 22, 2018 and GB Patent Application No. 1804860.3, filed on Mar. 27, 2018, the disclosure of which are hereby incorporated by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: 60509-228.301_SL.xml, created on Apr. 28, 2025, file size 5,591,488 bytes).

### FIELD OF THE INVENTION

The invention relates to antibody molecules binding specifically to CD47 (Cluster of Differentiation 47, also known as integrin associated protein [IAP]) and medical uses thereof.

### BACKGROUND OF THE INVENTION

CD47 (also known as integrin associated protein [IAP]) is a transmembrane protein that belongs to the immunoglobulin superfamily and binds to several known partners, including: membrane integrins, thrombospondin-1 (TSP-1) and signal-regulatory protein alpha (SIRPα). CD47 is associated with a range of cellular processes, including apoptosis, proliferation, adhesion, and migration of cells and, importantly, it plays a key role in immune and angiogenic responses. CD47-SIRPα signalling is a critical molecular interaction that inhibits the activation of phagocytosis by macrophages and other myeloid cells. This promotes the survival of tumour cells and therefore acts as a myeloid lineage-specific immune checkpoint.

Preclinical evidence suggests that blocking CD47-SIRPα signalling can enhance the phagocytic activity of macrophages and inhibit the growth of xenografts in numerous experimental models of both haematological and solid malignancies. As macrophage activity is also a recognised factor in the biology of inflammation-associated tissue remodelling such as tissue fibrosis and the formation of atherosclerotic plaques, the CD47-SIRPα signalling axis is also of considerable therapeutic potential in non-cancerous diseases. Hence, anti-CD47 mAbs have the potential to act as immunotherapeutic agents in cancer and other settings, and to amplify the effectiveness of currently established therapies.

The majority of currently approved antibody therapeutics are derived from immunized rodents. Many of those antibodies have undergone a process known as “humanization”, via the “grafting” of murine CDRs into human v-gene framework sequences (see Nelson et al., 2010, Nat Rev Drug Discov 9:767-774). This process is often inaccurate and leads to a reduction in target binding affinity of the resulting antibody. To return the binding affinity of the original antibody, murine residues are usually introduced at key positions in the variable domain frameworks of the grafted v-domains (also known as “back-mutations”).

While antibodies humanized via CDR grafting and back mutations have been shown to induce lower immune response rates in the clinic in comparison to those with fully murine v-domains, antibodies humanized using this basic grafting method still carry significant clinical development risks due to the potential physical instability and immunogenicity motifs still housed in the grafted CDR loops. Antibodies such as anti-CD47, which potentially engage immune effector functions as part of their mechanism of action, are at particularly high risk for immunogenicity as they can encourage phagocytosis of CD47+target cells, leading to antigen processing of the antibody along with the target cell. These anti-drug antibody responses in the patient can reduce drug half-life, potency and safety during clinical use. As animal testing of protein immunogenicity is often non-predictive of immune responses in man, antibody engineering for therapeutic use focuses on minimizing predicted human T-cell epitope content, non-human germline amino acid content and aggregation potential in the purified protein.

The ideal humanized agonistic anti-CD47 antibody would therefore have as many identical residues as possible in the v-domains to those found in both the frameworks and CDRs of well-characterized human germline sequences. Townsend et al. (2015; PNAS 112:15354-15359) describe a method for generating antibodies in which CDRs derived from rat, rabbit and mouse antibodies were grafted into preferred human frameworks and then subject to a 30) human germ-lining approach termed “Augmented Binary Substitution”. Although the approach demonstrated a fundamental plasticity in the original antibody paratopes, in the absence of highly accurate antibody-antigen co-crystal structural data, it is still not possible to reliably predict which individual residues in the CDR loops of any given antibody can be converted to human germline, and in what combination. Additionally, the Townsend et al. study did not address the addition of mutagenesis beyond the residues found in the human germline at positions where the removal of development risk motifs might be beneficial. This is a technological limitation which renders the process inherently inefficient, leaving antibodies at high risk of development difficulties. In addition, it cannot currently be accurately predicted what modifications in distal positions of the protein sequence of an individual v-domain, or even on the partner v-domain, might facilitate the removal of risk motifs while maintaining antigen binding affinity and specificity.

CDR germ-lining and development quality optimisation is thus a complex, multifactorial problem, as multiple functional properties of the molecule should preferably be maintained, including in this instance: target binding specificity, affinity to CD47 from both human and animal test species (e.g. cynomolgus monkey, also known as the crab-eating macaque, i.e. Macaca fascicularis), v-domain biophysical stability and/or IgG expression yield. Antibody engineering studies have shown that mutation of even single residue positions in key CDRs can have dramatic effects on all of these desired molecular properties.

WO2011143624A2 describes an antagonistic murine anti-CD47 IgG molecule termed “5F9G4”, and also the preparation of humanized forms of 5F9G4 (h5F9G4). Those humanized forms of 5F9G4 were produced using classical humanization techniques, i.e. by grafting of Kabat-defined murine CDRs into human heavy and light chain framework sequences, with some of the human framework residues being potentially back-mutated to the correspondingly positioned 5F9G4 murine residues. For reasons noted above, such humanized forms of 5F9G4 described in WO2011143624A2 are not ideal.

### SUMMARY OF THE INVENTION

The present invention provides a number of anti-CD47 antibodies and medical uses thereof.

According to one aspect of the invention, there is provided an antibody molecule or an antigen-binding portion thereof, which specifically binds to human CD47, and optionally also to cynomolgus monkey CD47, wherein the antibody comprises a heavy chain variable (VH) region and a light chain variable (VL) region. In some embodiments, the VH region comprises an IGHV5-51-based or an IGHV1-3-based framework. In some embodiments, the VL region comprises an IGKV2-28-based framework.

In the IGHV5-51 background, the anti-CD47 antibody molecule or antigen-binding portion may comprise a heavy chain variable region with:
an HCDR1 having amino acids in sequence in the following order: G-Y-S-F-T-N or a conservative substitution of N (such as S)-Y-N or a conservative substitution of N (such as S)-M or a conservative substitution of M (such as I)-H or any amino acid (such as G) (SEQ ID NO: 28);
an HCDR2 having amino acids in sequence in the following order: M or a conservative substitution of M (for example, I)-G-T or any amino acid (such as I)—I—Y-P-G-N or any amino acid (such as D)-D or any amino acid (such as S)-D-T-S or any amino acid (such as R)—Y—N or any amino acid (such as S, H)-Q or any amino acid (such as P)-K or any amino acid (such as S)-F-Q/K-G/D (SEQ ID NO:29); and
an HCDR3 having amino acids in sequence in the following order: G or any amino acid (such as Q)-G-Y or any amino acid (such as A, C, D, E, F, G, H, I, K, L, M, N, Q, R, S, T, V, W)-R-A or any amino acid (such as I, T, V, W)-M or any amino acid (such as A, E, K, L, P, Q, R, S, T, V, W)-D-Y (SEQ ID NO:30).

In the IGHV1-3 background, the anti-CD47 antibody molecule or antigen-binding portion may comprise a heavy chain variable region with:
an HCDR1 having amino acids in sequence in the following order: G-Y-T-F-T-N or any amino acid (such as D)-Y-N or any amino acid (such as A)-M-H (SEQ ID NO:31);
an HCDR2 having amino acids in sequence in the following order: M-G-T-I-Y-P-G-N-D-D or any amino acid (such as N)-T-S or any amino acid (such as K)-Y-N or any amino acid (such as S)-Q-K-F-Q-G (SEQ ID NO:32); and
an HCDR3 having amino acids in sequence in the following order: G or any amino acid (such as Q)-G-Y or any amino acid (such as A, C, D, E, F, G, H, I, K, L, M, N, Q, R, S, T, V, W)-R-A or any amino acid (such as I, T, V, W)-M or any amino acid (such as A, E, K, L, P, Q, R, S, T, V, W)-D-Y (SEQ ID NO:30).

In aspects of the invention, the HCDR1 of the antibody molecule or antigen-binding portion may exclude the sequence GYTFTNYNMH (SEQ ID NO:33; 5F9G4 murine/humanized antibody HCDR1 disclosed in WO2011143624A2; US20130142786A1), the HCDR2 of the antibody molecule or antigen-binding portion may exclude the sequence MGTIYPGNDDTSYNQKFKD (SEQ ID NO:34; 5F9G4 murine/humanized antibody HCDR2 disclosed in WO2011143624A2; US20130142786A1), and/or the HCDR3 of the antibody molecule or antigen-binding portion may exclude the sequence GGYRAMDY (SEQ ID NO: 35; 5F9G4 murine/humanized antibody HCDR3 disclosed in WO2011143624A2; US20130142786A1).

The antibody molecule or antigen-binding portion may further comprise a light chain variable region with:
an LCDR1 having amino acids in sequence in the following order: R-S-S-Q-S-I or a conservative substitution of I (such as L)-V or a conservative substitution of V (such as L)-Y or a conservative substitution of Y (such as H)-S-N or any amino acid (such as G, S, K)-G or any amino acid (such as A, Y)-N or any amino acid (such as Q, Y)-T or any amino acid (such as N)-Y-L-G or any amino acid (such as D) (SEQ ID NO:36);
an LCDR2 having amino acids in sequence in the following order: K-V or any amino acid (such as G)-S-N-R-F or any amino acid (such as A, S)-S(SEQ ID NO:37); and an LCDR3 having amino acids in sequence in the following order: F or any amino acid (such as L, M)-Q-G or any amino acid (such as A)-S or any amino acid (such as T, L, M)-H or any amino acid (such as Q, R)-V or any amino acid (such as I, T)-P-Y-T or any amino acid (such as I) (SEQ ID NO:38).

In aspects of the invention, the LCDR1 of the antibody molecule or antigen-binding portion may exclude the sequence RSSQSIVYSNGNTYLG (SEQ ID NO:39; 5F9G4 murine/humanized antibody LCDR1 disclosed in WO2011143624A2; US20130142786A1), and/or the LCDR2 of the antibody molecule or antigen-binding portion may exclude the sequence KVSNRFS (SEQ ID NO:40; 5F9G4 murine/humanized antibody LCDR2 disclosed in WO2011143624A2; US20130142786A1), and/or the LCDR3 of the antibody molecule or antigen binding portion may exclude the sequence FQGSHVPYT (SEQ ID NO:41).

In some aspects, disclosed herein is an anti-CD47 antibody or an antigen-binding portion thereof, wherein the antibody comprises a heavy chain variable (VH) region and a light chain variable (VL) region, wherein
(a) the HCDR1 comprises the amino acid sequence G-Y-X₁-F-T-X₂-Y-X₃-X₄-X₅, wherein X₁ is S or a conservative substitution of S, X₂ is N or a conservative substitution of N, X₃ is N or any other amino acid, X₄ is M or a conservative substitution of M and X₅ is H or any other amino acid (SEQ ID NO:42);
(b) the HCDR2 comprises the amino acid sequence X₁-X₂-X₃-I-Y-P-G-X₄-X₅-X₆-T-X₇-Y-X₈-X₉-X₁₀-F-Q-G, wherein X₁ is M or a conservative substitution of M, X₂ is G or a conservative substitution of G, X₃ is T or any other amino acid, X₄ is N or a conservative substitution of N, X₅ is D or any other amino acid, X₆ is D or any other amino acid, X₇ is S or any other amino acid, X₈ is N or any other amino acid, X₉ is Q or any other amino acid and X₁₀ is K or any other amino acid (SEQ ID NO:43);
(c) the HCDR3 comprises the amino acid sequence X₁-G-X₂-R-X₃-X₄-D-Y, wherein X₁ is G or any other amino acid, X₂ is Y or any other amino acid, X₃ is A or any other amino acid and X₄ is M or any other amino acid (SEQ ID NO:44);
(d) the LCDR1 comprises the amino acid sequence R—S-S-Q-S-X₁-X₂-X₃-S-X₄-X₅-X₆-X₇-Y-L-X₈, wherein X₁ is I or a conservative substitution of I, X₂ is V or a conservative substitution of V, X₃ is Y or a conservative substitution of Y, X₄ is N or any other amino acid, X₅ is G or any other amino acid, X₆ is N or any other amino acid, X₇ is T or any other amino acid and X₈ is G or any other amino acid (SEQ ID NO:45);
(e) the LCDR2 comprises the amino acid sequence K-X₁—S—N-R-X₂-S, wherein X₁ is V or any other amino acid and X₂ is F or any other amino acid (SEQ ID NO:46); and
(f) the LCDR3 comprises the amino acid sequence X₁-Q-X₂-X₃-X₄-X₅-P-Y-X₆, wherein X₁ is F or any other amino acid, X₂ is G or any other amino acid, X₃ is S or any other amino acid, X₄ is H or any other amino acid, X₅ is V or any other amino acid, X₆ is T or any other amino acid.

In some aspects, the invention provides an anti-CD47 antibody or an antigen-binding portion thereof, wherein the antibody comprises a heavy chain variable (VH) region and a light chain variable (VL) region, wherein
(a) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNANTYLG (SEQ ID NO:55), LCDR2 of KGSNRFS (SEQ ID NO:52) and LCDR3 of FQASHVPYT (SEQ ID NO:53);
(b) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNGNTYLG (SEQ ID NO:51), LCDR2 of KGSNRFS (SEQ ID NO:52) and LCDR3 of FQASHVPYT (SEQ ID NO:53);
(c) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNAYNYLG (SEQ ID NO:56), LCDR2 of KGSNRFS (SEQ ID NO:52) and LCDR3 of FQASHVPYT (SEQ ID NO:53);
(d) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNAYNYLG (SEQ ID NO:56), LCDR2 of KGSNRFS (SEQ ID NO:52) and LCDR3 of FQASQVPYT (SEQ ID NO:54);
(e) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNANTYLG (SEQ ID NO:55), LCDR2 of KGSNRFS (SEQ ID NO:52) and LCDR3 of FQASQVPYT (SEQ ID NO:54);
(f) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNGNTYLG (SEQ ID NO:51), LCDR2 of KGSNRFS (SEQ ID NO:52) and LCDR3 of FQASQVPYT (SEQ ID NO:54);
(g) the VH region amino acid sequence comprises HCDR1 of GYSFTNYNIH (SEQ ID NO: 57), HCDR2 of MGTIYPGNSDTSYNPSFQG (SEQ ID NO: 61) and HCDR3 of GGVRAMDY (SEQ ID NO:62); and the VL region amino acid sequence comprises LCDR1 of RSSQSLLHSNGNTYLG (SEQ ID NO:51), LCDR2 of KGSNRFS (SEQ ID NO:52) and LCDR3 of MQASQVPYT (SEQ ID NO:63); or
(h) the VH region amino acid sequence comprises HCDR1 of GYSFTNYNIH (SEQ ID NO: 57), HCDR2 of MGTIYPGDSDTRYNPKFQG (SEQ ID NO:58) and HCDR3 of GGYRAEDY (SEQ ID NO:59); and the VL region amino acid sequence comprises LCDR1 of RSSQSLLHSNGNTYLG (SEQ ID NO:51), LCDR2 of KGSNRFS (SEQ ID NO:52) and LCDR3 of MQGSHVPY (SEQ ID NO:60).

In some aspects, disclosed herein is an anti-CD47 antibody or an antigen-binding portion thereof, wherein the antibody comprises a heavy chain variable (VH) region and a light chain variable (VL) region, wherein
the VH region amino acid sequence comprises:
(a) HCDR1 of SEQ ID NO: 48 or SEQ ID NO: 57;
(b) HCDR2 of SEQ ID NO: 49 or SEQ ID NO: 61; and
(c) HCDR3 of SEQ ID NO: 50 or SEQ ID NO: 62; and
the VL region amino acid sequence comprises:
(a′) LCDR1 of SEQ ID NO: 51, SEQ ID NO: 55 or SEQ ID NO: 56;
(b′) LCDR2 of SEQ ID NO: 52; and
(c′) LCDR3 of SEQ ID NO: 53, SEQ ID NO: 54 or SEQ ID NO: 63.

In some aspects, disclosed herein is an anti-CD47 antibody or an antigen-binding portion thereof, wherein the antibody comprises a heavy chain variable (VH) region and a light chain variable (VL) region, wherein
(a) the VH region amino acid sequence comprises SEQ ID NO:1 and the VL region amino acid sequence comprises SEQ ID NO:2;
(b) the VH region amino acid sequence comprises SEQ ID NO:3 and the VL region amino acid sequence comprises SEQ ID NO:4;
(c) the VH region amino acid sequence comprises SEQ ID NO:5 and the VL region amino acid sequence comprises SEQ ID NO:6;
(d) the VH region amino acid sequence comprises SEQ ID NO:7 and the VL region amino acid sequence comprises SEQ ID NO:8;
(e) the VH region amino acid sequence comprises SEQ ID NO:9 and the VL region amino acid sequence comprises SEQ ID NO:10;
(f) the VH region amino acid sequence comprises SEQ ID NO:11 and the VL region amino acid sequence comprises SEQ ID NO:12;
(g) the VH region amino acid sequence comprises SEQ ID NO:13 and the VL region amino acid sequence comprises SEQ ID NO:14; or
(h) the VH region amino acid sequence comprises SEQ ID NO:15 and the VL region amino acid sequence comprises SEQ ID NO:16.

Also provided according to the invention is an immunoconjugate comprising the antibody molecule or antigen-binding portion thereof as defined herein linked to a therapeutic agent.

In another aspect the invention provides a nucleic acid molecule encoding the antibody molecule or antigen-binding portion thereof as defined herein.

Further provided is a vector comprising the nucleic acid molecule of the invention.

Also provided is a host cell comprising the nucleic acid molecule or the vector of the invention as defined herein.

In a further aspect there is provided a method of producing an anti-CD47 antibody and/or an antigen-binding portion thereof, comprising culturing the host cell of the invention under conditions that result in expression and/or production of the antibody and/or the antigen-binding portion thereof, and isolating the antibody and/or the antigen-binding portion thereof from the host cell or culture.

In another aspect of the invention there is provided a pharmaceutical composition comprising the antibody molecule or antigen-binding portion thereof of the invention as defined herein, or the nucleic acid molecule of the invention as defined herein, or the vector of the invention as defined herein.

Further provided is a method for enhancing an immune response in a subject, comprising administering an effective amount of the antibody molecule or antigen-binding portion thereof of the invention as defined herein, or the immunoconjugate of the invention as defined herein, or the nucleic acid molecule of the invention as defined herein, or the vector of the invention as defined herein, or the pharmaceutical composition of the invention as defined herein.

In a further aspect there is provided a method for treating or preventing cancer in a subject, comprising administering an effective amount of the antibody molecule or antigen-binding portion thereof of the invention as defined herein, or the immunoconjugate of the invention as defined herein, or the nucleic acid molecule of the invention as defined herein, or the vector of the invention as defined herein, or the pharmaceutical composition of the invention as defined herein.

Further provided herein is an antibody molecule or antigen-binding portion thereof as defined herein, or the scFv as defined herein, or the immunoconjugate as defined herein, or the nucleic acid molecule as defined herein, or the vector as defined herein, or the pharmaceutical composition as defined herein, for use as a medicament. The invention also provides an antibody molecule or antigen-binding portion thereof of the invention as defined herein, or the immunoconjugate of the invention as defined herein, or the nucleic acid molecule of the invention as defined herein, or the vector of the invention as defined herein, or the pharmaceutical composition of the invention as defined herein, for use in the treatment of cancer.

In another aspect the invention provides the antibody molecule, or antigen-binding portion thereof, or the immunoconjugate, or the nucleic acid molecule, or the vector for use, or the method of treatment of the invention as defined herein, for separate, sequential or simultaneous use in a combination with a second therapeutic agent, for example an anti-cancer agent.

In a further aspect there is provided the use of an antibody molecule or antigen-binding portion thereof of the invention as defined herein, or an immunoconjugate of the invention as defined herein, or a nucleic acid molecule of the invention as defined herein, or a vector of the invention as defined herein, or a pharmaceutical composition of the invention as defined herein, in the manufacture of a medicament for the treatment of cancer.

The invention also provides a method for treating or preventing an autoimmune disease or an inflammatory disease in a subject, comprising administering an effective amount of the antibody molecule or antigen-binding portion thereof as defined herein, or the immunoconjugate as defined here, or the nucleic acid molecule as defined herein, or the vector as defined herein, or the pharmaceutical composition as defined herein.

The autoimmune disease or inflammatory disease may be selected in all aspects from the group consisting of: arthritis, asthma, multiple sclerosis, psoriasis, Crohn's disease, inflammatory bowel disease, lupus, Grave's disease and Hashimoto's thyroiditis, and ankylosing spondylitis.

Also provided is an antibody molecule or antigen-binding portion thereof as defined herein, or the immunoconjugate as defined herein, or the nucleic acid molecule as defined herein, or the vector as defined herein, or the pharmaceutical composition as defined herein, for use in the treatment of an autoimmune disease or an inflammatory disease.

Further provided is the use of an antibody molecule or antigen-binding portion thereof as defined herein, or an immunoconjugate as defined herein, or a nucleic acid molecule as defined herein, or a vector as defined herein, or a pharmaceutical composition as defined herein, in the manufacture of a medicament for the treatment of an autoimmune disease or an inflammatory disease.

The invention also provides a method for treating or preventing a cardiovascular disease or a fibrotic disease in a subject, comprising administering an effective amount of the antibody molecule or antigen-binding portion thereof as defined herein, or the immunoconjugate as defined here, or the nucleic acid molecule as defined herein, or the vector as defined herein, or the pharmaceutical composition as defined herein.

Also provided is an antibody molecule or antigen-binding portion thereof as defined herein, or the immunoconjugate as defined herein, or the nucleic acid molecule as defined herein, or the vector as defined herein, or the pharmaceutical composition as defined herein, for use in the treatment of a cardiovascular disease or a fibrotic disease.

Further provided is the use of an antibody molecule or antigen-binding portion thereof as defined herein, or an immunoconjugate as defined herein, or a nucleic acid molecule as defined herein, or a vector as defined herein, or a pharmaceutical composition as defined herein, in the manufacture of a medicament for the treatment of an autoimmune disease, an inflammatory disease or a fibrotic disease.

The cardiovascular disease in any aspect of the invention may for example be coronary heart disease or atherosclerosis.

The fibrotic disease in any aspect of the invention may for example be myocardial infarction, angina, osteoarthritis, pulmonary fibrosis, cystic fibrosis, bronchitis or asthma.

The invention also provides a method of producing an antibody molecule which specifically binds to human CD47 and optionally also to cynomolgus monkey CD47, or an antigen-binding portion thereof, comprising the steps of:
(1) grafting anti-CD47 CDRs from a non-human source into a human v-domain framework to produce a humanized anti-CD47 antibody molecule or antigen-binding portion thereof;
(2) generating a phage library of clones of the humanized anti-CD47 antibody molecule or antigen-binding portion thereof comprising one or more mutations in the CDRs;
(3) screening the phage library for binding to human CD47 and optionally also to cynomolgus monkey CD47;
(4) selecting clones from the screening step (3) having binding specificity to human CD47 and optionally also to cynomolgus monkey CD47; and
(5) producing an antibody molecule which specifically binds to human CD47 and optionally also to cynomolgus monkey CD47, or an antigen-binding portion thereof from clones selected from step (4).

The method may comprise a further step of producing additional clones based on the clones selected in step (4), for example based on further exploratory mutagenesis at specific positions in the CDRs of the clones selected in step (4), to enhance humanization and/or minimise human T cell epitope content and/or improve manufacturing properties in the antibody molecule or antigen-binding portion thereof produced in step (5).

### BRIEF DESCRIPTION OF THE FIGURES

FIG. 1A-FIG. 1F. Assay technology known under the trademark HTRF®-based competition screening of library-derived anti-CD47 scFvs against h5F9G4 IgG1 and hSIRPα-Fc binding to human and cyno CD47-Fc proteins. Clones were derived from separate phage selection branches on biotinylated human and/or cynomolgus monkey CD47-Fc proteins in each round. After multiple rounds of selection in two branches, library-derived clones (black circles) were screened for binding against both human and cyno CD47-Fc and epitope blocking (measured via signal reduction) of h5F9G4 IgG1 and hSIRPxx-Fc. Round 2 outputs (FIG. 1A, FIG. 1B). Round 3 outputs (FIG. 1C, FIG. 1D). Round 4 outputs (FIG. 1E, FIG. 1F). Negative control (non-CD47-binding) and positive control h5F9G4 scFvs are represented in grey crossed circle and black circle with grey border, respectively. In addition, an IGHV5-51/IGKV2-28 graft scFv was also included on each plate (grey hexagon).

FIG. 2A-FIG. 2B. Analysis of CDR residue tolerance for mutation to germline. A plot of murine amino acid retention frequencies in the CDRs of the ELISA-positive population of 269 unique scFv clones is shown for VH (SEQ ID NOs: 64-66) (FIG. 2A) and VL (SEQ ID NOS: 67-69) (FIG. 2B) domains, respectively. Only those residues targeted for human/murine residue mutagenesis are plotted, other than in the HCDR3. CDR residues noted in parentheses on the X-axes were identical to those found in the human germlines used for grafting (IGKV2-28 and IGHV5-51). Those residues in the HCDR2 that are not in parentheses, but whose values are set at 0, were mutated to human germline during the grafting process. In both plots the dashed line in grey at 75% represents the cut off for tolerance of murine residue replacement by human germline.

FIG. 3A-FIG. 3B. Direct titration ELISA for library-derived lead IgGs binding to human and cyno CD47-Fc proteins. Chimeric anti-CD47 (m5F9G4) and library-derived clones in human IgG1null format were titrated (in μg/ml) in a direct binding ELISA against human (FIG. 3A) and cyno (FIG. 3B) CD47-Fc proteins. The m5F9G4 and library-derived clones demonstrated binding activity against both orthologs of CD47.

FIG. 4A-FIG. 4B. ELISA-based CD47-Fc-SIRPα competition assay for library-derived leads. ELISA binding signal for human (FIG. 4A) and cyno (FIG. 4B) CD47-Fc proteins to plate-bound human SIRPαwas examined in the presence of titrated competitor IgGs in IgG1null format, D-A6 scFv-Fc, plus Isotype IgG1, IGHV5-51 graft, h5F9G4 and m5F9G4 in IgG1null format.

FIG. 5A-FIG. 5B. Assay technology known under the trademark HTRF®-based solution-phase, high-sensitivity, CD47 epitope competition assay for library-derived leads. Assay technology known under the trademark HTRF® binding signal for the h5F9G4 IgG to human or cyno CD47 was examined in the presence of titrated competitor IgGs including library-derived leads, plus Isotype IgG1 and unlabelled h5F9G4 and m5F9G4. All library-derived leads exhibited concentration-dependent inhibition of h5F9G4 binding to human CD47, suggesting maintenance of a shared epitope, but lower binding affinity (FIG. 30) 5A). Both D-A6 proteins (IgG and scFv-Fc) demonstrated the ability to fully inhibit h5F9G4 binding to human CD47 (FIG. 5B), with the scFv-Fc fusion form of the identical v-domains being higher potency than the IgG.

FIG. 6A-FIG. 6H. Direct titration ELISA for designer lead IgGs binding to human and cyno CD47-Fc proteins. Chimeric anti-CD47 (m5F9G4) and designer clones in human IgG1null format were titrated (in μg/ml) in a direct binding ELISA against human and cyno CD47-Fc proteins. The m5F9G4 and library-derived clones demonstrated binding activity against both orthologs of CD47: VH1.1-VH1.3 families versus human (FIG. 6A) and cyno (FIG. 6B); VH1.4-VH1.6 families versus human (FIG. 6C) and cyno (FIG. 6D); VH1.7, VH1.9 and VH1.10 families against human (FIG. 6E) and cyno (FIG. 6F); and VH1.8 family against human (FIG. 6G) and cyno (FIG. 6H).

FIG. 7A-FIG. 7B. ELISA-based CD47-Fc-SIRPα competition assay for VH1.8 family designer leads. ELISA binding signal for human (FIG. 7A) and cyno (FIG. 7B); CD47-Fc proteins to plate-bound human SIRPαwas examined in the presence of titrated competitor IgGs in IgG1null format, plus m5F9G4 and h5F9G4 in IgG1null format as positive controls.

FIG. 8. ELISA-based CD47-Fc-SIRPα competition assay for further designer leads. ELISA binding signal for human CD47-Fc to plate-bound human SIRPo was examined in the presence of titrated competitor IgGs in IgG1null format, plus Isotype IgG1 and m5F9G4 in IgG1null format.

FIG. 9A-FIG. 9D. Assay technology known under the trademark HTRF®-based solution-phase, high-sensitivity, CD47 epitope competition assay for designer leads. Assay technology known under the trademark HTRF® binding signal for the h5F9G4 IgG to human or cyno CD47 was examined in the presence of titrated competitor IgGs including library-derived leads, plus unlabelled h5F9G4 and m5F9G4 as positive controls. All VH1.8-family designer leads exhibited potent concentration-dependent inhibition of h5F9G4 binding to human (FIG. 9A) and cyno (FIG. 9B) CD47. Other selected designer leads also exhibited concentration-dependent inhibition of h5F9G4 binding to human (FIG. 9C) and cyno (FIG. 20) 9D) CD47, with reduced potency.

FIG. 10A-FIG. 10D. Flow cytometric binding to HL60, human and cyno CD47+CHO-K1 cells for designer leads. Anti-CD47 controls m5F9G4 and h5F9G4, designer leads and Isotype control (all IgG1null) were examined for specific binding on wild type (WT, i.e. untransfected) CHO-K1 cells (FIG. 10A), human CD47-transfected CHO-K1 cells (FIG. 10C), cyno CD47-transfected CHO-K1 cells (FIG. 10B), and the human AML cell line HL60 (FIG. 10D). IgGs were tested at concentrations ranging from 24-100,000 ng/ml. Concentration-dependent binding was observed against human and cyno cell lines, plus HL60 cells, for all CD47-specific antibodies but not isotype controls. No binding signals above background were observed against wild type CHO-K1 cells.

FIG. 11A-FIG. 11C. Development risk ELISAs. This assay showed that designer leads, h5F9G4 and m5F9G4 antibodies in IgG1 form exhibit equivalent or lower binding (with the exception of VH1.12/VL8) to the negatively charged biomolecules Insulin (FIG. 11A), double-stranded DNA (dsDNA) (FIG. 11B) and single-stranded DNA (ssDNA) (FIG. 11C) than the negative control IgG1 Ustekinumab analogue. Strong off-target binding to these molecules, as observed for Bococizumab and Briakinumab analogues has been shown to be a high-risk indicator of poor pharmacokinetics of therapeutic antibodies.

FIG. 12A-FIG. 12C. Summary analysis of the VH and VL sequences of h5F9G4 using immunogenicity assessment tool known under the trademark iTope™. Peptides spanning the full v-domain sequences were tested as 9mer peptides in one amino acid increments. Each row indicates the position of a p1 anchor residue of a 9mer peptide which is scored for MHC class II binding (core 9mer). Each box indicates a binding score of >0.6 between the aligned 9mer and MHC class II. A promiscuous binding peptide is defined as binding 17 or more alleles. Regions containing potentially immunogenic peptides are indicated in ‘Promiscuous High’ (HAF) and ‘Promiscuous Moderate’ (LAF) columns; dark grey vertical bars indicate HAF MHC class II binding peptides and light grey vertical bars indicate LAF MHC class II binding peptides. In the ‘TCED’ column, regions representing closely homologous peptides from the T cell epitope database are shown as vertical grey bars. Example analyses are shown for the key HAF and TCED+epitopes found in the LCDR1 (SEQ ID NO: 70) (FIG. 12A), HCDR1 (SEQ ID NO: 71) (FIG. 12B) and HCDR3 (SEQ ID NO: 72) (FIG. 12C).

FIG. 13A-FIG. 13B. Summary analysis of the VL sequences of key leads using immunogenicity assessment tool known under the trademark iTope™. Peptides spanning the full v-domain sequences were tested as 9mer peptides in one amino acid increments across the full v-domains (VL1.1 shown here as an example). Data is represented as in FIG. 13A-FIG. 13B. Example analyses are shown for the key HAF and TCED+ epitopes found in the LCDR2 (SEQ ID NO: 73) (FIG. 13A) and LCDR3 (SEQ ID NO: 74) (FIG. 13B).

FIG. 14A-FIG. 14C. Phagocytosis potency assays using human macrophage and Jurkat cells. Clones h5F9G4, VH1.8/VL1.1, VH1.8/VL1.6, and VH1.8/VL1.6-DI in IgG4 (S228P) format were examined for their relative potencies in a flow cytometric assay of phagocytosis, using human monocyte-derived macrophages derived from 3 separate donors and Jurkat human cancer cells as the CD47+ target cells. Phagocytosis was measured as Percentage CFSE+ cells from CD11b+ population for Donors 1 (FIG. 14A), 2 (FIG. 14B) and 3 (FIG. 14C).

### DETAILED DESCRIPTION OF THE INVENTION

According to a first aspect of the invention, there is provided an antibody molecule which specifically binds to human CD47 and optionally also to cynomolgus monkey CD47, or an antigen-binding portion thereof, wherein the antibody molecule or antigen-binding portion comprises a heavy chain variable region with:

In the IGHV5-51 background; an HCDR1 having amino acids in sequence in the following order: G-Y-S-F-T-N or a conservative substitution of N (such as S)-Y-N or a conservative substitution of N (such as S)-M or a conservative substitution of M (such as I)-H or any amino acid (such as G) (SEQ ID NO:28);
an HCDR2 having amino acids in sequence in the following order: M or a conservative substitution of M (for example, I)-G-T or any amino acid (such as I)—I—Y-P-G-N or any amino acid (such as D)-D or any amino acid (such as S)-D-T-S or any amino acid (such as R)—Y—N or any amino acid (such as S, H)-Q or any amino acid (such as P)-K or any amino acid (such as S)-F-Q-G (SEQ ID NO:29); and
an HCDR3 having amino acids in sequence in the following order: G or any amino acid (such as Q)-G-Y or any amino acid (such as A, C, D, E, F, G, H, I, K, L, M, N, Q, R, S, T, V, W)-R-A or any amino acid (such as I, T, V, W)-M or any amino acid (such as A, E, K, L, P, Q, R, S, T, V, W)-D-Y (SEQ ID NO:30).

In some aspects an anti-CD47 antibody or antigen-binding portion provided herein specifically binds to a CD47 protein comprising or consisting of SEQ ID NO:24 or SEQ ID NO: 25. In some aspects an anti-CD47 antibody or antigen-binding portion provided herein specifically binds to a CD47 protein having an amino acid sequence that is at least about 90%, at least about 91%, at least about 92%, at least about 93%, at least about 94%, at least about 95%, at least about 96%, at least about 97%, at least about 98% or at least about 99% identical to SEQ ID NO:24 or SEQ ID NO:25.

In the IGHV1-3 background; an HCDR1 having amino acids in sequence in the following order: G-Y-T-F-T-N or any amino acid (such as D)-Y-N or any amino acid (such as A)-M-H (SEQ ID NO:31);
an HCDR2 having amino acids in sequence in the following order: M-G-T-I-Y-P-G-N-D-D or any amino acid (such as N)-T-S or any amino acid (such as K)-Y-N or any amino acid (such
as S)-Q-K-F-Q-G (SEQ ID NO:32); and an HCDR3 having amino acids in sequence in the following order: G or any amino acid (such as Q)-G-Y or any amino acid (such as A, C, D, E, F, G, H, I, K, L, M, N, Q, R, S, T, V, W)-R-A or any amino acid (such as I, T, V, W)-M or any amino acid (such as A, E, K, L, P, Q, R, S, T, V, W)-D-Y (SEQ ID NO:30).

In aspects of the invention, the HCDR1 of the antibody molecule or antigen-binding portion may exclude the sequence GYTFTNYNMH (SEQ ID NO:33; 5F9G4 murine/humanized antibody HCDR1 disclosed in WO2011143624A2; US20130142786A1), the HCDR2 of the antibody molecule or antigen-binding portion may exclude the sequence MGTIYPGNDDTSYNQKFKD (SEQ ID NO:34; 5F9G4 murine/humanized antibody HCDR1 disclosed in WO2011143624A2; US20130142786A1), and/or the HCDR3 of the antibody molecule or antigen-binding portion may exclude the sequence GGYRAMDY (SEQ ID NO: 35; 5F9G4 murine/humanized antibody HCDR3 disclosed in WO2011143624A2; US20130142786A1).

The antibody molecule or antigen-binding portion may further comprise a light chain variable region with:
an LCDR1 having amino acids in sequence in the following order: R-S-S-Q-S-I or a conservative substitution of I (such as L)-V or a conservative substitution of V (such as L)-Y or a conservative substitution of Y (such as H)-S-N or any amino acid (such as G, S, K)-G or any amino acid (such as A, Y)-N or any amino acid (such as Q, Y)-T or any amino acid (such as N)-Y-L-G or any amino acid (such as D) (SEQ ID NO:36);
an LCDR2 having amino acids in sequence in the following order: K-V or any amino acid (such as G)-S-N-R-F or any amino acid (such as A, S)-S(SEQ ID NO:37); and
an LCDR3 having amino acids in sequence in the following order: F or any amino acid (such as L, M)-Q-G or any amino acid (such as A)-S or any amino acid (such as T, L, M)-H or any amino acid (such as Q, R)-V or any amino acid (such as I, T)-P-Y-T or any amino acid (such as I) (SEQ ID NO:38).

In aspects of the invention, the LCDR1 of the antibody molecule or antigen-binding portion may exclude the s RSSQSIVYSNGNTYLG (SEQ ID NO:39; 5F9G4 murine/humanized antibody LCDR1 disclosed in WO2011143624A2; US20130142786A1), and/or the LCDR2 of the antibody molecule or antigen-binding portion may exclude the sequence KVSNRFS (SEQ ID NO:40; 5F9G4 murine/humanized antibody LCDR2 disclosed in WO2011143624A2; US20130142786A1), and/or the LCDR3 of the antibody molecule or antigen binding portion may exclude the sequence FQGSHVPYT (SEQ ID NO:41).

In some aspects, disclosed herein is an anti-CD47 antibody or an antigen-binding portion thereof, wherein the antibody comprises a heavy chain variable (VH) region and a light chain variable (VL) region, wherein
(a) the HCDR1 comprises the amino acid sequence G-Y-X₁-F-T-X₂-Y-X₃-X₄-X₅, wherein X₁ is S or a conservative substitution of S, X₂ is N or a conservative substitution of N (for example, S), X₃ is N or any other amino acid (for example, S, A), X₄ is M or a conservative substitution of M (for example, I) and X₅ is H or any other amino acid (for example, G) (SEQ ID NO:42);
(b) the HCDR2 comprises the amino acid sequence X₁-X₂-X₃-I-Y-P-G-X₄-X₅-X₆-T-X₇-Y-X₈-X₉-X₁₀-F-Q-G, wherein X₁ is M or a conservative substitution of M (for example, I), X₂ is G or a conservative substitution of G, X₃ is T or any other amino acid (for example, I), X₄ is N or any other amino acid (for example, D), X₅ is D or any other amino acid (for example, S), X₆ is D or any other amino acid, X₇ is S or any other amino acid (for example, R), X₈ is N or any other amino acid (for example, S), X₉ is Q or any other amino acid (for example, P) and X₁₀ is K or any other amino acid (for example, S) (SEQ ID NO:75);
(c) the HCDR3 comprises the amino acid sequence X₁-G-X₂-R-X₃-X₄-D-Y, wherein X₁ is G or any other amino acid (for example, Q), X₂ is Y or any other amino acid (for example, A, C, D, E, F, G, H, I, K, L, M, N, Q, R, S, T, V or W), X₃ is A or any other amino acid (for example, I, T, V or W) and X₄ is M or any other amino acid (for example, A, E, K, L, P, Q, R, S, T, V or W) (SEQ ID NO:44);
(d) the LCDR1 comprises the amino acid sequence R—S-S-Q-S-X₁-X₂-X₃-S-X₄-X₅-X₆-X₇-Y-L-X₈, wherein X₁ is I or a conservative substitution of I (for example, L), X₂ is V or a conservative substitution of V (for example, L), X₃ is Y or a conservative substitution of Y (for example, H), X₄ is N or any other amino acid (for example, G, S or K), X₅ is G or any other amino acid (for example, A or Y), X₆ is N or any other amino acid (for example, Q or Y), X₇ is T or any other amino acid (for example, N) and X₈ is G or any other amino acid (for example, D) (SEQ ID NO:45);
(e) the LCDR2 comprises the amino acid sequence K-X₁—S—N-R-X₂-S, wherein X₁ is V or any other amino acid (for example, G) and X₂ is F or any other amino acid (for example, A or S) (SEQ ID NO:46); and
(f) the LCDR3 comprises the amino acid sequence X₁-Q-X₂-X₃-X₄-X₅-P-Y-X₆, wherein X₁ is F or any other amino acid (for example, L or M), X₂ is G or any other amino acid (for example, A), X₃ is S or any other amino acid (for example, T, L or M), X₄ is H or any other amino acid (for example, Q or R), X₅ is V or any other amino acid (for example, I or T), X₆ is T or any other amino acid (for example, I).

In some aspects, disclosed herein is an anti-CD47 antibody or an antigen-binding portion thereof, wherein the antibody comprises a heavy chain variable (VH) region comprising, in amino-terminal to carboxyl-terminal order, FR1-HCDR1-FR2-HCDR2-FR3-HCDR3-FR4 and a light chain variable (VL) region comprising, in amino-terminal to carboxyl-terminal order, FR1-LCDR1-FR2-LCDR2-FR3-LCDR3-FR4, wherein the HCDR1 is SEQ ID NO:42, the HCDR2 is SEQ ID NO:75, the HCDR3 is SEQ ID NO:44, the LCDR1 is SEQ ID NO:45, the LCDR2 is SEQ ID NO:46 and the LCDR3 is X₁-Q-X₂-X₃-X₄-X₅-P-Y-X₆, wherein X₁ is F or any other amino acid (for example, L or M), X₂ is G or any other amino acid (for example, A), X₃ is S or any other amino acid (for example, T, L or M), X₄ is H or any other amino acid (for example, Q or R), X₅ is V or any other amino acid (for example, I or T), X₆ is T or any other amino acid (for example, I), wherein the heavy chain FR1, FR2, FR3 and FR4 amino acid sequences are the heavy chain FR1, FR2, FR3 and FR4 amino acid sequences in SEQ ID NO:141 (see Table 2) and wherein the light chain FR1, FR2, FR3 and FR4 amino acid sequences are the light chain FR1, FR2, FR3 and FR4 amino acid sequences in SEQ ID NO:144 (see Table 2).

As elaborated herein, the present inventors have succeeded for the first time in generating a number of optimized anti-CD47 antibody molecules using CDR sequences derived from the murine anti-CD47 antibody 5F9G4 disclosed in WO2011143624A2; US20130142786A1. In embodiments of the present invention, these antibody molecules have been selected to have binding specificity to both human CD47 as well as cynomolgus monkey CD47 (to facilitate in vivo studies in an appropriate animal test species). Further refining of the optimized antibody molecules as described herein has provided improved variable domain stability, higher expression yields, and/or reduced immunogenicity.

Preferred optimized anti-CD47 antibody molecules of the present invention do not necessarily have the maximum number of human germline substitutions at corresponding murine CDR or other (such as framework) amino acid positions. As elaborated in the experimental section below, we have found that “maximally humanized” antibody molecules are not necessary “maximally optimized” in terms of anti-CD47 binding characteristics and/or other desirable features.

The present invention encompasses modifications to the amino acid sequence of the antibody molecule or antigen-binding portion thereof as defined herein. For example, the invention includes antibody molecules and corresponding antigen-binding portions thereof comprising functionally equivalent variable regions and CDRs which do not significantly affect their properties as well as variants which have enhanced or decreased activity and/or affinity. For example, the amino acid sequence may be mutated to obtain an antibody with the desired binding affinity to CD47. Insertions which include amino- and/or carboxyl-terminal fusions ranging in length from one residue to polypeptides containing a hundred or more residues, as well as intrasequence insertions of single or multiple amino acid residues, are envisaged. Examples of terminal insertions include an antibody molecule with an N-terminal methionyl residue or the antibody molecule fused to an epitope tag. Other insertional variants of the antibody molecule include the fusion to the N- or C-terminus of the antibody of an enzyme or a polypeptide which increases the half-life of the antibody in the blood circulation.

The antibody molecule or antigen-binding portion of the invention may include glycosylated and nonglycosylated polypeptides, as well as polypeptides with other post-translational modifications, such as, for example, glycosylation with different sugars, acetylation, and phosphorylation. The antibody molecule or antigen-binding portion of the invention may be mutated to alter such post-translational modifications, for example by adding, removing or replacing one or more amino acid residues to form or remove a glycosylation site.

The antibody molecule or antigen-binding portion of the invention may be modified for example by amino acid substitution to remove potential proteolytic sites in the antibody.

In the antibody molecule or antigen-binding portion thereof, the HCDR1 may have the amino acid sequence: G-Y-T/S-F-T-N/S/D-Y-N/S/A-M/I-H/G (SEQ ID NO:76); the HCDR2 may have the amino acid sequence: M/I/V-G/A/S-T/I-I-Y-P-G-N/D-D/S-D/N-T-S/K/R-Y-N/S/H-Q/P/H-K/S-F-K/Q-D/G (SEQ ID NO:77); and the HCDR3 may have the amino acid sequence: G/Q-G-Y/A/C/D/E/F/G/H/I/K/L/M/N/Q/R/S/T/V/W-R-A/I/T/V/W-M/A/E/K/L/P/Q/R/S/T/V/W-D-Y (SEQ ID NO:78).

For example, the HCDR1 may have the amino acid sequence: G-Y-T/S-F-T-N/D-Y-N/A-M/I-H (SEQ ID NO:79); the HCDR2 may have the amino acid sequence: M-G-T-I-Y-P-G-N/D-D/S-D/N-T-S/K/R-Y-N/S-Q/P/H-K/S-F-Q-G (SEQ ID NO:80); and the HCDR3 may have the amino acid sequence: G-G-Y/E/F/I/K//N/V-R-A-M/E/Q-D-Y (SEQ ID NO:81).

In the antibody molecule or antigen-binding portion thereof, the LCDR1 may have the amino acid sequence: R-S-S-Q-S-L-V/L-Y/H-S-N/G/S/K-G/A/Y-N/Y-T/N-Y-L-G/D (SEQ ID NO:82); the LCDR2 may have the amino acid sequence: K-G-S-N-R-F/A-S(SEQ ID NO:83); and the LCDR3 may have the amino acid sequence: F/M-Q-G/A-S-H/Q-V-P-Y-T/I (SEQ ID NO:84).

For example, the LCDR1 may have the amino acid sequence: R-S-S-Q-S-L-L-H-S-N/K-G/A/Y-N/Q/Y-T/N-Y-L-G (SEQ ID NO:85); the LCDR2 may have the amino acid sequence: K-V/G-S-N-R-F/A/S-S(SEQ ID NO:86); and the LCDR3 may have the amino acid sequence: F/L/M-Q-G/A-S/T/L/M-H/Q/R-V/I/T-P-Y-T/I.

In specific embodiments of the invention, the antibody molecule or antigen-binding portion may comprise:
(a) the amino acid sequences GYTFTNYAMH (SEQ ID NO:48; HCDR1), MGTIYPGNDDTKYNQKFQG (SEQ ID NO:49; HCDR2), GGFRAMDY (SEQ ID NO:50; HCDR3), RSSQSLLHSNGNTYLG (SEQ ID NO:51; LCDR1), KGSNRFS (SEQ ID NO:52; LCDR2), FQASHVPYT (SEQ ID NO:53; LCDR3), [Clone VH1.8/VL1.1]; or 35
(b) the amino acid sequences GYTFTNYAMH (SEQ ID NO:48; HCDR1), MGTIYPGNDDTKYNQKFQG (SEQ ID NO:49; HCDR2), GGFRAMDY (SEQ ID NO:50; HCDR3), RSSQSLLHSNGNTYLG (SEQ ID NO:51; LCDR1), KGSNRFS (SEQ ID NO:52; LCDR2), FQASQVPYT (SEQ ID NO:54; LCDR3), [Clone VH1.8/VL1.2]; or
(c) the amino acid sequences GYTFTNYAMH (SEQ ID NO:48; HCDR1), MGTIYPGNDDTKYNQKFQG (SEQ ID NO:49; HCDR2), GGFRAMDY (SEQ ID NO:50; HCDR3), RSSQSLLHSNANTYLG (SEQ ID NO:55; LCDR1), KGSNRFS (SEQ ID NO:52; LCDR2), FQASQVPYT (SEQ ID NO:54; LCDR3), [Clone VH1.8/VL1.3]; or
(d) the amino acid sequences GYTFTNYAMH (SEQ ID NO:48; HCDR1), MGTIYPGNDDTKYNQKFQG (SEQ ID NO:49; HCDR2), GGFRAMDY (SEQ ID NO:50; HCDR3), RSSQSLLHSNAYNYLG (SEQ ID NO:56; LCDR1), KGSNRFS (SEQ ID NO:52; LCDR2), FQASQVPYT (SEQ ID NO:54; LCDR3), [Clone VH1.8/VL1.4]; or
(e) the amino acid sequences GYTFTNYAMH (SEQ ID NO:48; HCDR1), MGTIYPGNDDTKYNQKFQG (SEQ ID NO:49; HCDR2), GGFRAMDY (SEQ ID NO:50; HCDR3), RSSQSLLHSNAYNYLG (SEQ ID NO:56; LCDR1), KGSNRFS (SEQ ID NO:52; LCDR2), FQASHVPYT (SEQ ID NO:53; LCDR3), [Clone VH1.8/VL1.5]; or
f) the amino acid sequences GYTFTNYAMH (SEQ ID NO:48; HCDR1), MGTIYPGNDDTKYNQKFQG (SEQ ID NO:49; HCDR2), GGFRAMDY (SEQ ID NO:50; HCDR3), RSSQSLLHSNANTYLG (SEQ ID NO:55; LCDR1), KGSNRFS (SEQ ID NO:52; LCDR2), FQASHVPYT (SEQ ID NO:53; LCDR3), [Clone VH1.8/VL1.6]; or
(g) the amino acid sequences GYTFTNYAMH (SEQ ID NO:48; HCDR1), MGTIYPGNDDTKYNQKFQG (SEQ ID NO:49; HCDR2), GGYRAEDY (SEQ ID NO:59; HCDR3), RSSQSLLHSNGNTYLG (SEQ ID NO:51; LCDR1), KGSNRFS (SEQ ID NO:52; LCDR2), FQASHVPYT (SEQ ID NO:53; LCDR3), [Clone VH1.1/VL1.1]; or
(h) the amino acid sequences GYTFTNYAMH (SEQ ID NO:48; HCDR1), MGTIYPGNDDTKYNQKFQG (SEQ ID NO:49; HCDR2), GGYRAEDY (SEQ ID NO:59; HCDR3), RSSQSLLHSNGNTYLG (SEQ ID NO:51; LCDR1), KGSNRFS (SEQ ID NO:52; 25 LCDR2), FQASQVPYT (SEQ ID NO:54; LCDR3), [Clone VH1.1/VL1.2]; or
(i) the amino acid sequences GYTFTNYAMH (SEQ ID NO:48; HCDR1), MGTIYPGNDDTKYNQKFQG (SEQ ID NO:49; HCDR2), GGYRAEDY (SEQ ID NO:59; HCDR3), RSSQSLLHSNANTYLG (SEQ ID NO:55; LCDR1), KGSNRFS (SEQ ID NO:52; LCDR2), FQASQVPYT (SEQ ID NO:54; LCDR3), [Clone VH1.1/VL1.3]; or 30)
(j) the amino acid sequences S GYTFTNYAMH (SEQ ID NO:48; HCDR1), MGTIYPGNDDTKYNQKFQG (SEQ ID NO:49; HCDR2), GGYRAEDY (SEQ ID NO:59; HCDR3), RSSQSLLHSNANTYLG (SEQ ID NO:55; LCDR1), KGSNRFS (SEQ ID NO:52; LCDR2), FQASHVPYT (SEQ ID NO:53; LCDR3), [Clone VH1.1/VL1.6]; or
(k) the amino acid sequences GYTFTDYNMH (SEQ ID NO:88; HCDR1), MGTIYPGNDDTKYNQKFQG (SEQ ID NO:49; HCDR2), GGFRAMDY (SEQ ID NO:50; HCDR3), RSSQSLLHSNGNTYLG (SEQ ID NO:51; LCDR1), KGSNRFS (SEQ ID NO:52; LCDR2), FQASHVPYT (SEQ ID NO:53; LCDR3), [Clone VH1.5/VL1.1]; or
(l) the amino acid sequences GYTFTNYNIH (SEQ ID NO:89; HCDR1), MGTIYPGNDDTSYSQKFQG (SEQ ID NO:90; HCDR2), GGFRAMDY (SEQ ID NO:50; HCDR3), RSSQSLLHSNGNTYLG (SEQ ID NO:51; LCDR1), KGSNRFS (SEQ ID NO:52; LCDR2), FQASHVPYT (SEQ ID NO:53; LCDR3), [Clone VH1.7/VL1.1]; or
(m) the amino acid sequences GYTFTNYNIH (SEQ ID NO:89; HCDR1), MGTIYPGNDDTSYSQKFQG (SEQ ID NO:90; HCDR2), GGFRAMDY (SEQ ID NO:50; HCDR3), RSSQSLLHSNGNTYLG (SEQ ID NO:51; LCDR1), KGSNRFS (SEQ ID NO:52; 10) LCDR2), FQASQVPYT (SEQ ID NO:54; LCDR3), [Clone VH1.7/VL1.2]; or
(n) the amino acid sequences GYTFTNYNIH (SEQ ID NO:89; HCDR1), MGTIYPGNDDTSYSQKFQG (SEQ ID NO:90; HCDR2), GGFRAMDY (SEQ ID NO:50; HCDR3), RSSQSLLHSNANTYLG (SEQ ID NO:55; LCDR1), KGSNRFS (SEQ ID NO:52; LCDR2), FQASQVPYT (SEQ ID NO:54; LCDR3), [Clone VH1.7/VL1.3]; or
(o) the amino acid sequences GYTFTNYNIH (SEQ ID NO:89; HCDR1), MGTIYPGNDDTSYSQKFQG (SEQ ID NO:90; HCDR2), GGFRAMDY (SEQ ID NO:50; HCDR3), RSSQSLLHSNAYNYLG (SEQ ID NO:56; LCDR1), KGSNRFS (SEQ ID NO:52; LCDR2), FQASHVPYT (SEQ ID NO:53; LCDR3), [Clone VH1.7/VL1.5]; or
(p) the amino acid sequences GYTFTNYNIH (SEQ ID NO:89; HCDR1), MGTIYPGNDDTSYSQKFQG (SEQ ID NO:90; HCDR2), GGFRAMDY (SEQ ID NO:50; HCDR3), RSSQSLLHSNANTYLG (SEQ ID NO:55; LCDR1), KGSNRFS (SEQ ID NO:52; LCDR2), FQASHVPYT (SEQ ID NO:53; LCDR3), [Clone VH1.7/VL1.6]; or
(q) the amino acid sequences GYTFTNYAMH (SEQ ID NO:48; HCDR1), MGTIYPGNDDTSYSQKFQG (SEQ ID NO:90; HCDR2), GGYRAEDY (SEQ ID NO:59; HCDR3), RSSQSLLHSNGNTYLG (SEQ ID NO:51; LCDR1), KGSNRFS (SEQ ID NO:52; 25 LCDR2), FQASHVPYT (SEQ ID NO:53; LCDR3), [Clone VH1.6/VL1.1]; or
(r) the amino acid sequences GYTFTNYAMH (SEQ ID NO:48; HCDR1), MGTIYPGNDDTSYSQKFQG (SEQ ID NO:90; HCDR2), GGYRAEDY (SEQ ID NO:59; HCDR3), RSSQSLLHSNGNTYLG (SEQ ID NO:51; LCDR1), KGSNRFS (SEQ ID NO:52; LCDR2), FQASQVPYT (SEQ ID NO:54; LCDR3), [Clone VH1.6/VL1.2]; or
(s) the amino acid sequences GYTFTNYAMH (SEQ ID NO:48; HCDR1), MGTIYPGNDDTSYSQKFQG (SEQ ID NO:90; HCDR2), GGYRAEDY (SEQ ID NO:59; HCDR3), RSSQSLLHSNGNTYLG (SEQ ID NO:51; LCDR1), KGSNRFS (SEQ ID NO:52; LCDR2), FQASHVPYT (SEQ ID NO:53; LCDR3), [Clone VH1.9/VL1.1]; or
(t) the amino acid sequences GYTFTNYAMH (SEQ ID NO:48; HCDR1), MGTIYPGNDDTKYSQKFQG (SEQ ID NO:91; HCDR2), GGYRAEDY (SEQ ID NO:59; HCDR3), RSSQSLLHSNGNTYLG (SEQ ID NO:51; LCDR1), KGSNRFS (SEQ ID NO:52; LCDR2), FQASQVPYT (SEQ ID NO:54; LCDR3), [Clone VH1.9/VL1.2]; or
(u) the amino acid sequences GYSFTNYNIH (SEQ ID NO:57; HCDR1), MGTIYPGNSDTSYNPSFQG (SEQ ID NO:61; HCDR2), GGVRAMDY (SEQ ID NO:62; HCDR3), RSSQSLLHSNGNTYLG (SEQ ID NO:51; LCDR1), KGSNRFS (SEQ ID NO:52; LCDR2), MQASQVPYT (SEQ ID NO:63; LCDR3), [Clone D6]; or
(v) the amino acid sequences GYSFTNYNIH (SEQ ID NO:57; HCDR1), MGTIYPGNSDTSYNPKFQG (SEQ ID NO:92; HCDR2), GGKRAMDY (SEQ ID NO:93; HCDR3), RSSQSLLHSNGNTYLG (SEQ ID NO:51; LCDR1), KGSNRFS (SEQ ID NO:52; 10) LCDR2), MQASQVPYT (SEQ ID NO:63; LCDR3), [Clone F-E5]; or
(w) the amino acid sequences GYSFTNYNIH (SEQ ID NO:57; HCDR1), MGTIYPGDSDTSYNPKFQG (SEQ ID NO:94; HCDR2), GGYRAQDY (SEQ ID NO:95; HCDR3), RSSQSLLHSNGNTYLG (SEQ ID NO:51; LCDR1), KGSNRFS (SEQ ID NO:52; LCDR2), MQASHVPYT (SEQ ID NO:96; LCDR3), [Clone E-C5]; or
(x) the amino acid sequences GYSFTNYNIH (SEQ ID NO:57; HCDR1), MGTIYPGDSDTSYNPKFQG (SEQ ID NO:94; HCDR2), GGYRAEDY (SEQ ID NO:59; HCDR3), RSSQSLLHSNGNTYLG (SEQ ID NO:51; LCDR1), KGSNRFS (SEQ ID NO:52; LCDR2), MQGSHVPYT (SEQ ID NO:97; LCDR3), [Clone E-D6]; or
(y) the amino sequences GYSFTNYNIH (SEQ ID NO:57; HCDR1), MGTIYPGDSDTRYNQKFQG (SEQ ID NO:98; HCDR2), GGIRAMDY (SEQ ID NO:99; HCDR3), RSSQSLLHSNGNTYLG (SEQ ID NO:51; LCDR1), KGSNRFS (SEQ ID NO:52; LCDR2), FQGSHVPYI (SEQ ID NO:100; LCDR3), [Clone B-A6]; or
(z) the amino acid sequences GYSFTNYNIH (SEQ ID NO:57; HCDR1), MGTIYPGNSDTRYNPKFQG (SEQ ID NO:101; HCDR2), GGFRAMDY (SEQ ID NO:50; HCDR3), RSSQSLLHSNGNTYLG (SEQ ID NO:51; LCDR1), KGSNRFS (SEQ ID NO:52; LCDR2), MQGSHVPYT (SEQ ID NO:97; LCDR3), [Clone D-C12]; or
(zi) the amino acid sequences GYSFTNYNIH (SEQ ID NO:57; HCDR1), MGTIYPGDSDTRYNPKFQG (SEQ ID NO:58; HCDR2), GGYRAEDY (SEQ ID NO:59; HCDR3), RSSQSLLHSNGNTYLG (SEQ ID NO:51; LCDR1), KGSNRFS (SEQ ID NO:52; LCDR2), MQGSHVPYT (SEQ ID NO:97; LCDR3), [Clone E-A1].

In some aspects, disclosed herein is an anti-CD47 antibody or an antigen-binding portion thereof, wherein the antibody comprises a heavy chain variable (VH) region and a light chain variable (VL) region, wherein
(a) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNANTYLG (SEQ ID NO:55), LCDR2 of KGSNRFS (SEQ ID NO:52) and LCDR3 of FQASHVPYT (SEQ ID NO:53);
(b) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNGNTYLG (SEQ ID NO:51), LCDR2 of KGSNRFS (SEQ ID NO:52) and LCDR3 of FQASHVPYT (SEQ ID NO:53);
(c) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNAYNYLG (SEQ ID NO:56), LCDR2 of KGSNRFS (SEQ ID NO:52) and LCDR3 of FQASHVPYT (SEQ ID NO:53);
(d) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNAYNYLG (SEQ ID NO:56), LCDR2 of KGSNRFS (SEQ ID NO:52) and LCDR3 of FQASQVPYT (SEQ ID NO:54);
(e) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNANTYLG (SEQ ID NO:55), LCDR2 of KGSNRFS (SEQ ID NO:52) and LCDR3 of FQASQVPYT (SEQ ID NO:54);
(f) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); and the VL region amino acid sequence comprises LCDR1 of RSSQSLLHSNGNTYLG (SEQ ID NO:51), LCDR2 of KGSNRFS (SEQ ID NO:52) and LCDR3 of FQASQVPYT (SEQ ID NO:54);
(g) the VH region amino acid sequence comprises HCDR1 of GYSFTNYNIH (SEQ ID NO: 57), HCDR2 of MGTIYPGNSDTSYNPSFQG (SEQ ID NO: 61) and HCDR3 of GGVRAMDY (SEQ ID NO:62); and the VL region amino acid sequence comprises LCDR1 of RSSQSLLHSNGNTYLG (SEQ ID NO:51), LCDR2 of KGSNRFS (SEQ ID NO:52) and LCDR3 of MQASQVPYT (SEQ ID NO:63); or
(h) the VH region amino acid sequence comprises HCDR1 of GYSFTNYNIH (SEQ ID NO: 57), HCDR2 of MGTIYPGDSDTRYNPKFQG (SEQ ID NO:58) and HCDR3 of GGYRAEDY (SEQ ID NO:59); and the VL region amino acid sequence comprises LCDR1 of RSSQSLLHSNGNTYLG (SEQ ID NO:51), LCDR2 of KGSNRFS (SEQ ID NO:52) and LCDR3 of MQGSHVPY (SEQ ID NO:60).

In some aspects, disclosed herein is anti-CD47 antibody or an antigen-binding portion thereof, wherein the antibody comprises a heavy chain variable (VH) region and a light chain variable (VL) region, wherein the VH region comprises any one of the VH region amino acid sequences in Table 7 and the VL region comprises any one of the VL region amino acid sequences in Table 7.

In some aspects, disclosed herein is an anti-CD47 antibody or an antigen-binding portion thereof, wherein the antibody comprises a heavy chain variable (VH) region and a light chain variable (VL) region, wherein
(a) the VH region amino acid sequence comprises SEQ ID NO:1 and the VL region amino acid sequence comprises SEQ ID NO:2;
(b) the VH region amino acid sequence comprises SEQ ID NO:3 and the VL region amino acid sequence comprises SEQ ID NO:4;
(c) the VH region amino acid sequence comprises SEQ ID NO:5 and the VL region amino acid sequence comprises SEQ ID NO:6;
(d) the VH region amino acid sequence comprises SEQ ID NO:7 and the VL region amino acid sequence comprises SEQ ID NO:8;
(e) the VH region amino acid sequence comprises SEQ ID NO:9 and the VL region amino acid sequence comprises SEQ ID NO:10;
(f) the VH region amino acid sequence comprises SEQ ID NO:11 and the VL region amino acid sequence comprises SEQ ID NO:12;
(g) the VH region amino acid sequence comprises SEQ ID NO:13 and the VL region amino acid sequence comprises SEQ ID NO:14; or (h) the VH region amino acid sequence comprises SEQ ID NO:15 and the VL region amino acid sequence comprises SEQ ID NO:16.

In some aspects, disclosed herein is an anti-CD47 antibody or an antigen-binding portion thereof, wherein the antibody comprises a heavy chain variable (VH) region and a light chain variable (VL) region, wherein
(a) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 1 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 2;
(b) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 3 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 4;
(c) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 5 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 6;
(d) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 7 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 8;
(e) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 9 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 10;
(f) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 11 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 12; 35
(g) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 13 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO:14; or
(h) the VH region amino acid sequence is at least about 90%, at least about 91%, at least about 92%, at least about 93%, at least about 94%, at least about 95%, at least about 96%, at least about 97%, at least about 98% or at least about 99% identical to SEQ ID NO: 15 and the VL region amino acid sequence is at least about 90%, at least about 91%, at least about 92%, at least about 93%, at least about 94%, at least about 95%, at least about 96%, at least about 97%, at least about 98% or at least about 99% identical to SEQ ID NO:16.

In some aspects, the CDR amino acid sequences of an anti-CD47 antibody are 100% identical to the CDR amino acid sequences in the recited sequences while the FR amino acid sequences are less than 100% identical to the FR amino acid sequences in the recited sequences.

In some aspects, the antibody or antigen-binding portion as defined herein may be isolated.

The antibody molecule or antigen-binding portion as defined herein may cross-compete for binding to CD47 with an antibody or antigen-binding portion thereof comprising the sets of CDRs disclosed herein. In some embodiments, the invention provides an isolated anti-CD47 antibody or an antigen-binding portion thereof, wherein the antibody or antigen-binding portion cross-competes for binding to CD47 with the antibody or antigen-binding portion comprising the sets of CDRs disclosed herein; and (a) comprises fully germline human framework amino acid sequences; and/or (b) does not comprise an ‘NG’ high-risk deamidation site in the LCDR1; (c) does not comprise an ‘NT’ deamidation site in the LCDR1; and/or (d) comprises a human germline peptide sequence with high MHC class II binding affinity in HCDR1 and/or HCDR2; and/or (e) does not comprise a human T cell epitope sequence in the LCDR1; and/or (f) does not comprise a human T cell epitope sequence in the HCDR1 or in the HCDR1/Framework 2 region of the VH domain. In some embodiments, the human germline peptide sequence with high MHC class II binding affinity in the HCDR2 is FQGQVTISA (SEQ ID NO:102) or FQGRVTITA (SEQ ID NO:103).

The terms “cross-compete”, “cross-competition”, “cross-block”, “cross-blocked” and “cross-blocking” are used interchangeably herein to mean the ability of an antibody or portion thereof to interfere with the binding directly or indirectly through allosteric modulation of the anti-CD47 antibodies of the invention to the target CD47 (e.g., human CD47). The extent to which an antibody or portion thereof is able to interfere with the binding of another to the target, and therefore whether it can be said to cross-block or cross-compete according to the invention, can be determined using competition binding assays. One example of a binding competition assay is an assay technology known under the trademark Homogeneous Time Resolved Fluorescence® (HTRF®). One particularly suitable quantitative cross-competition assay uses a FACS-or an AlphaScreen-based approach to measure competition between the labelled (e.g. His tagged, biotinylated or radioactive labelled) antibody or portion thereof and the other antibody or portion thereof in terms of their binding to the target. In general, a cross-competing antibody or portion thereof is, for example, one which will bind to the target in the cross-competition assay such that, during the assay and in the presence of a second antibody or portion thereof, the recorded displacement of the immunoglobulin single variable domain or polypeptide according to the invention is up to 100% (e.g. in a FACS based competition assay) of the maximum theoretical displacement (e.g. displacement by cold (e.g. unlabeled) antibody or fragment thereof that needs to be cross-blocked) by the potentially cross-blocking antibody or fragment thereof that is present in a given amount. Preferably, cross-competing antibodies or portions thereof have a recorded displacement that is between 10% and 100%, or between 50% and 100%.

The antibody molecule or antigen-binding portion as defined herein may comprise one or more substitutions, deletions and/or insertions which remove a post-translational modification (PTM) site, for example a glycosylation site (N-linked or O-linked), a deamination site, a phosphorylation site or an isomerisation/fragmentation site.

More than 350 types of PTM are known. Key forms of PTM include phosphorylation, glycosylation (N- and O-linked), sumoylation, palmitoylation, acetylation, sulfation, myristoylation, prenylation and methylation (of K and R residues). Statistical methods to identify putative amino acid sites responsible for specific PTMs are well known in the art (see Zhou et al., 2016, Nature Protocols 1:1318-1321). Removal of such a site for example by substitution, deletion and/or insertion and then optionally testing (experimentally and/or theoretically) for (a) binding activity and/or (b) loss of the PTM is contemplated.

For example, the 5F9G4 murine LCDR1 (as defined herein, i.e. the amino acid sequence RSSQSIVYSNGNTYLG (SEQ ID NO:39)) has been identified to have a putative ‘high-risk’ deamidation motif at residues 10 and 11 (NG). Removal this site at equivalent positions in an LCDR1 of the invention, for example by substitution of residue 10 (such as to G, S or K), is envisaged (as for example in multiple clones in Table 3). Removal this site at equivalent positions in an LCDR1 of the invention, for example by substitution of residue 11 (such as to A or Y), is also envisaged (as for example in clones containing the VL1.4 v-domain sequence and multiple other clones found in Tables 3 and 4).

In a further example, the 5F9G4 murine LCDR1 (as defined herein, i.e. the amino acid sequence RSSQSIVYSNGNTYLG (SEQ ID NO:39)) has been identified to have a putative ‘high-risk’ deamidation motif at residues 12 and 13 (NT). Removal this risk site at equivalent positions in an LCDR1 of the invention, for example by substitution of residue 12 (such as to Q, Y), is envisaged (as for example in clones containing the VL1.4 v-domain sequence and multiple other clones found in Tables 3 and 4).

The antibody molecule or antigen-binding portion thereof may be human, humanized or chimeric.

The antibody molecule or antigen-binding portion thereof may comprise one or more human variable domain framework scaffolds into which the CDRs have been inserted. For example, the VH region, the VL region, or both the VH and the VL region may comprise one or more human framework region amino acid sequences.

The antibody molecule or antigen-binding portion thereof may comprise an IGHV5-51 human germline scaffold into which the corresponding HCDR sequences have been inserted. The antibody molecule or antigen-binding portion thereof may comprise a VH region that comprises an IGHV5-51 human germline scaffold amino acid sequence into which a set of corresponding HCDR1, HCDR2 and HCDR3 amino acid sequences have been inserted.

The antibody molecule or antigen-binding portion thereof may comprise an IGHV1-3 human germline scaffold into which the corresponding HCDR sequences have been inserted. The antibody molecule or antigen-binding portion thereof may comprise a VH region that comprises an IGHV1-3 human germline scaffold amino acid sequence into which a set of corresponding HCDR1, HCDR2 and HCDR3 amino acid sequences have been inserted.

The antibody molecule or antigen-binding portion thereof may comprise an IGKV2-28 human germline scaffold into which the corresponding LCDR sequences have been inserted. The antibody molecule or antigen-binding portion thereof may comprise a VL region that comprises an IGKV2-28 human germline scaffold amino acid sequence into which a set of corresponding LCDR1, LCDR2 and LCDR3 amino acid sequences have been inserted.

The antibody molecule or antigen-binding portion thereof may comprise an IGHV5-51 human germline scaffold into which the corresponding HCDR sequences have been inserted and an IGKV2-28 human germline scaffold into which the corresponding LCDR sequences have been inserted. The antibody molecule or antigen-binding portion thereof may comprise a VH region that comprises an IGHV5-51 human germline scaffold amino acid sequence into which a set of corresponding HCDR1, HCDR2 and HCDR3 amino acid sequences have been inserted and a VL region that comprises an IGKV2-28 human germline scaffold amino acid sequence into which a set of corresponding LCDR1, LCDR2 and LCDR3 amino acid sequences have been inserted. The HCDR1, HCDR2, HCDR3, LCDR1, LCDR2 and LCDR3 amino acid sequences may be the HCDR1, HCDR2, HCDR3, LCDR1, LCDR2 and LCDR3 amino acid sequences of any one of the clones in Table 4. In some embodiments, all six CDR sequences are from the same clone in Table 4. In some embodiments, the HCDR1, HCDR2 and HCDR3 sequences are from a first clone in Table 4, and the LCDR1, LCDR2 and LCDR3 sequences are from a second clone in Table 4.

The antibody molecule or antigen-binding portion thereof may comprise an IGHV1-3 human germline scaffold into which the corresponding HCDR sequences have been inserted and an IGKV2-28 human germline scaffold into which the corresponding LCDR sequences have been inserted. The antibody molecule or antigen-binding portion thereof may comprise a VH region that comprises an IGHV1-3 human germline scaffold amino acid sequence into which a set of corresponding HCDR1, HCDR2 and HCDR3 amino acid sequences have been inserted and a VL region that comprises an IGKV2-28 human germline scaffold amino acid sequence into which a set of corresponding LCDR1, LCDR2 and LCDR3 amino acid sequences have been inserted. The HCDR1, HCDR2, HCDR3, LCDR1, LCDR2 and LCDR3 amino acid sequences may be the HCDR1, HCDR2, HCDR3, LCDR1, LCDR2 and LCDR3 amino acid sequences of any one of the clones in Table 4. In some embodiments, all six CDR sequences are from the same clone in Table 4. In some embodiments, the HCDR1, HCDR2 and HCDR3 sequences are from a first clone in Table 4, and the LCDR1, LCDR2 and LCDR3 sequences are from a second clone in Table 4.

In some aspects, the antibody molecule or antigen-binding portion thereof may comprise an immunoglobulin constant region. In some embodiments, the immunoglobulin constant region is IgG1, IgG2, IgG3, IgG4, IgA1 or IgA2. In additional embodiments, the immunoglobulin constant region is IgG1, IgG2, IgG3, IgG1null, IgG4 (S228P), IgA1 or IgA2. The antibody molecule or antigen-binding portion thereof may comprise an immunologically inert constant region. In some aspects, an anti-CD47 antibody or antigen-binding portion thereof may comprise an immunoglobulin constant region comprising a wild-type human IgG1 constant region, a human IgG1 constant region comprising the amino acid substitutions L234A, L235A and G237A or a human IgG1 constant region comprising the amino acid substitutions L234A, L235A, G237A and P331S. In some aspects, an anti-CD47 antibody or antigen-binding portion thereof may comprise an immunoglobulin constant region comprising a wild-type human IgG2 constant region or a wild-type human IgG4 constant region. In some aspects, an anti-CD47 antibody may comprise an immunoglobulin constant region comprising any one of the amino acid sequences in Table 8. The Fc region sequences in Table 8 begin at the CH1 domain. In some aspects, an anti-CD47 antibody may comprise an immunoglobulin constant region comprising an amino acid sequence of an Fc region of human IgG4, human IgG4 (S228P), human IgG2, human IgG1, human IgG1-3M or human IgG1-4M. For example, the human IgG4 (S228P) Fc region comprises the following substitution compared to the wild-type human IgG4 Fc region: S228P. For example, the human IgG1-3M Fc region comprises the following substitutions compared to the wild-type human IgG1 Fc region: L234A, L235A and G237A, while the human IgG1-4M Fc region comprises the following substitutions compared to the wild-type human IgG1 Fc region: L234A, L235A, G237A and P331S. In some aspects, a position of an amino acid residue in a constant region of an immunoglobulin molecule is numbered according to EU nomenclature (Ward et al., 1995 Therap. Immunol. 2:77-94). In some aspects, an immunoglobulin constant region may comprise an RDELT (SEQ ID NO:26) motif or an REEM (SEQ ID NO:27) motif (underlined in Table 8). The REEM (SEQ ID NO:27) allotype is found in a smaller human population than the RDELT (SEQ ID NO:26) allotype. In some aspects, an anti-CD47 antibody may comprise an immunoglobulin constant region comprising any one of SEQ ID NOS: 17-23. In some aspects, an anti-CD47 antibody may comprise the six CDR amino acid sequences of any one of the clones in Table 4 and any one of the Fc region amino acid sequences in Table 8. In some aspects, an anti-CD47 antibody may comprise an immunoglobulin heavy chain constant region comprising any one of the Fc region amino acid sequences in Table 8 and an immunoglobulin light chain constant region that is a kappa light chain constant region or a lambda light chain constant region.

In some aspects, disclosed herein is an anti-CD47 antibody or an antigen-binding portion thereof, wherein the antibody comprises a heavy chain variable (VH) region, a light chain variable (VL) region and a heavy chain constant region, wherein
(a) the VH region amino acid sequence comprises HCDR1 of GYTFTNYAMH (SEQ ID NO:48), HCDR2 of MGTIYPGNDDTKYNQKFQG (SEQ ID NO:49) and HCDR3 of GGFRAMDY (SEQ ID NO:50); the VL region amino acid sequence comprises LCDR1 of RSSQSLLHSNANTYLG (SEQ ID NO:55), LCDR2 of KGSNRFS (SEQ ID NO:52) and LCDR3 of FQASHVPYT (SEQ ID NO:53); and the heavy chain constant region comprises any one of SEQ ID NOS: 17-23;

(b) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); the VL region amino acid sequence comprises LCDR1 of RSSQSLLHSNGNTYLG (SEQ ID NO:51), LCDR2 of KGSNRFS (SEQ ID NO:52) and LCDR3 of FQASHVPYT (SEQ ID NO:53); and the heavy chain constant region comprises any one of SEQ ID NOS: 17-23;

(c) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); the VL region amino acid sequence comprises LCDR1 of RSSQSLLHSNAYNYLG (SEQ ID NO:56), LCDR2 of KGSNRFS (SEQ ID NO:52) and LCDR3 of FQASHVPYT (SEQ ID NO:53); and the heavy chain constant region comprises any one of SEQ ID NOS: 17-23;

(d) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); the VL region amino acid sequence comprises LCDR1 of RSSQSLLHSNAYNYLG (SEQ ID NO:56), LCDR2 of KGSNRFS (SEQ ID NO:52) and LCDR3 of FQASQVPYT (SEQ ID NO:54); and the heavy chain constant region comprises any one of SEQ ID NOS: 17-23; 20)
(e) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); the VL region amino acid sequence comprises LCDR1 of RSSQSLLHSNANTYLG (SEQ ID NO:55), LCDR2 of KGSNRFS (SEQ ID NO:52) and LCDR3 of FQASQVPYT (SEQ ID NO:54); and the heavy chain constant region comprises any one of SEQ ID NOS: 17-23;
(f) the VH region amino acid sequence comprises HCDR1 of GYTFTNYAMH (SEQ ID NO: 48), HCDR2 of MGTIYPGNDDTKYNQKFQG (SEQ ID NO:49) and HCDR3 of GGFRAMDY (SEQ ID NO:50); the VL region amino acid sequence comprises LCDR1 of RSSQSLLHSNGNTYLG (SEQ ID NO:51), LCDR2 of KGSNRFS (SEQ ID NO:52) and LCDR3 of FQASQVPYT (SEQ ID NO:54); and the heavy chain constant region comprises any one of SEQ ID NOS: 17-23;
(g) the VH region amino acid sequence comprises HCDR1 of GYSFTNYNIH (SEQ ID NO: 57), HCDR2 of MGTIYPGNSDTSYNPSFQG (SEQ ID NO: 61) and HCDR3 of GGVRAMDY (SEQ ID NO:62); the VL region amino acid sequence comprises LCDR1 of RSSQSLLHSNGNTYLG (SEQ ID NO:51), LCDR2 of KGSNRFS (SEQ ID NO:52) and LCDR3 of MQASQVPYT (SEQ ID NO:63); and the heavy chain constant region comprises any one of SEQ ID NOS: 17-23; or
(h) the VH region amino acid sequence comprises HCDR1 of GYSFTNYNIH (SEQ ID NO: 57), HCDR2 of MGTIYPGDSDTRYNPKFQG (SEQ ID NO:58) and HCDR3 of GGYRAEDY (SEQ ID NO:59); the VL region amino acid sequence comprises LCDR1 of RSSQSLLHSNGNTYLG (SEQ ID NO:51), LCDR2 of KGSNRFS (SEQ ID NO:52) and LCDR3 of MQGSHVPY (SEQ ID NO:60); and the heavy chain constant region comprises any one of SEQ ID NOS: 17-23.

In some aspects, disclosed herein is an anti-CD47 antibody or an antigen-binding portion thereof, wherein the antibody comprises a heavy chain variable (VH) region, a light chain variable (VL) region and a heavy chain constant region, wherein
a) the VH region amino acid sequence comprises or consists of SEQ ID NO:1; the VL region amino acid sequence comprises or consists of SEQ ID NO:2; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A;
(b) the VH region amino acid sequence comprises or consists of SEQ ID NO:3; the VL region amino acid sequence comprises or consists of SEQ ID NO:4; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A;
(c) the VH region amino acid sequence comprises or consists of SEQ ID NO:5; the VL region amino acid sequence comprises or consists of SEQ ID NO:6; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A; 30)
(d) the VH region amino acid sequence comprises or consists of SEQ ID NO:7; the VL region amino acid sequence comprises or consists of SEQ ID NO:8; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A;
(e) the VH region amino acid sequence comprises or consists of SEQ ID NO:9; the VL region amino acid sequence comprises or consists of SEQ ID NO:10; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A;
(f) the VH region amino acid sequence comprises or consists of SEQ ID NO: 11; the VL region amino acid sequence comprises or consists of SEQ ID NO:12; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A;
(g) the VH region amino acid sequence comprises or consists of SEQ ID NO:13; the VL region amino acid sequence comprises or consists of SEQ ID NO:14; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A; or
(h) the VH region amino acid sequence comprises or consists of SEQ ID NO:15; the VL region amino acid sequence comprises or consists of SEQ ID NO:16; and the heavy chain constant region comprises a wild-type human IgG4 constant region, a human IgG4 constant region comprising the amino acid substitution S228P, a wild-type human IgG2 constant region; a wild-type human IgG1 constant region or a human IgG1 constant region comprising the amino acid substitutions L234A, L235A and G237A.

In some aspects, disclosed herein is an anti-CD47 antibody or an antigen-binding portion thereof, wherein the antibody comprises a heavy chain variable (VH) region, a light chain variable (VL) region and a heavy chain constant region, wherein
a) the VH region amino acid sequence comprises or consists of SEQ ID NO:1; the VL region amino acid sequence comprises or consists of SEQ ID NO:2; and the heavy chain constant region comprises any one of SEQ ID NOS: 17-23;
(b) the VH region amino acid sequence comprises or consists of SEQ ID NO:3; the VL region amino acid sequence comprises or consists of SEQ ID NO:4; and the heavy chain constant region comprises any one of SEQ ID NOS: 17-23;
(c) the VH region amino acid sequence comprises or consists of SEQ ID NO:5; the VL region amino acid sequence comprises or consists of SEQ ID NO:6; and the heavy chain constant region comprises any one of SEQ ID NOS: 17-23;
(d) the VH region amino acid sequence comprises or consists of SEQ ID NO:7; the VL region amino acid sequence comprises or consists of SEQ ID NO:8; and the heavy chain constant region comprises any one of SEQ ID NOS: 17-23;
(e) the VH region amino acid sequence comprises or consists of SEQ ID NO:9; the VL region amino acid sequence comprises or consists of SEQ ID NO:10; and the heavy chain constant region comprises any one of SEQ ID NOS: 17-23;
(f) the VH region amino acid sequence comprises or consists of SEQ ID NO: 11; the VL region amino acid sequence comprises or consists of SEQ ID NO: 12; and the heavy chain constant region comprises any one of SEQ ID NOS: 17-23;
(g) the VH region amino acid sequence comprises or consists of SEQ ID NO:13; the VL region amino acid sequence comprises or consists of SEQ ID NO:14; and the heavy chain constant region comprises any one of SEQ ID NOS: 17-23; or
(h) the VH region amino acid sequence comprises or consists of SEQ ID NO: 15; the VL region amino acid sequence comprises or consists of SEQ ID NO:16; and the heavy chain constant region comprises any one of SEQ ID NOS: 17-23.

In some aspects, an anti-CD47 antibody may be immune effector null. In some aspects, an anti-CD47 antibody or an antigen-binding portion thereof does not induce immune effector function and, optionally, suppresses immune effector function. In some aspects, an anti-CD47 may lack measurable binding to human FcγRI, FcγRlla, FcγRIlla and FcγRIllb receptors but maintain binding to human FcγRIlb receptor and optionally maintain binding to human FcRn receptor. FcγRI, FcγRlla, FcγRIlla and FcγRIllb are examples of activating receptors. FcγRIlb is an example of an inhibitory receptor. FcRn is an example of a recycling receptor. In some aspects, binding affinity of an anti-CD47 antibody or an antigen-binding portion thereof for human Fc receptors may be measured by a biosensor system sold under the trademark Biacore® analysis. In some aspects, assay technology known under the trademark Homogeneous Time Resolved Fluorescence (HTRF®) can be used to study binding of an anti-CD47 antibody to human Fc receptors. In one example of the assay technology known under the trademark HTRF®, human IgG1 (wild type) is labelled, as is the full suite of Fc gamma receptors and then antibodies with engineered Fc fragments are used in titration competition. In some aspects, CD47-positive cells may be mixed with human white blood cells and anti-CD47 antibodies, and cell killing by CDC, ADCC and/or ADCP may be measured. In some aspects, an anti-CD47 antibody comprising an amino acid sequence of an Fc region of human IgG1-3M (see Table 8) is effector null. In some aspects, an anti-CD47 antibody comprising an amino acid sequence of an Fc region of human IgG1-3M (see Table 8) is not effector null.

The antibody molecule or antigen-binding portion thereof may be a Fab fragment, a F(ab) 2 fragment, an Fv fragment, an scFv (single-chain Fv) fragment, a tetrameric antibody, a tetravalent antibody, a multispecific antibody (for example, a bispecific antibody), a domain-specific antibody, a single domain antibody, a monoclonal antibody or a fusion protein. In one embodiment, an antibody may be a bispecific antibody that binds specifically to a first antigen and a second antigen, wherein the first antigen is CD47 and the second antigen is not CD47. Antibody molecules and methods for their construction and use are described, in for example Holliger & Hudson (2005, Nature Biotechnol. 23 (9): 1126-1136).

In some aspects, the invention provides a heavy chain variable (VH) region sequence and a light chain variable (VL) region sequence that neutralizes the CD47-SIRPα interaction more potently in the single-chain Fv (scFv) format than in the IgG format. In some embodiments, the VH regions of such molecules are in the IGHV5-51 framework. In some embodiments, the invention encompasses a single-chain Fv (scFv) comprising a heavy chain variable (VH) region and a light chain variable (VL) region, wherein (a) the HCDR1 comprises G-Y-T-F-T-X₁-Y-X₂-X₃-X₄, wherein X₁ is N or a conservative substitution of N, X₂ is N or any other amino acid, X₃ is M or a conservative substitution of M and X₄ is H or any other amino acid (SEQ ID NO: 104); (b) the HCDR2 comprises M-G-X₁-I—Y-P-G-X₂-X₃-X₄-T-X₅-Y-X₆-X₇-X₈-F-Q-G, wherein X₁ is T or any other amino acid, X₂ is N or a 20) conservative substitution of N, X₃ is D or any other amino acid, X₄ is D or any other amino acid, X₅ is S or any other amino acid, X₆ is N or any other amino acid, X₇ is Q or any other amino acid and X₈ is K or any other amino acid (SEQ ID NO:105); (c) the HCDR3 comprises; X₁-G-X₂-R-X₃-X₄-D-Y, wherein X₁ is G or any other amino acid, X₂ is Y or any other amino acid, X₃ is A or any other amino acid and X₄ is M or any other amino acid (SEQ ID NO:44) (d) the LCDR1 comprises R—S-S-Q-S-X₁-X₂-X₃-S-X₄-X₅-X₆-X₇-Y-L-X₈, wherein X₁ is I or a conservative substitution of 1, X₂ is V or a conservative substitution of V, X₃ is Y or a conservative substitution of Y, X₄ is N or any other amino acid, X₅ is G or any other amino acid, X₆ is N or any other amino acid, X₇ is T or any other amino acid and X₈ is G or any other amino acid (SEQ ID NO:45); (e) the LCDR2 comprises K-X₁—S—N-R-X₂-S, wherein X₁ is V or any other amino acid and X₂ is F or any other amino acid (SEQ ID 30) NO: 46); and (f) the LCDR3 comprises X₁-Q-X₂-X₃-X₄-X₅-P-Y-X₆, wherein X₁ is F or any other amino acid, X₂ is G or any other amino acid, X₃ is S or any other amino acid, X₄ is H or any other amino acid, X₅ is V or any other amino acid, X₆ is T or any other amino acid; and wherein the VH region comprises an IGHV5-51 human germline scaffold amino acid sequence into which the HCDR1, HCDR2 and HCDR3 amino acid sequences have been inserted. In one embodiment, an scFv of the invention may comprise (a) the VH amino acid sequence comprises the HCDR1 of GYSFTNYNIH (SEQ ID NO:57), the HCDR2 of MGTIYPGDSDTSYNPKFQG (SEQ ID NO:94) and the HCDR3 of GGYRAEDY (SEQ ID NO: 59); and (b) the VH amino acid sequence comprises the LCDR1 of RSSQSLLHSNGNTYLG (SEQ ID NO:51), the LCDR2 of KGSNRFS (SEQ ID NO:52) and the LCDR3 of MQGSHVPYT (SEQ ID NO:97). In some aspects, an scFv comprises, in amino-terminal to carboxyl-terminal order, VH-VL or VL-VH. In some aspects, an scFv comprises a linker between the VH sequence and the VL sequence. In some embodiments, an scFv further comprises an immunoglobulin constant region or a portion of an immunoglobulin constant region. In some embodiments, the invention provides a multispecific (e.g., bispecific) CD47-binding molecule comprising one or more scFv disclosed herein. In some cases, the immunoglobulin constant region comprises any one of SEQ ID NOS: 17-23.

In another aspect of the invention, there is provided an immunoconjugate comprising the antibody molecule or antigen-binding portion thereof of the invention as defined herein linked to a therapeutic agent.

Examples of suitable therapeutic agents include cytotoxins, radioisotopes, chemotherapeutic agents, immunomodulatory agents, anti-angiogenic agents, antiproliferative agents, pro-apoptotic agents, and cytostatic and cytolytic enzymes (for example RNAses). Further therapeutic agents include a therapeutic nucleic acid, such as a gene encoding an immunomodulatory agent, an anti-angiogenic agent, an anti-proliferative agent, or a pro-apoptotic agent. These drug descriptors are not mutually exclusive, and thus a therapeutic agent may be described using one or more of the above terms.

Examples of suitable therapeutic agents for use in immunoconjugates include the taxanes, maytansines, CC-1065 and the duocarmycins, the calicheamicins and other enediynes, and the auristatins. Other examples include the anti-folates, vinca alkaloids, and the anthracyclines. Plant toxins, other bioactive proteins, enzymes (i.e., Antibody-Directed Enzyme Prodrug Therapy (ADEPT)), radioisotopes, photosensitizers may also be used in immunoconjugates. In addition, conjugates can be made using secondary carriers as the cytotoxic agent, such as liposomes or polymers, Suitable cytotoxins include an agent that inhibits or prevents the function of cells and/or results in destruction of cells. Representative cytotoxins include antibiotics, inhibitors of tubulin polymerization, alkylating agents that bind to and disrupt DNA, and agents that disrupt protein synthesis or the function of essential cellular proteins such as protein kinases, phosphatases, topoisomerases, enzymes, and cyclins.

Representative cytotoxins include, but are not limited to, doxorubicin, daunorubicin, idarubicin, aclarubicin, zorubicin, mitoxantrone, epirubicin, carubicin, nogalamycin, menogaril, pitarubicin, valrubicin, cytarabine, gemcitabine, trifluridine, ancitabine, enocitabine, azacitidine, doxifluhdine, pentostatin, broxuhdine, capecitabine, cladhbine, decitabine, floxuhdine, fludarabine, gougerotin, puromycin, tegafur, tiazofuhn, adhamycin, cisplatin, carboplatin, cyclophosphamide, dacarbazine, vinblastine, vincristine, mitoxantrone, bleomycin, mechlorethamine, prednisone, procarbazine, methotrexate, flurouracils, etoposide, taxol, taxol analogs, platins such as cis-platin and carbo-platin, mitomycin, thiotepa, taxanes, vincristine, daunorubicin, epirubicin, actinomycin, authramycin, azaserines, bleomycins, tamoxifen, idarubicin, dolastatins/auristatins, hemiasterlins, esperamicins and maytansinoids.

Suitable immunomodulatory agents include anti-hormones that block hormone action on tumors and immunosuppressive agents that suppress cytokine production, down-regulate self-antigen expression, or mask MHC antigens.

Also provided is a nucleic acid molecule encoding the antibody molecule or antigen-binding portion thereof of the invention as defined herein. A nucleic acid molecule may encode (a) the VH region amino acid sequence; (b) the VL region amino acid sequence; or (c) both the VH and the VL region amino acid sequences of an anti-CD47 antibody or an antigen-binding portion thereof described herein. In some aspects, the nucleic acid molecule as defined herein may be isolated.

Further provided is a vector comprising the nucleic acid molecule of the invention as defined herein. The vector may be an expression vector.

Also provided is a host cell comprising the nucleic acid molecule or the vector of the invention as defined herein. The host cell may be a recombinant host cell.

In a further aspect there is provided a method of producing an anti-CD47 antibody and/or an antigen-binding portion thereof, comprising culturing the host cell of the invention under conditions that result in expression and/or production of the antibody and/or the antigen-binding portion thereof, and isolating the antibody and/or the antigen-binding portion thereof from the host cell or culture.

In another aspect of the invention there is provided a pharmaceutical composition comprising the antibody molecule or antigen-binding portion thereof of the invention as defined herein, or the nucleic acid molecule of the invention as defined herein, or the vector of the invention as defined herein.

Further provided is a method for enhancing an immune response in a subject, comprising administering to the subject an effective amount of the antibody molecule or antigen-binding portion thereof of the invention as defined herein, or the immunoconjugate of the invention as defined herein, or the nucleic acid molecule of the invention as defined herein, or the vector of the invention as defined herein, or the pharmaceutical composition of the invention as defined herein.

In a further aspect there is provided a method for treating or preventing cancer in a subject, comprising administering to the subject an effective amount of the antibody molecule or antigen-binding portion thereof of the invention as defined herein, or the immunoconjugate of the invention as defined herein, or the nucleic acid molecule of the invention as defined herein, or the vector of the invention as defined herein, or the pharmaceutical composition of the invention as defined herein.

For example, the cancer may be Gastrointestinal Stromal cancer (GIST), pancreatic cancer, melanoma, breast cancer, lung cancer, bronchial cancer, colorectal cancer, prostate cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, or cancer of hematological tissues.

The invention also provides an antibody molecule or antigen-binding portion thereof of the invention as defined herein, or the immunoconjugate of the invention as defined herein, or the nucleic acid molecule of the invention as defined herein, or the vector of the invention as defined herein, or the pharmaceutical composition of the invention as defined herein, for use in the treatment of cancer.

In another aspect the invention provides the antibody molecule, or antigen-binding portion thereof, or the immunoconjugate, or the nucleic acid molecule, or the vector for use, or the method of treatment of the invention as defined herein, for separate, sequential or simultaneous use in a combination combined with a second therapeutic agent, for example an anti-cancer agent.

In a further aspect there is provided the use of an antibody molecule or antigen-binding portion thereof of the invention as defined herein, or an immunoconjugate of the invention as defined herein, or a nucleic acid molecule of the invention as defined herein, or a vector of the invention as defined herein, or a pharmaceutical composition of the invention as defined herein, in the manufacture of a medicament for the treatment of cancer.

The invention also provides a method for treating or preventing an autoimmune disease or an inflammatory disease in a subject, comprising administering to the subject an effective amount of the antibody molecule or antigen-binding portion thereof as defined herein, or the immunoconjugate as defined here, or the nucleic acid molecule as defined herein, or the vector as defined herein, or the pharmaceutical composition as defined herein.

For example, the autoimmune disease or inflammatory disease may be arthritis, asthma, multiple sclerosis, psoriasis, Crohn's disease, inflammatory bowel disease, lupus, Grave's disease and Hashimoto's thyroiditis, or ankylosing spondylitis.

Also provided is an antibody molecule or antigen-binding portion thereof as defined herein, or the immunoconjugate as defined herein, or the nucleic acid molecule as defined herein, or the vector as defined herein, or the pharmaceutical composition as defined herein, for use in the treatment of an autoimmune disease or an inflammatory disease.

Further provided is the use of an antibody molecule or antigen-binding portion thereof as defined herein, or an immunoconjugate as defined herein, or a nucleic acid molecule as defined herein, or a vector as defined herein, or a pharmaceutical composition as defined herein, in the manufacture of a medicament for the treatment of an autoimmune disease or an inflammatory disease.

The invention also provides a method for treating or preventing a cardiovascular disease or a fibrotic disease in a subject, comprising administering to the subject an effective amount of the antibody molecule or antigen-binding portion thereof as defined herein, or the immunoconjugate as defined here, or the nucleic acid molecule as defined herein, or the vector as defined herein, or the pharmaceutical composition as defined herein.

Also provided is an antibody molecule or antigen-binding portion thereof as defined herein, or the immunoconjugate as defined herein, or the nucleic acid molecule as defined herein, or the vector as defined herein, or the pharmaceutical composition as defined herein, for use in the treatment of a cardiovascular disease or a fibrotic disease.

Further provided is the use of an antibody molecule or antigen-binding portion thereof as defined herein, or an immunoconjugate as defined herein, or a nucleic acid molecule as defined herein, or a vector as defined herein, or a pharmaceutical composition as defined herein, in the manufacture of a medicament for the treatment of a cardiovascular disease or a fibrotic disease.

The cardiovascular disease in any aspect of the invention may for example be coronary heart disease or atherosclerosis.

For example, the fibrotic disease in any aspect of the invention may be myocardial infarction, angina, osteoarthritis, pulmonary fibrosis, asthma, cystic fibrosis or bronchitis.

In one embodiment, the invention provides an anti-CD47 antibody or an antigen-binding portion thereof comprising the amino acid sequences disclosed herein for use in therapy.

The pharmaceutical composition of the invention may comprise a pharmaceutically acceptable excipient, carrier or diluent. A pharmaceutically acceptable excipient may be a compound or a combination of compounds entering into a pharmaceutical composition which does not provoke secondary reactions and which allows, for example, facilitation of the administration of the anti-CD47 antibody molecule, an increase in its lifespan and/or in its efficacy in the body or an increase in its solubility in solution. These pharmaceutically acceptable vehicles are well known and will be adapted by the person skilled in the art as a function of the mode of administration of the anti-CD47 antibody molecule.

In some embodiments, the anti-CD47 antibody molecule may be provided in a lyophilised form for reconstitution prior to administration. For example, lyophilised antibody molecules may be re-constituted in sterile water and mixed with saline prior to administration to an individual.

The anti-CD47 antibody molecules will usually be administered in the form of a pharmaceutical composition, which may comprise at least one component in addition to the antibody molecule. Thus pharmaceutical compositions may comprise, in addition to the anti-CD47 antibody molecule, a pharmaceutically acceptable excipient, carrier, buffer, stabilizer or other materials well known to those skilled in the art. Such materials should be non-toxic and should not interfere with the efficacy of the anti-CD47 antibody molecule. The precise nature of the carrier or other material will depend on the route of administration, which may be by bolus, infusion, injection or any other suitable route, as discussed below.

For parenteral, for example sub-cutaneous or intra-venous administration, e.g. by injection, the pharmaceutical composition comprising the anti-CD47 antibody molecule may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringe's Injection, Lactated Ringer's Injection. Preservatives, stabilizers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as a non-ionic surfactant sold under the trademark TWEEN™, a non-ionic surfactant sold under the trademark PLURONICS™ or polyethylene glycol (PEG).

A pharmaceutical composition comprising an anti-CD47 antibody molecule may be administered alone or in combination with other treatments, either simultaneously or sequentially dependent upon the condition to be treated.

An anti-CD47 antibody molecule as described herein may be used in a method of treatment of the human or animal body, including prophylactic or preventative treatment (e.g. treatment before the onset of a condition in an individual to reduce the risk of the condition occurring in the individual; delay its onset; or reduce its severity after onset). The method of treatment may comprise administering the anti-CD47 antibody molecule to an individual in need thereof.

Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Appropriate doses of antibody molecules are well known in the art (Ledermann J. A. et al., 1991, Int. J. Cancer 47:659-664; Bagshawe K. D. et al., 1991, Antibody, Immunoconjugates and Radiopharmaceuticals 4:915-922). Specific dosages may be indicated herein or in the Physician's Desk Reference (2003) as appropriate for the type of medicament being administered may be used. A therapeutically effective amount or suitable dose of an antibody molecule may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g. whole antibody, fragment) and the nature of any detectable label or other molecule attached to the antibody.

A typical antibody dose will be in the range 100 μg to 1 g for systemic applications, and 1 μg to 1 mg for topical applications. An initial higher loading dose, followed by one or more lower doses, may be administered. Typically, the antibody will be a whole antibody, e.g. the IgG1 or IgG4 isotype. This is a dose for a single treatment of an adult patient, which may be proportionally adjusted for children and infants, and also adjusted for other antibody formats in proportion to molecular weight. Treatments may be repeated at daily, twice-weekly, weekly or monthly intervals, at the discretion of the physician. The treatment schedule for an individual may be dependent on the pharmocokinetic and pharmacodynamic properties of the antibody composition, the route of administration and the nature of the condition being treated.

30) Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, anti-CD47 antibody molecules as described herein may be administered as sub-cutaneous injections. Sub-cutaneous injections may be administered using an auto-injector, for example for long or short-term prophylaxis/treatment.

In some preferred embodiments, the therapeutic effect of the anti-CD47 antibody molecule may persist for several multiples of the antibody half-life in serum, depending on the dose. For example, the therapeutic effect of a single dose of the anti-CD47 antibody molecule may persist in an individual for 1 month or more, 2 months or more, 3 months or more, 4 months or more, 5 months or more, or 6 months or more.

The invention also provides a method of producing an antibody molecule which specifically binds to human CD47 and optionally also to cynomolgus monkey CD47 or an antigen-binding portion thereof, comprising the steps of:
(1) grafting anti-CD47 CDRs from a non-human source into a human v-domain framework to produce a humanized anti-CD47 antibody molecule or antigen-binding portion thereof;
(2) generating a phage library of clones of the humanized anti-CD47 antibody molecule or antigen-binding portion thereof comprising one or more mutations in the CDRs;
(3) selecting the phage library for binding to human CD47 and optionally also to cynomolgus monkey CD47;
(4) screening clones from the selection step (3) having binding specificity to human CD47 and optionally also to cynomolgus monkey CD47; and
(5) producing an antibody molecule which specifically binds to human CD47 and optionally also to cynomolgus monkey CD47, or an antigen-binding portion thereof from clones selected from step (4).

The method may comprise a further step of producing additional clones based on the clones selected in step (4), for example based on further exploratory mutagenesis at specific positions in the CDRs of the clones selected in step (4), to enhance humanization and/or minimise human T cell epitope content and/or improve manufacturing properties in the antibody molecule or antigen-binding portion thereof produced in step (5).

Refinements applicable to the above method are as described in Example 1 below.

As used herein, the term “CD47” refers to IAP (Integrin Associated Protein) and variants thereof that retain at least part of the biological activity of CD47. As used herein, CD47 includes all mammalian species of native sequence CD47, including human, rat, mouse and chicken. The term “CD47” is used to include variants, isoforms and species homologs of human CD47. Antibodies of the invention may cross-react with CD47 from species other than human, in particular CD47 from cynomolgus monkey (Macaca fascicularis). Examples of human and cynomolgus CD47 amino acid sequences are provided in Table 9. In certain embodiments, the antibodies may be completely specific for human CD47 and may not exhibit non-human cross-reactivity.

As used herein, an “antagonist” as used in the context of the antibody of the invention or an “anti-CD47 antagonist antibody” (interchangeably termed “anti-CD47 antibody”) refers to an antibody which is able to bind to CD47 and inhibit CD47 biological activity and/or downstream pathway(s) mediated by CD47 signalling. An anti-CD47 antagonist antibody encompasses antibodies that can block, antagonize, suppress or reduce (including significantly) CD47 biological activity, including downstream pathways mediated by CD47 signalling, such as receptor binding and/or elicitation of a cellular response to CD47. For the purposes of the present invention, it will be explicitly understood that the term “anti-CD47 antagonist antibody” encompass all the terms, titles, and functional states and characteristics whereby CD47 itself, and CD47 biological activity (including but not limited to its ability to enhance the activation of phagocytosis by cells of the myeloid lineage), or the consequences of the activity or biological activity, are substantially nullified, decreased, or neutralized in any meaningful degree.

The antibody “specifically binds” “specifically interacts”, “preferentially binds”, “binds” or “interacts” with CD47 if it binds with greater affinity, avidity, more readily and/or with greater duration than it binds to other receptors.

An “antibody molecule” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody molecule” encompasses not only intact polyclonal or monoclonal antibodies, but also any antigen binding fragment (for example, an “antigen-binding portion”) 30 or single chain thereof, fusion proteins comprising an antibody, and any other modified configuration of the immunoglobulin molecule that comprises an antigen recognition site including, for example without limitation, scFv, single domain antibodies (for example, shark and camelid antibodies), maxibodies, minibodies, intrabodies, diabodies, triabodies, tetrabodies, v-NAR and bis-scFv.

An “antibody molecule” encompasses an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), for example IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody molecule, as used herein, refers to one or more fragments of an intact antibody that retain the ability to specifically bind to CD47. Antigen binding functions of an antibody molecule can be performed by fragments of an intact antibody. Examples of binding fragments encompassed within the term “antigen binding portion” of an antibody molecule include Fab; Fab′; F(ab′)2; an Fd fragment consisting of the VH and CH1 domains; an Fv fragment consisting of the VL and VH domains of a single arm of an antibody; a single domain antibody (dAb) fragment, and an isolated complementarity determining region (CDR).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, CH2 and CH3. As is known in the art, an Fc region can be present in dimer or monomeric form.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, and contribute to the formation of the antigen binding site of antibodies. When choosing FR to flank CDRs, for example when humanizing or optimizing an antibody, FRs from antibodies which contain CDR sequences in the same canonical class are preferred.

The CDR definitions used in the present application combine the domains used in the many disparate, often conflicting schemes that have been created in the field, which are based on the combination of immunoglobulin repertoire analyses and structural analyses of antibodies in isolation and in their co-crystals with antigens (see review by Swindells et al., 2016, abYsis: Integrated Antibody Sequence and Structure-Management, Analysis, and Prediction. J Mol Biol. [PMID: 27561707; Epub 22 Aug. 2016]). The CDR definition used herein (a “Unified” definition) incorporates the lessons of all such prior insights and includes all appropriate loop positions required to sample the full residue landscape that potentially mediates target-binding complementarity.

Table 1 shows the amino acid sequences of the 5F9G4 murine anti-CD47 antibody CDRs as defined herein (a “Unified” scheme), in comparison to well-known alternative systems for defining the same CDRs.

As used herein the term “conservative substitution” refers to replacement of an amino acid with another amino acid which does not significantly deleteriously change the functional activity. A preferred example of a “conservative substitution” is the replacement of one amino acid with another amino acid which has a value ≥0 in the following BLOSUM 62 substitution matrix (see Henikoff & Henikoff, 1992, PNAS 89:10915-10919):

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | −1 | −2 | −2 | 0 | −1 | −1 | 0 | −2 | −1 | −1 | −1 | −1 | −2 | −1 | −1 | 0 | −3 | −2 | 0 |
| R | −1 | 5 | 0 | −2 | −3 | 1 | 0 | −2 | 0 | −3 | −2 | 2 | −1 | −3 | −2 | −1 | −1 | −3 | −2 | −3 |
| N | −2 | 0 | 6 | 1 | −3 | 0 | 0 | 0 | 1 | −3 | −3 | 0 | −2 | −3 | −2 | 1 | 0 | −4 | −2 | −3 |
| D | −2 | −2 | 1 | 6 | −3 | 0 | 2 | −1 | −1 | −3 | −4 | −1 | −3 | −3 | −1 | 0 | −1 | −4 | −3 | −3 |
| C | 0 | −3 | −3 | −3 | 9 | −3 | −4 | −3 | −3 | −1 | −1 | −3 | −1 | −2 | −3 | −1 | −1 | −2 | −2 | −1 |
| Q | −1 | 1 | 0 | 0 | −3 | 5 | 2 | −2 | 0 | −3 | −2 | 1 | 0 | −3 | −1 | 0 | −1 | −2 | −1 | −2 |
| E | −1 | 0 | 0 | 2 | −4 | 2 | 5 | −2 | 0 | −3 | −3 | 1 | −2 | −3 | −1 | 0 | −1 | −3 | −2 | −2 |
| G | 0 | −2 | 0 | −1 | −3 | −2 | −2 | 6 | −2 | −4 | −4 | −2 | −3 | −3 | −2 | 0 | −2 | −2 | −3 | −3 |
| H | −2 | 0 | 1 | −1 | −3 | 0 | 0 | −2 | 8 | −3 | −3 | −1 | −2 | −1 | −2 | −1 | −2 | −2 | 2 | −3 |
| I | −1 | −3 | −3 | −3 | −1 | −3 | −3 | −4 | −3 | 4 | 2 | −3 | 1 | 0 | −3 | −2 | −1 | −3 | −1 | 3 |
| L | −1 | −2 | −3 | −4 | −1 | −2 | −3 | −4 | −3 | 2 | 4 | −2 | 2 | 0 | −3 | −2 | −1 | −2 | −1 | 1 |
| K | −1 | 2 | 0 | −1 | −3 | 1 | 1 | −2 | −1 | −3 | −2 | 5 | −1 | −3 | −1 | 0 | −1 | −3 | −2 | −2 |
| M | −1 | −1 | −2 | −3 | −1 | 0 | −2 | −3 | −2 | 1 | 2 | −1 | 5 | 0 | −2 | −1 | −1 | −1 | −1 | 1 |
| F | −2 | −3 | −3 | −3 | −2 | −3 | −3 | −3 | −1 | 0 | 0 | −3 | 0 | 6 | −4 | −2 | −2 | 1 | 3 | −1 |
| P | −1 | −2 | −2 | −1 | −3 | −1 | −1 | −2 | −2 | −3 | −3 | −1 | −2 | −4 | 7 | −1 | −1 | −4 | −3 | −2 |
| S | 1 | −1 | 1 | 0 | −1 | 0 | 0 | 0 | −1 | −2 | −2 | 0 | −1 | −2 | −1 | 4 | 1 | −3 | −2 | −2 |
| T | 0 | −1 | 0 | −1 | −1 | −1 | −1 | −2 | −2 | −1 | −1 | −1 | −1 | −2 | −1 | 1 | 5 | −2 | −2 | 0 |
| W | −3 | −3 | −4 | −4 | −2 | −2 | −3 | −2 | −2 | −3 | −2 | −3 | −1 | 1 | −4 | −3 | −2 | 11 | 2 | −3 |
| Y | −2 | −2 | −2 | −3 | −2 | −1 | −2 | −3 | 2 | −1 | −1 | −2 | −1 | 3 | −3 | −2 | −2 | 2 | 7 | −1 |
| V | 0 | −3 | −3 | −3 | −1 | −2 | −2 | −3 | −3 | 3 | 1 | −2 | 1 | −1 | −2 | −2 | 0 | −3 | −1 | 4. |

The term “monoclonal antibody” (Mab) refers to an antibody, or antigen-binding portion thereof, that is derived from a single copy or clone, including for example any eukaryotic, prokaryotic, or phage clone, and not the method by which it is produced. Preferably, a monoclonal antibody of the invention exists in a homogeneous or substantially homogeneous population.

A “humanized” antibody molecule refers to a form of non-human (for example, murine) antibody molecules, or antigen-binding portion thereof, that are chimeric immunoglobulins, immunoglobulin chains, or fragments thereof (such as Fv, Fab, Fab′, F(ab′)2 or other antigen-binding sub-sequences of antibodies) that contain minimal sequence derived from non-human immunoglobulin. Humanized antibodies may be human immunoglobulins (recipient antibody) in which residues from a CDR of the recipient are replaced by residues from a CDR of a non-human species (donor antibody) such as mouse, rat, or rabbit having the desired specificity, affinity, and capacity.

“Human antibody” or “fully human antibody” refers to an antibody molecule, or antigen-binding portion thereof, derived from transgenic mice carrying human antibody genes or from human cells.

The term “chimeric antibody” is intended to refer to an antibody molecule, or antigen-binding portion thereof, in which the variable region sequences are derived from one species and the constant region sequences are derived from another species, such as an antibody molecule in which the variable region sequences are derived from a mouse antibody and the constant region sequences are derived from a human antibody.

“Antibody-drug conjugate” and “immunoconjugate” refer to an antibody molecule, or antigen-binding portion thereof, including antibody derivatives that binds to CD47, which is conjugated to cytotoxic, cytostatic and/or therapeutic agents.

Antibody molecules of the invention, or antigen-binding portion thereof, can be produced using techniques well known in the art, for example recombinant technologies, phage display technologies, synthetic technologies or combinations of such technologies or other technologies readily known in the art.

The term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

The term “epitope” refers to that portion of a molecule capable of being recognized by and bound by an antibody molecule, or antigen-binding portion thereof, at one or more of the antibody molecule's antigen-binding regions. Epitopes can consist of defined regions of primary secondary or tertiary protein structure and includes combinations of secondary structural units or structural domains of the target recognised by the antigen binding regions of the antibody, or antigen-binding portion thereof. Epitopes can likewise consist of a defined chemically active surface grouping of molecules such as amino acids or sugar side chains and have specific three-dimensional structural characteristics as well as specific charge characteristics. The term “antigenic epitope” as used herein, is defined as a portion of a polypeptide to which an antibody molecule can specifically bind as determined by any method well known in the art, for example, by conventional immunoassays, antibody competitive binding assays or by x-ray crystallography or related structural determination methods (for example NMR).

The term “binding affinity” or “K_{D}” refers to the dissociation rate of a particular antigen-antibody interaction. The K_{D} is the ratio of the rate of dissociation, also called the “off-rate (K_{off})”, to the association rate, or “on-rate (kₒₙ)”. Thus, K_{D} equals K_{off}/Kₒₙ and is expressed as a molar concentration (M). It follows that the smaller the K_{D}, the stronger the affinity of binding. Therefore, a K_{D} of 1 μM indicates weak binding affinity compared to a K_{D} of 1 nM. K_{D} values for antibodies can be determined using methods well established in the art. One method for determining the K_{D} of an antibody is by using surface plasmon resonance (SPR), typically using a biosensor system such as a biosensor system sold under the trademark Biacore®.

The term “potency” is a measurement of biological activity and may be designated as IC₅₀, or effective concentration of an antibody or antibody drug conjugate to the antigen CD47 to inhibit 50% of activity measured in a CD47 activity assay as described herein.

The phrase “effective amount” or “therapeutically effective amount” as used herein refers to an amount necessary (at dosages and for periods of time and for the means of administration) to achieve the desired therapeutic result. An effective amount is at least the minimal amount, but less than a toxic amount, of an active agent which is necessary to impart therapeutic benefit to a subject.

The term “inhibit” or “neutralize” as used herein with respect to bioactivity of an antibody molecule of the invention means the ability of the antibody to substantially antagonize, prohibit, prevent, restrain, slow, disrupt, eliminate, stop, reduce or reverse for example progression or severity of that which is being inhibited including, but not limited to, a biological activity or binding interaction of the antibody molecule to CD47.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “treating”, as used herein, unless otherwise indicated, means reversing, alleviating, inhibiting the progress of, delaying the progression of, delaying the onset of, or preventing the disorder or condition to which such term applies, or one or more symptoms of such disorder or condition. The term “treatment”, as used herein, unless otherwise indicated, refers to the act of treating as defined above. The term “treating” also includes adjuvant and neoadjuvant treatment of a subject. For the avoidance of doubt, reference herein to “treatment” includes reference to curative, palliative and prophylactic treatment. For the avoidance of doubt, references herein to “treatment” also include references to curative, palliative and prophylactic treatment.

It is understood that wherever embodiments are described herein with the language “comprising,” otherwise analogous embodiments described in terms of “consisting of” and/or “consisting essentially of” are also provided.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Unless otherwise defined, all technical and scientific terms used herein have the same meaning as commonly understood by one of ordinary skill in the art to which this invention belongs. In case of conflict, the present specification, including definitions, will control.

Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular. Any example(s) following the term “e.g.” or “for example” is not meant to be exhaustive or limiting.

The practice of the present invention will employ, unless otherwise indicated, conventional techniques of molecular biology (including recombinant techniques), microbiology, cell biology, biochemistry and immunology, which are within the skill of the art.

Particular non-limiting embodiments of the present invention will now be described with reference to accompanying drawings.

### Example 1. Generation of Optimized Anti-CD47 Therapeutic Antibodies

### Introduction

In this example, we successfully generate a panel of antagonistic, optimized anti-CD47 antibodies. These anti-CD47 antibodies are well expressed, biophysically stable, highly soluble and of maximized amino acid sequence identity to preferred human germlines.

### Materials and Methods

### IgG Cloning, Transient Expression, Purification

Antibody v-domain encoding DNA sequences were cloned via restriction-ligation cloning into separate IgG heavy and light-chain expression cassettes in separate plasmid vectors. Antibodies were expressed in two human IgG1 formats: IgG4 (S228P) and IgG1null-IgG1 with the lower hinge mutations L234A/L235A/G237A, which minimise Fcγ receptor-driven effector functions. IgGs were expressed in HEK-293expi or CHO cells after transient transfection with endotoxin-free IgG expression plasmid preparations, per manufacturer's protocols. IgGs were purified using a single-step protocol: Conditioned media were loaded (neat) onto a 1 ml ProA sepharose column, pre-equilibrated in PBS pH7.4. The column was washed with 5 column volumes of PBS pH7.4, before the protein was eluted with 100 mM glycine, pH 2.7 and subjected to dialysis in PBS pH 7.4 using 30 kDa cutoff dialysis membrane.

### IgG Titration Binding ELISAs

To coat ELISA plates sold under the name Greiner Bio-One High Binding ELISA plates, target proteins were diluted to 1 μg/ml in carbonate buffer and added at 100 μl per well, at 4° C., o/n. Coated plates were washed 3× with PBS pH7.4, blocked with 1% BSA in PBS (380 μl/well) for 1 hr at RT, then washed 3× with PBS-non-ionic surfactant sold under the trademark Tween® 20 (PBST). CD47 antibodies (100 μl/well; diluted in PBST) were then added and then incubated 1 hr at RT. Plates were then washed 3× with PBST and goat anti-human kappa chain-HRP added (100 μl/well) at RT, for 1 hr. Plates were then washed 3× with PBST and twice with PBS before the addition of 100 μl TMB per well. Reactions were stopped by adding 100 μl 2M H₂SO₄/well and OD was read on a plate reader at 450 nm.

Anti-CD47 antibodies were tested for polyreactivity by ELISA. Purified, recombinant, target and non-target antigens were coated in 96-well Nunc maxisorp plates at 100 ng per well in carbonate buffer, at 4° C. overnight. Plates were then washed 3× with PBS, blocked with 1% BSA in PBS, then washed 3× with PBS-non-ionic surfactant sold under the trademark Tween® 20. A dilution series of primary antibodies was then applied, plates were washed 3× with PBS-Tween20 followed by application of goat anti-human kappa chain-HRP 1:4,000 secondary antibody. Wells were then washed 3× with PBS-non-ionic surfactant sold under the trademark Tween® 20 and 2× with PBS, 100 μl TMB peroxidase substrate was added per well, the reaction was stopped by adding 100 μl 2M H₂SO₄ and absorbances were read at 450 nm. IgG binding analysis via ELISA on negatively charged biomolecular surfaces were performed as previously described (see Mouquet et al., 2010, Nature 467:591-595).

### CD47 Library Generation and Selection

The CD47 scFv repertoire was assembled by mass oligonucleotide synthesis and PCR. The amplified scFv repertoire was then cloned via restriction-ligation into a phagemid vector, transformed into E. coli TG-1 cells, and the phage repertoire rescued essentially as previously described in detail (Finlay et al., 2011, Methods Mol Biol 681:383-401). Phage selections were performed by coating streptavidin magnetic microbeads with CD47-Fc protein (either human or cyno), washing the beads thrice with PBS and resuspending in PBS pH7.4 plus 5% skim milk protein (MPBS). These beads were coated at 200 nM target protein in round 1 of selection, followed by 100, 50 and 10 nM in subsequent rounds.

### Assay Technology Known Under the Trademark HTRF® Binding Competition Assay

A competition assay technology known under the trademark HTRF® was established to examine epitope competition against h5F9G4 IgG binding to human and/or cyno CD47-Fc by grafted and library-derived clones. The purified h5F9G4 IgG1 was labelled with terbium using a labelling kit sold under the name CisBio per the manufacturer's instructions. The final reaction mix contained biotinylated human or cyno CD47-Fc, SA-XL665 sold under the name CisBio, terbium-labelled parental h5F9G4 and competitor scFv or IgG of interest, prepared as described above, in a total reaction volume of 20 μl in 1× assay buffer [50 mM sodium phosphate, pH 7.5, 400 mM potassium fluoride, and 0.1% BSA (w/v)]. Reagents were added sequentially into 384-well low-volume black plates sold under the name Nunc. Reactions proceeded for 1 h at room temperature, and plates were subsequently read on a plate reader with excitation at 340 nm and two emission readings at 615 nm (measuring input donor fluorescence from h5F9G4-terbium) and 665 nm (measuring output acceptor fluorescence from SAXL665). Readings were expressed as 665 nm/615 nm ratios.

### CD47-SIRPα Binding Competition Assay

A competition ELISA assay was established to examine the capacity of optimized leads to block the binding interaction of CD47 with SIRPxx. To coat ELISA plates sold under the name Greiner Bio-One High Binding ELISA plates, 10 μg/ml human SIRPxx-Fc in carbonate coating buffer was added at 100 μl per well, at 4° C., o/n. Coated plates were washed 3x with PBS pH7.4, blocked with 1% BSA in PBS (380 μl/well) for 1 hr at RT, then washed 3x with PBS-non-ionic surfactant sold under the trademark Tween® 20 (PBST). Biotinylated human, mouse or cyno CD47-Fc was then added at 0.2 g/ml in PBS, 100 μl per well, at room temperature for 60 minutes with or without the addition of competing scFv or IgGs. Plates were then washed 3× with PBST and Streptavidin-HRP added (100 μl/well) at room temperature, for 1 hr. Plates were then washed 3× with PBST and twice with PBS before the addition of 100 μl TMB per well. Reactions were stopped by adding 100 μl 2M H₂SO₄/well and OD was read on a plate reader at 450 nm.

Antibody v-Domain T Cell Epitope Content: In Silico Analyses

In silico technologies by the company known as Abzena, Ltd., which are based on identifying the location of T cell epitopes in therapeutic antibodies and proteins, were used for assessing potential immunogenicity in antibody v-domains. Immunogenicity assessment tool known under the trademark iTope™ was used to analyse the VL and VH sequences of key leads for peptides with promiscuous high affinity binding to human MHC class II. Promiscuous high affinity MHC class II binding peptides are thought to correlate with the presence of T cell epitopes that are high risk indicators for clinical immunogenicity of drug proteins. The immunogenicity assessment tool known under the trademark iTope™ software predicts favourable interactions between amino acid side chains of a peptide and specific binding pockets (in particular pocket positions; p1, p4, p6, p7 and p9) within the open-ended binding grooves of 34 human MHC class II alleles. These alleles represent the most common HLA-DR alleles found world-wide with no weighting attributed to those found most prevalently in any particular ethnic population. Twenty of the alleles contain the ‘open’ p1 configuration and 14 contain the ‘closed’ configuration where glycine at position 83 is replaced by a valine. The location of key binding residues is achieved by the in silico generation of 9mer peptides that overlap by eight amino acids spanning the test protein sequence. This process successfully discriminates with high accuracy between peptides that either bind or do not bind MHC class II molecules.

In addition, the sequences were analysed using a database known under the trademark TCED™ (T Cell Epitope Database™) search for matches to T cell epitopes previously identified by in vitro human T cell epitope mapping analyses of other protein sequences. The database known under the trademark TCED™ is used to search any test sequence against a large (>10,000 peptides) database of peptides derived from unrelated protein and antibody sequences.

### Phagocytosis Potency Assays Using Human Macrophages

Peripheral blood mononuclear cells (PBMCs) were isolated from whole blood by density gradient centrifugation. CD14 positive PBMCs were subsequently isolated via magnetic cell isolation using CD14 microbeads. In parallel, Jurkat cells were labelled using a green CFSE (carboxy-fluorescein diacetate, succinimidyl ester) cell tracer dye. A total of 6.25×10⁵ labelled Jurkat cells were pre incubated in the presence of UH2 antibodies in 96 well plates for 1 hour at 37 degrees C. in a humidified atmosphere containing 5% CO2. Following incubation, 2.5×10⁵ CD14 positive cells were added to each well and incubated for a further hour under the same culture conditions.

Cells were harvested by vigorous pipetting, stained with viability dye, fixed using ice-cold 4% paraformaldehyde for 10 minutes. Following fixation, cells were blocked with an Fc receptor binding inhibitor monoclonal antibody for 10 minutes and then incubated with a red fluorescent dye sold under the trademark Alexa Fluor® 647 (AF647) conjugated anti-human CD11b antibody at room temperature for 30 minutes and fixed a further time in 4% paraformaldehyde for 5 minutes.

Cells were analysed on a flow cytometer sold under the trademark BD LSRFortessa™ recording side scatter and forward scatter properties along with CFSE and AF647 fluorescence intensity data. Data was captured until at least 1×10⁴ AF647 positive events were recorded. Data were analysed post-acquisition using software known under the trademark FlowJo™ (version 10.4.2). Briefly, cell debris was gated out by scatter properties (SSC-Area by FSC-Area). Single cells were also gated for by SSC-Area by SSC-Height and then by FSC-Area by FSC-Height. From the remaining single cell population, CFSE and CD11b double positives cells were gated using a quadrant gate placed based on the population of CD11b positive cells in the vehicle treated test. The percentage of CFSE positive cells from the CD11b positive population was calculated and plotted.

### Results and Discussion

CDR Grafting onto Preferred Human Germline v-Genes

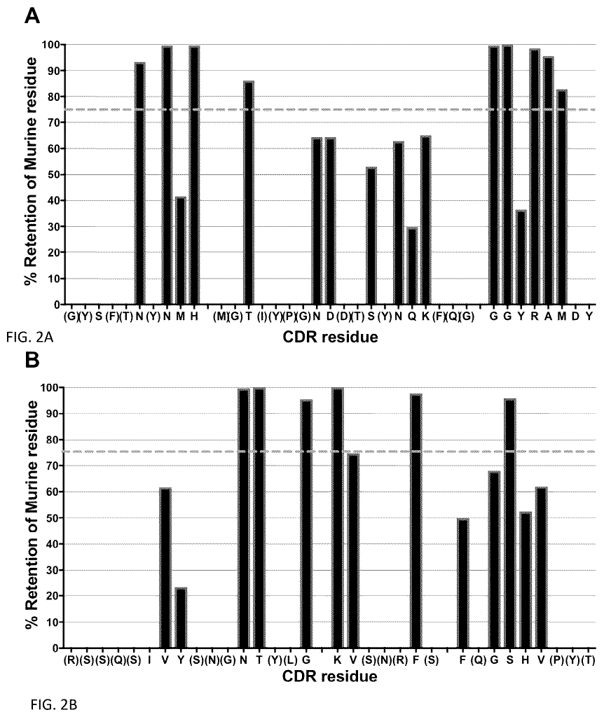

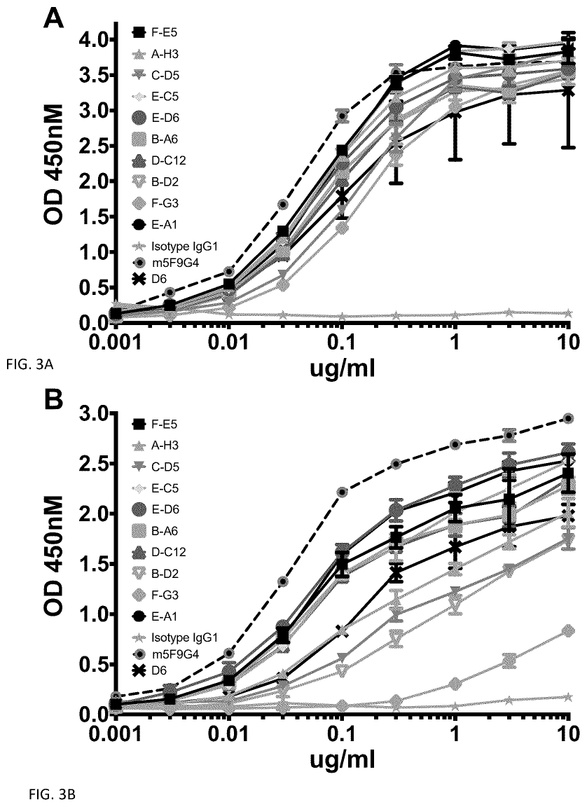

The CDRs of an antagonistic murine anti-CD47 IgG 5F9G4 (5F9G4; see WO2011143624A2 and Table 2) were initially introduced to human germline immunoglobulin v-domain framework sequence scaffolds using CDR grafting. To bias our engineering efforts towards final lead therapeutic IgG compounds with optimal drug-like properties, we chose to graft the CDRs of the parental antibody onto “preferred” germline scaffolds IGHV5-51 and IGKV2-28, which are known to be very well displayed and expressed in scFv format in phage display, have good solubility, high physical stability and are used at high frequency in the expressed human antibody repertoire. IGHV5-51 was also the preferred germline framework for grafting of the VH as it showed high sequence and structural motif homology with the m5F9G4 HCDR2 (FIG. 2).

Those scaffolds and grafted CDR definitions are outlined in Table 2. The heavy and light chain sequences for chimeric anti-CD47 antibody m5F9G4 and humanized h5F9G4 are also shown in Table 2. While this process of CDR grafting is well known, it is still problematic to predict whether a given set of human v-domain sequences will act as suitable acceptor frameworks for non-human CDR grafting. The use of unsuitable frameworks can lead to the loss of target binding function, protein stability issues or even impaired expression of the final IgG. The IGKV2-28/IGHV5-51 graft was therefore taken forward as the template for CDR mutagenesis and selection of improved clones.

Library Generation and Screening The CDR-grafted IGKV2-28/IGHV5-51 v-domain sequences were combined into a VL-VH scFv format and a mutagenesis library cassette was generated by mass oligonucleotide synthesis and assembly. The final scFv library was ligated into a phage display vector and transformed into E. coli via electroporation to generate 6.0×10⁸ independent clones. Library build quality was verified by sequencing 96 clones. This sequencing data showed that the positions encoding either the murine or human germline residue at each position of variance had been effectively sampled at a frequency of approximately 50%. Libraries were rescued using helper phage M13 and selections performed on biotinylated human and cynomolgus monkey CD47-Fc proteins in multiple separate branches.

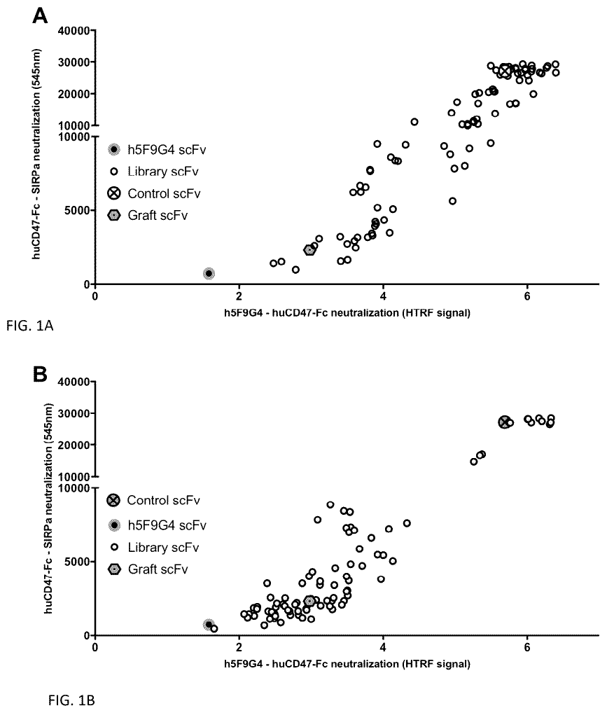

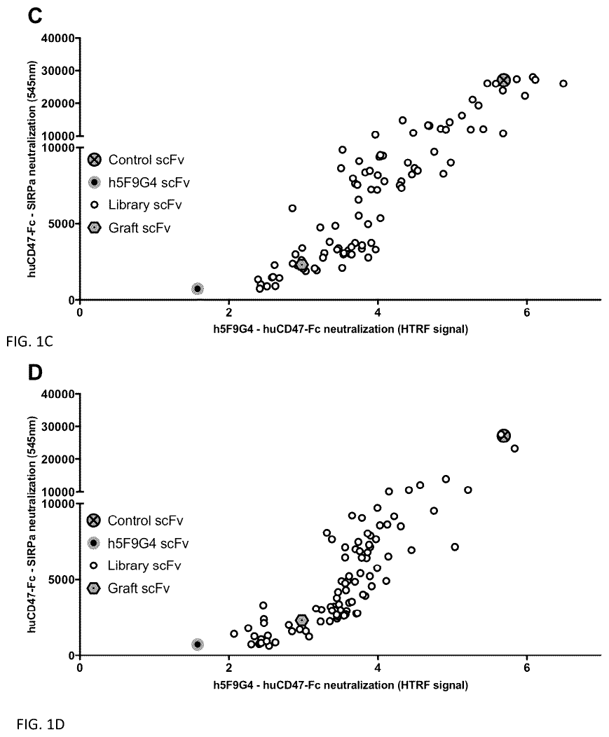

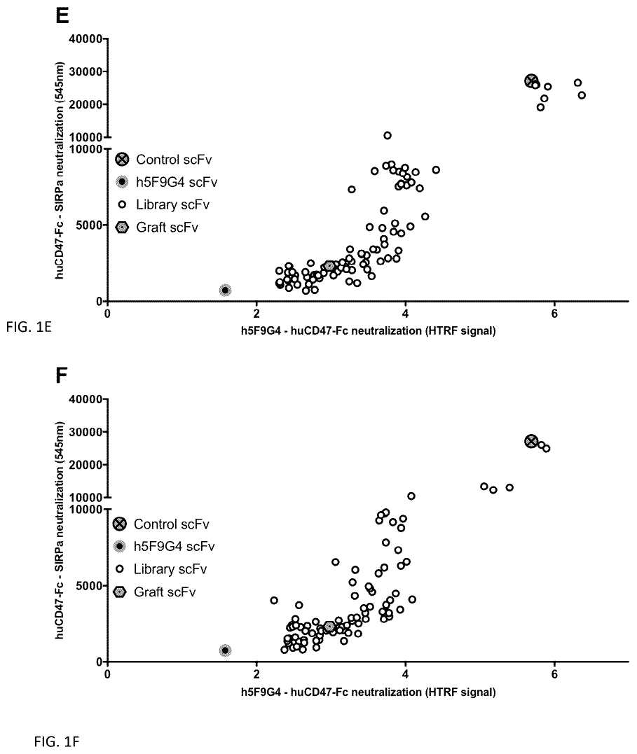

Post-selection screening (as shown in FIG. 1A-F) and DNA sequencing revealed the presence of 269 unique, human and mouse CD47-binding scFv clones that retained epitope binding competition with h5F9G4 IgG1, blocked the binding of hCD47 to hSIRPax and contained significantly increased human content within the CDRs, while the framework sequences remained fully germline. Amongst these 269 clones, germ-lining mutations were observed in all CDRs (Table 3). Lead clones were ranked based on the level of CDR germ-lining versus assay technology known under the trademark HTRF® signals for both human CD47-Fc and h5F9G4 (FIG. 1). The v-domains of the 11 top clones from this ranking were then sub-cloned into IgG expression vectors for further testing as below (Table 4). Unexpectedly however, in all analyses of both CD47-SIRPαand CD47-h5F9G4 neutralisation, none of the library derived clones, nor the IGKV2-28/IGHV5-51 graft scFv achieved the same potency as the IGKV2-28/IGHV1-3-grafted h5F9G4 scFv (FIG. 1A-F).

While germ-lining mutations were observed in all CDRs for the lead clones derived directly from library selections, it remained possible that sequence analyses might allow further clones to be designed to have maximal humanization and retain maximal CD47 binding affinity and antagonism potency. The 269 sequence-unique hits with binding signals against human and mouse protein were therefore used to analyse the retention frequency for murine amino acids in the CDRs of this functionally characterized population. Positional amino acid retention frequency was expressed as a percentage found in the VH and VL domains (FIG. 2A, 2B). Murine residues with RF<75% were regarded as positions that are possibly not essential to the target-binding paratope and are likely to be open to germ-lining, in a series of combinatorial designs. Importantly, as observed in the scFv screening data outlined above, the potency of the IGHV5-51-grafted VH domains was deemed likely to be lower than those on a IGHV1-3 framework. To examine whether or not this was the case, the paratope sampling information outlined in FIG. 2A and B was used to construct designer clones in the IGKV2-28 and IGHV1-3 frameworks for comparison alongside the heavily-humanized clones derived from the initial library (IGHV5-51 framework in the VH domain).

Ten VH designs containing principally those murine residues with RF >75%, in a number of combinations, were designated VH1.1-VH1.10. Six VL designs were also created (VL1.1-VL1.6) that combined the most humanized CDRs observed in the high-functioning population of scFv sequences, plus further-humanizing and deamidation motif-disrupting mutations in the LCDR1 (Table 4). These LCDR1 humanizing and stabilising mutations sampled positions 11, 12 and 13 (residues GNT), which had been observed to be variable positions (FIG. 2A, Table 3), but had not explicitly demonstrated that these potentially optimal sequences (e.g. the ‘ANT’ and especially ‘AYN’ motifs found in designer chain VL1.4 and others outlined in Table 4) could be tolerated while retaining target binding specificity and the ability to block CD47-SIRPα interaction.

The VH and VL clones were generated by gene synthesis (along with the 11 library-derived clones outlined above, positive controls IGHV5-51 graft, h5F9G4 and m5F9G4, and negative control non-CD47-reactive v-domains), then cloned into expression vectors for production as human IgG1null. To maximise the opportunity to find functional optimised sequences, the designer VH and VL clones were matrixed to combine all potential combinations of VH and VL sequences. To examine whether or not the observed reduced potency of the IGHV5-51-based scFvs observed above was due to scFv formatting and not the framework choice, the scFv of lead clone D6 was cloned as both IgG1 and scFv-Fc fusion formats. All IgGs and the D6 scFv-Fc fusion protein were readily expressed and purified from transient transfections of HEK-293 cells.

### Library-Derived Lead IgG Specificity and Potency Characteristics

The purified IgGs described above were then tested for binding to human and cyno CD47-Fc in direct titration ELISA format. This analysis demonstrated that while all library derived clones clones retained binding affinity for human CD47, none fully recapitulated the binding of m5F9G4 IgG1 (FIG. 3A). This reduced binding was more pronounced on cyno CD47, with clone F-G3 showing particularly weak reactivity (FIG. 3B).

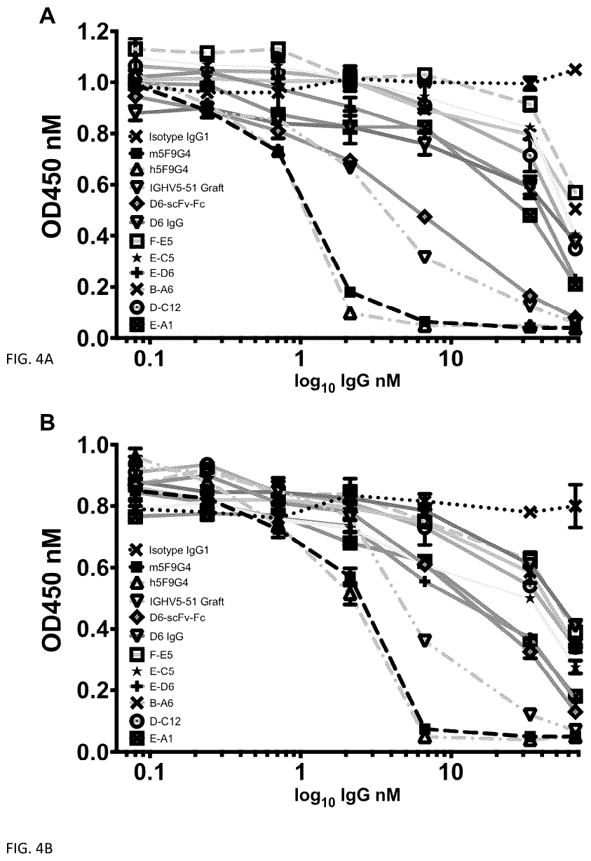

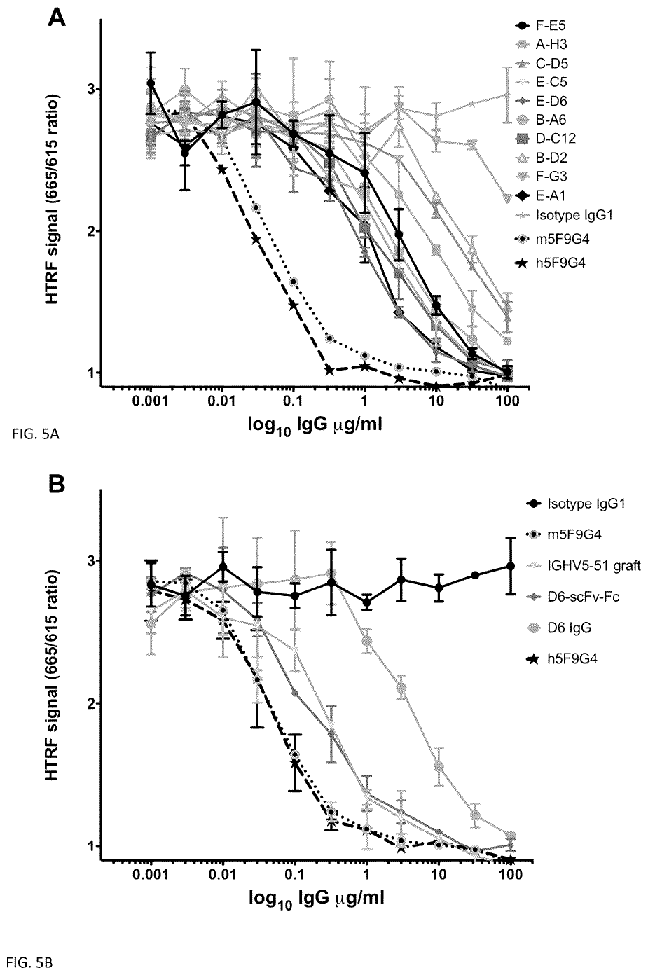

As direct ELISA binding signal is influenced by avidity and does not prove the maintenance a specific epitope, all IgGs were then examined in a CD47-SIRPαbinding blockade assay (FIG. 4) and a solution-phase assay technology known under the trademark HTRF® competition assay of 5F9G4 binding to CD47 (FIG. 5). In the CD47-SIRPax binding blockade assay, all library-derived IgGs exhibited significantly reduced potency in comparison to h5F9G4, m5F9G4 and the IGHV5-51 grafted IgG, whether binding to human (FIG. 4A) or cyno CD47 (FIG. 4B). Importantly, the IGHV5-51-grafted IgG demonstrated the ability to fully block the binding of both human and cyno CD47 to SIRPα, but with demonstrably reduced potency in comparison to h5F9G4. This suggested that the grafting process of placing the F9G4 CDRs onto the IGHV5-51 framework had specifically reduced the potency of the molecule in comparison to h5F9G4, as the CDR sequences are maintained across both frameworks. In a further unexpected finding, the D6 scFv-Fc fusion protein maintained the starting CD47 blocking potency of the IGHV5-51 graft IgG against both orthologs, while the D-A6 IgG did not and was incapable of fully blocking either ortholog (FIG. 4A, 4B).

To ensure that the observed loss of CD47 blockade potency by some IGHV5-51-framework clones was not driven by the phenomenon of ‘epitope shift’, an assay technology known under the trademark HTRF® assay was performed. This assay examined the ability of these clones to compete for the binding of h5F9G4 to CD47. These analyses showed that almost all clones (F-G3 excepted) could cross-compete with h5F9G4, with most clones demonstrating full inhibition of binding at the highest concentration (FIG. 5A). Potency of inhibition was again reduced in comparison to h5F9G4 self-competition, however. This finding suggested maintenance of a shared epitope, but reduced binding affinity in the IGHV5-51 framework clones. Importantly, the improved function of the D6 scFv-Fc over the D6 IgG was again demonstrated, with the D6 scFv-Fc showing equivalent potency to the IGHV5-51 graft IgG, while the D-A6 IgG was clearly reduced potency (FIG. 5B).

The findings regarding D6 scFv-Fc fusion potency versus D6 IgG strongly suggested that the process of re-grafting the CDRs, coupled with scFv display and screening had unexpectedly selected for clones which are preferentially functional in scFv format, which are then impaired when reconfigured into IgG1. This observation renders such clones particularly attractive in bispecific antibody generation as scFv fragments can be successfully used as modular building blocks of multi-specific antibodies. In totality, however, these findings also illustrate that the original methods of Townsend et al. had failed to generate an improved version of h5F9G4

### Designer IgG Specificity and Potency Characteristics

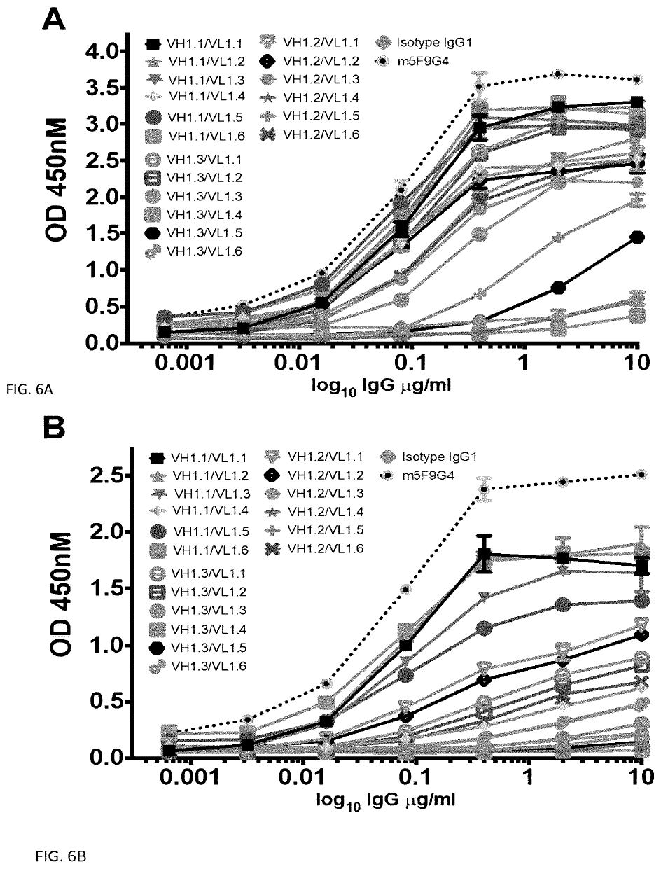

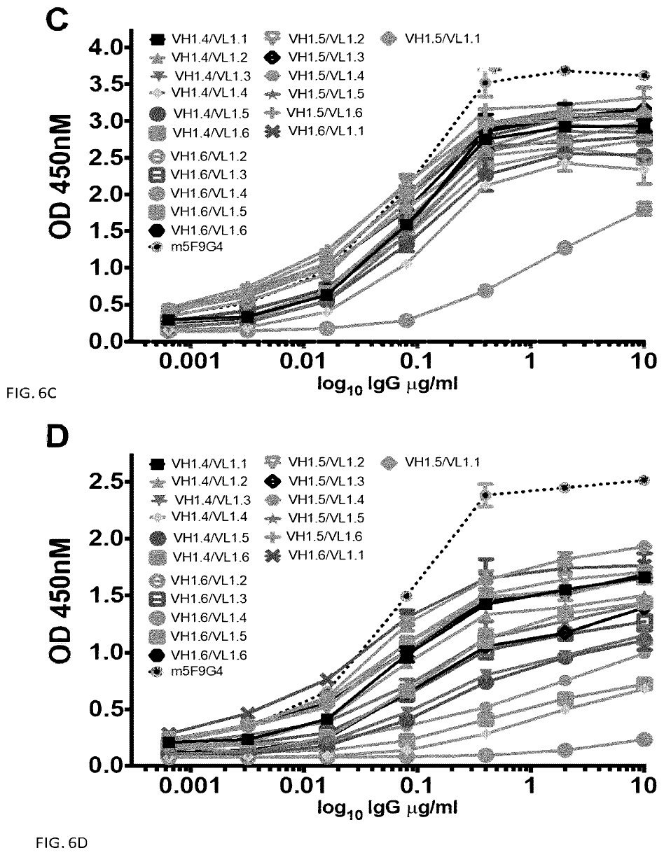

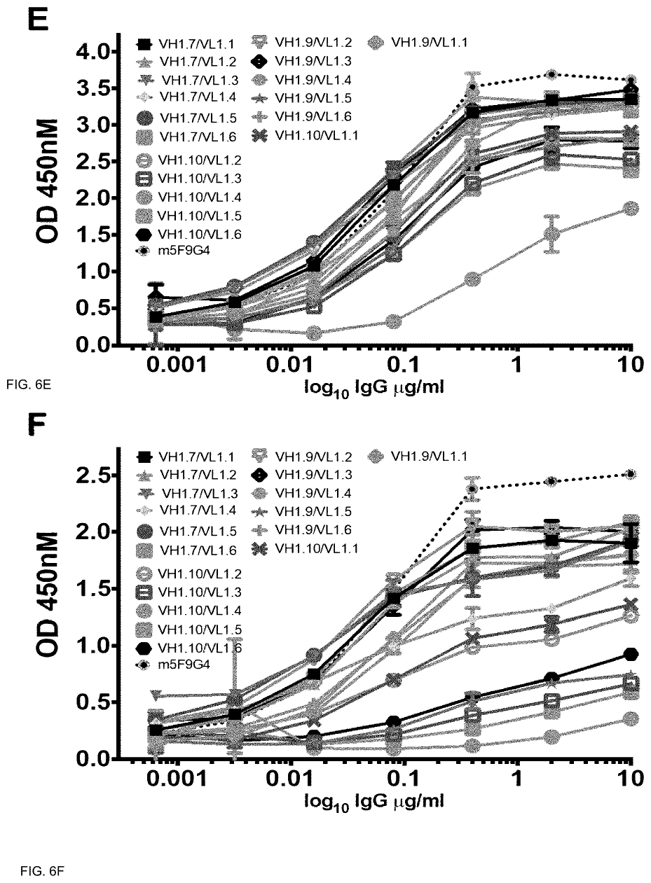

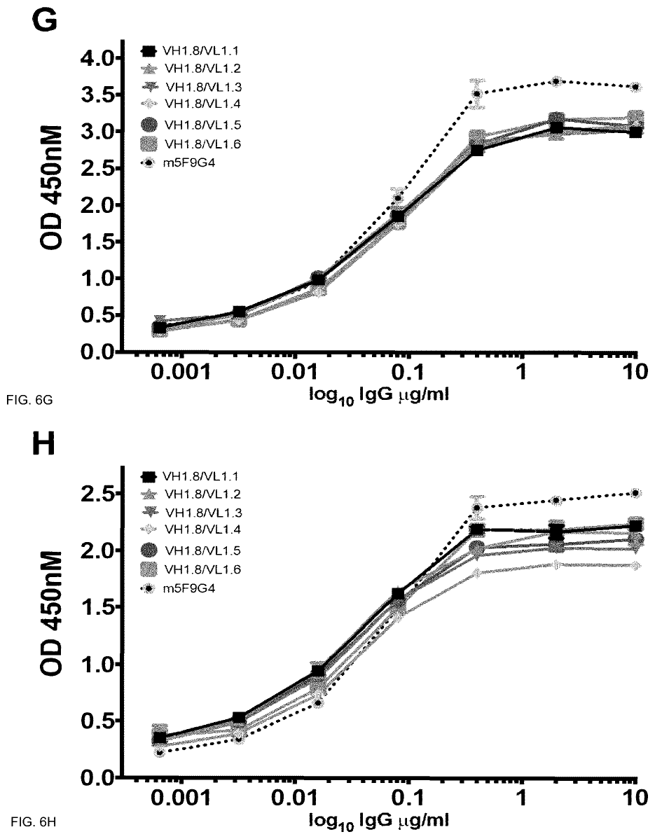

The purified designer IgGs described above were then tested for binding to human and cyno CD47-Fc in direct titration ELISA format. This analysis demonstrated that while many clones retained binding affinity for human and cyno CD47, the range of binding potencies was highly heterogeneous, with some clones being significantly reduced in binding signal, but with a subset of IgGs fully recapitulating the binding of m5F9G4 IgG1 (FIG. 6A-H). The family of clones containing the VH1.8 VH domain in combination with the VL1.1-1.6 domains demonstrated the most consistent recapitulation of the full binding potency of m59G4 and are shown separately in FIG. 6G and H.

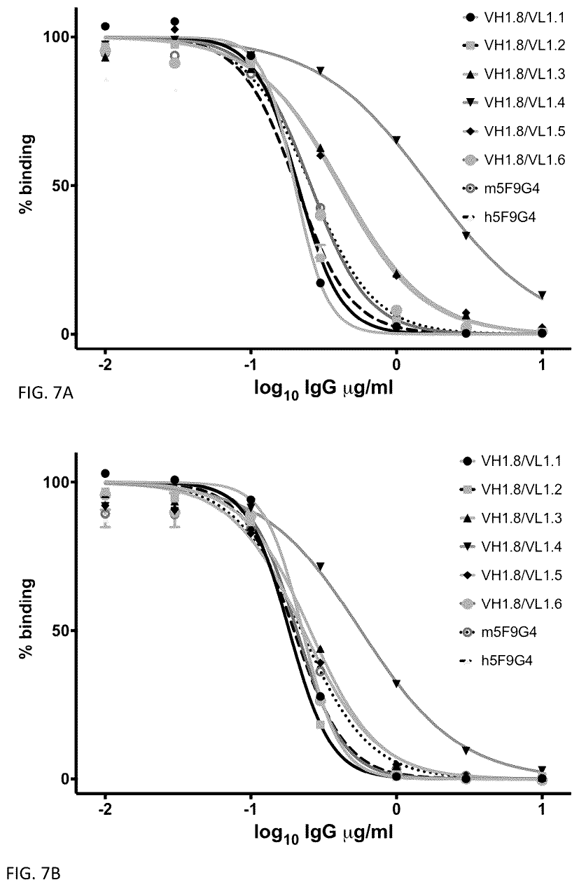

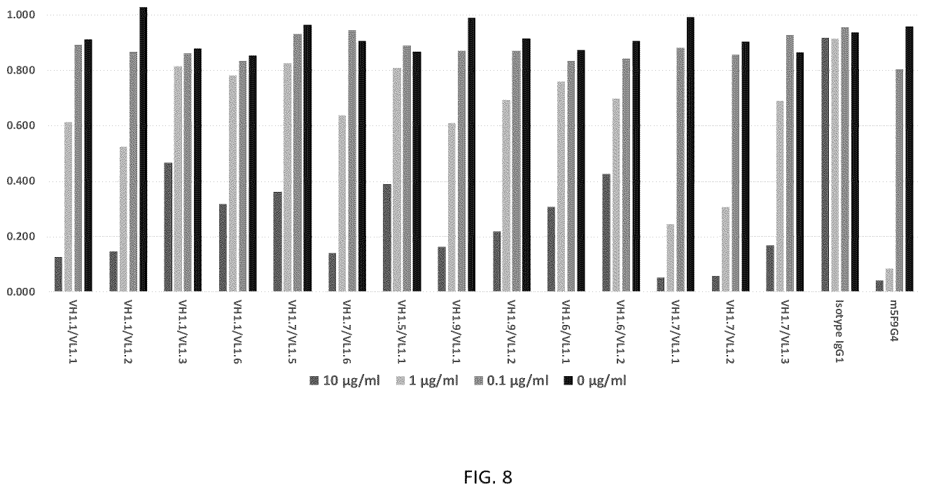

As direct ELISA binding signal is influenced by avidity and does not prove the maintenance a specific epitope, all IgGs were then examined in a CD47-SIRPαbinding blockade assay (FIG. 7, FIG. 8). In the CD47-SIRPαbinding blockade assay, all VH1.8 family IgGs exhibited high potency, with functional equivalency for all clones other than VH1.8/VL1.4, in comparison to h5F9G4 and m5F9G4, whether binding to human (FIG. 7A) or cyno CD47 (FIG. 7B). Several other designer clones which had performed well in ELISA also exhibited the ability to block hCD47-SIRPαbinding (FIG. 8), albeit with lower potency than the VH1.8 family clones.

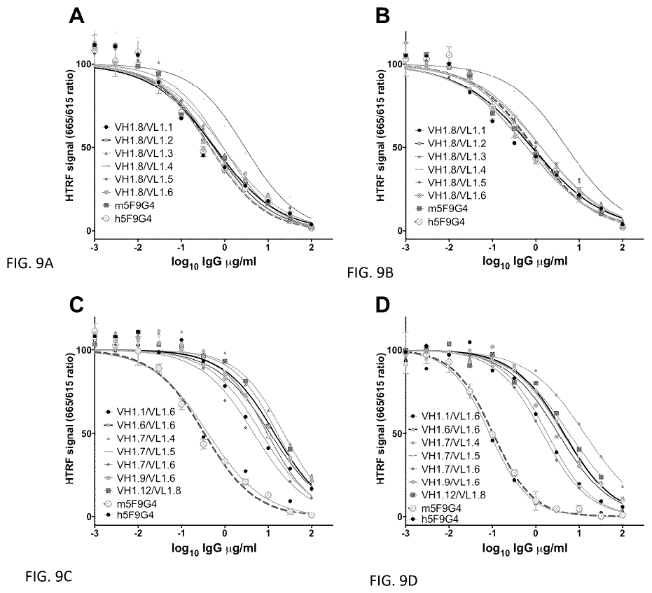

To ensure that the observed maintenance of CD47 blockade potency by high-functioning designer clones was coupled with maintained epitope specificity, a solution-phase assay technology known under the trademark HTRF® competition assay of 5F9G4 binding to CD47 was performed (FIG. 9). This assay examined the ability of the key designer clones to compete for the binding of h5F9G4 to CD47. These analyses showed that all VH1.8 family clones could cross-compete with h5F9G4, with multiple clones demonstrating equivalent (Table 5) potency against human (FIG. 9A) and cyno (FIG. 9B) CD47. The potency of inhibition for VH1.8/VL1.4, however, was again reduced in comparison to h5F9G4 self-competition. This finding suggested maintenance of a shared epitope, but reduced binding affinity for the VL1.4 domain-containing clones. Further assay technology known under the trademark HTRF® analyses showed that all non-VH1.8 family clones which demonstrated hCD47 blockade (FIG. 8) could also cross-compete with h5F9G4, but with all clones demonstrating reduced potency (Table 5) against human (FIG. 9C) and cyno (FIG. 9D) CD47. In an attempt to further sample CDR residues in the VH1.8 background, two further designs (VH1.11 and VH1.12) were generated, along with 3 more designer descendants of the VL1.5 sequence (VL1.7, VL1.8 and VL1.9). These designs were co-expressed in a matrix to produce 6 further IgGs, then examined in the CD47-SIRPαblockade assay. This analysis showed that all of the clones had lost all potency in antagonising CD47, other than VH1.12/VL1.8, which maintained minimal blocking (<50%) at 10 μg/ml. These findings showed that the mutation of F>A at position 6 of the LCDR2, or the mutation F>M at position 1 of the LCDR3 both led to the full ablation of CD47 antagonism.

### Flow Cytometric Analyses of Lead IgG Binding Specificity at the Cell Membrane

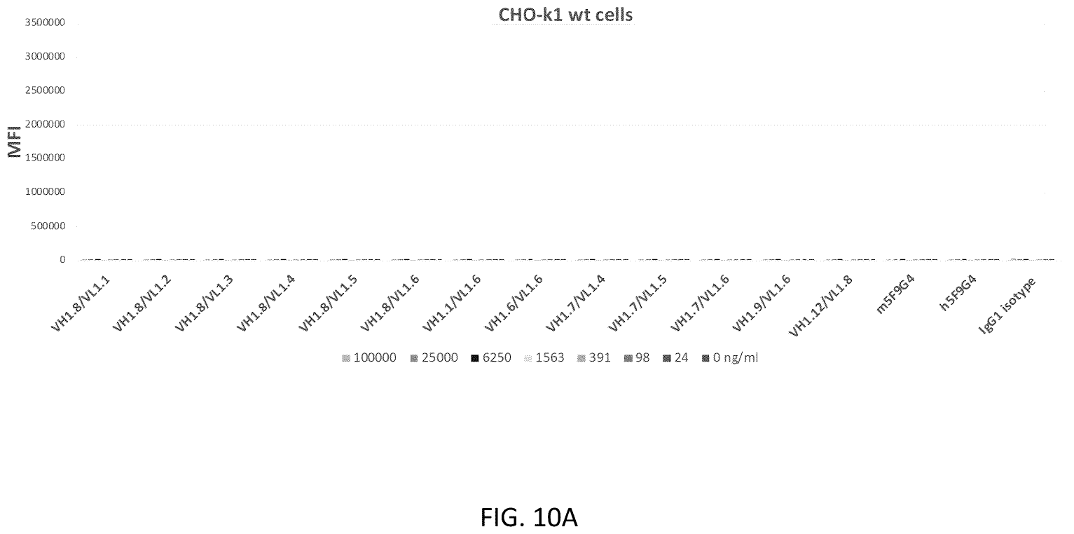

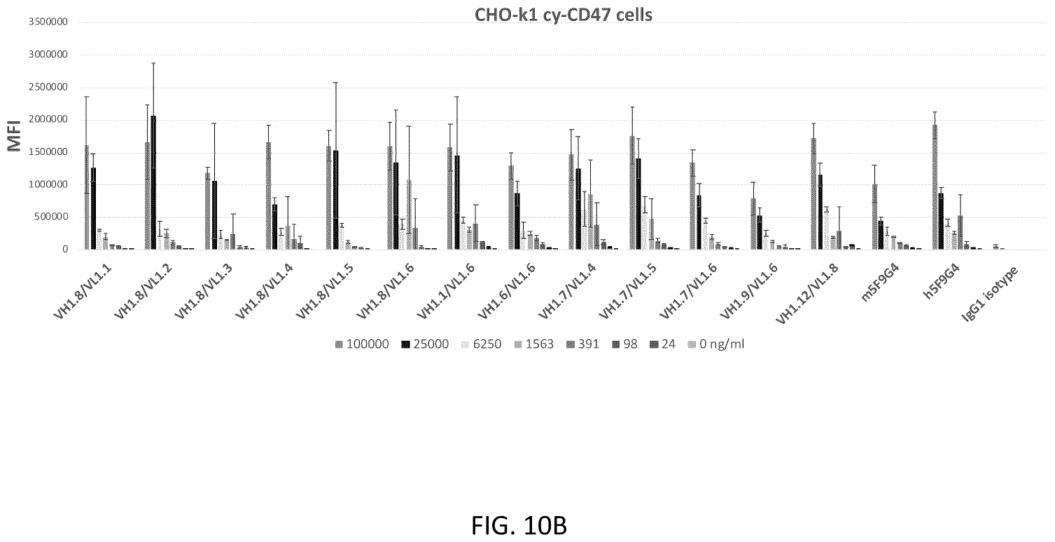

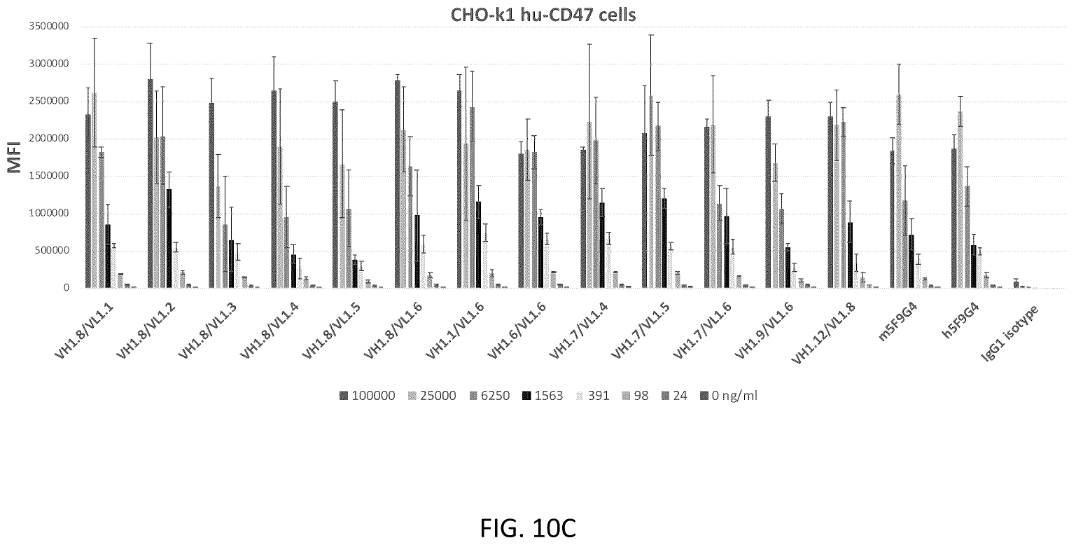

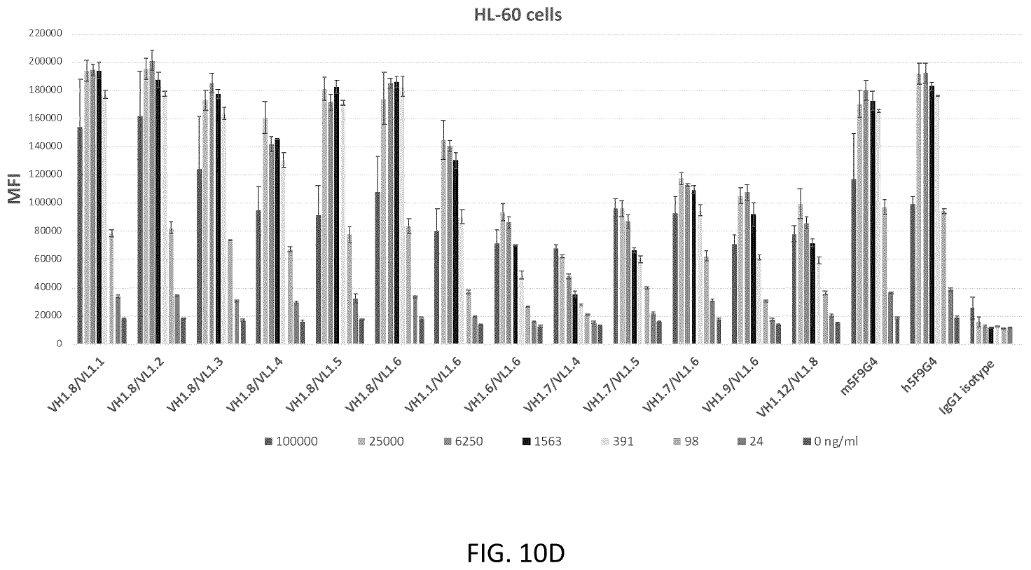

Antibodies shown to retain binding to CD47 by ELISA were analysed for concentration-dependent binding at the cell surface via flow cytometry. CHO-K1 cells were stably transfected with either human or cyno CD47 full-length cDNAs. Anti-CD47 IgGs and an isotype control IgG1 were then all tested in IgG1null format, over a concentration range of 100-0.024 μg/ml for binding to human, cyno, or wild type control (‘wt’, i.e. untransfected) CHO-K1 (FIGS. 10A-D). All IgGs other than the isotype control showed concentration-dependent binding to human and cyno CD47+ cells, and also to human AML cell line HL60, with a maximum MFI in each case being >50-fold higher than observed background signals for binding to untransfected CHO-K1. Anti-CD47 antibodies exhibited no measurable background binding on untransfected CHO-K1 cells, comparable to the Isotype control IgG1 (FIGS. 10A-D).

### Lead IgG Analyses in ‘Developability’ ELISA Assays

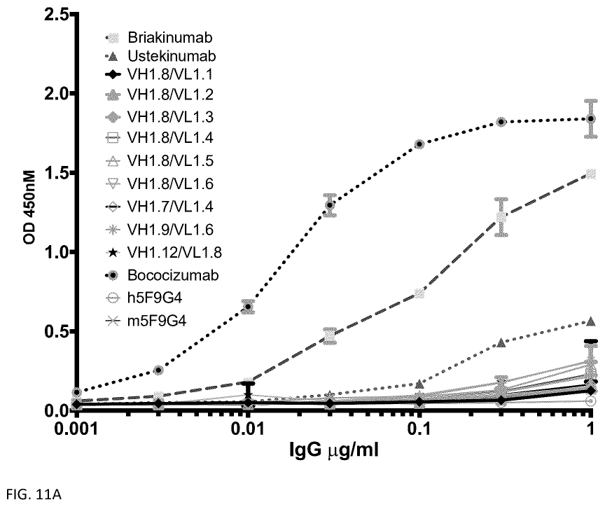

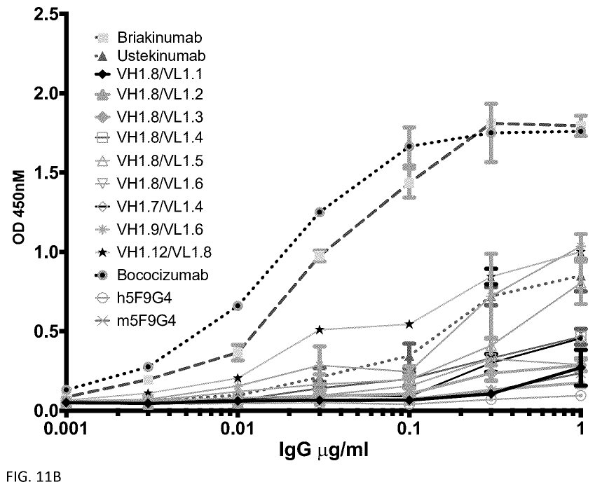

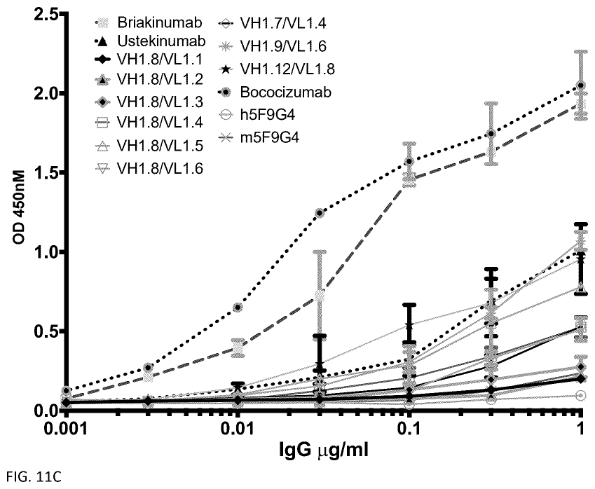

It is known in the art that the binding of IgGs intended for therapeutic use to several indicative biological substrates is an indicator of high risk for poor performance in patients due to poor bioavailability and short in vivo half-life. Three such biological substrates are Insulin, dsDNA and ssDNA (Avery et al. Mabs; 10:2; 244-255; 2018). These three substrates were therefore used to coat ELISA plates and examine the binding of the IgG1null versions of the optimised lead antibodies. Binding signals for these human IgG-based antibodies was compared to ‘positive control’ human IgG antibodies that have been found to have polyreactivity and poor performance, which stopped their progress in clinical trials (Bococizumab and Briakinumab human IgG1 analogues). For a negative control human IgG1 antibody, an IgG1 Ustekinumab analogue was used as it reacts with the same therapeutic target as Briakinumab, but has longer pK and was successfully approved as a therapeutic product. In the ELISA analyses shown in FIG. 11A, B and C, the positive control antibodies exhibited the expected strong reactivity to all 3 substrates, while the negative control Ustekinumab showed low reactivity. Importantly, all of the IgG1 lead clones tested showed binding≤the negative control against all 3 substrates. This finding underlined the maintenance of highly specific, target-driven binding in the optimised clones VH1.8/VL1.1, VH1.8/VL1.2, VH1.8/VL1.3, VH1.8/VL1.4, VH1.8/VL1.5, VH1.8/VL1.6, VH1.7/VL1.4, VH1.6/VL1.9 and VH1.12/VL1.8.

Antibody v-Domain T Cell Epitope Analyses

In silico technologies by the company known as Abzena, Ltd., which are based on identifying the location of T cell epitopes in therapeutic antibodies and proteins, were used for assessing the immunogenicity of both the h5F9G4 and lead antibody v-domains. Analysis of the v-domain sequences was performed with overlapping 9mer peptides (with each overlapping the last peptide by 8 residues) which were tested against each of the 34 MHC class II allotypes. Each 9mer was scored based on the potential ‘fit’ and interactions with the MHC class II molecules. The peptide scores calculated by the software lie between 0 and 1. Peptides that produced a high mean binding score (>0.55 in the immunogenicity assessment tool known under the trademark iTope™ scoring function) were highlighted and, if >50% of the MHC class II binding peptides (i.e. 17 out of 34 alleles) had a high binding affinity (score >0.6), such peptides were defined as ‘high affinity’ MHC class II binding peptides which are considered a high risk for containing CD4+ T cell epitopes. Low affinity MHC class II binding peptides bind a high number of alleles (>50%) with a binding score >0.55 (but without a majority >0.6). Further analysis of the sequences was performed using the database known under the trademark TCED™. The sequences were used to interrogate the database known under the trademark TCED™ by BLAST search in order to identify any high sequence homology between peptides (T cell epitopes) from unrelated proteins/antibodies that stimulated T cell responses in previous in vitro T cell epitope mapping studies performed at the company known as Abzena Ltd.

Peptides were grouped into four classes: High Affinity Foreign (‘HAF’-high immunogenicity risk), Low Affinity Foreign (‘LAF’-lower immunogenicity risk), TCED+ (previously identified epitope in the database known under the trademark TCED™), and Germline Epitope (′GE′-human germline peptide sequence with high MHC Class II binding affinity). Germline Epitope 9mer peptides are unlikely to have immunogenic potential due to T cell tolerance (i.e. these peptides are recognised as ‘self in the host), as validated by previous studies with a wide range of germline peptides. Importantly, such germline v-domain epitopes (aided further by similar sequences in the human antibody constant regions) also compete for MHC Class II occupancy at the membrane of antigen presenting cells, reducing the risk of foreign peptide presentation being sufficient to achieve the ‘activation threshold’ required for T cell stimulation. High GE content is therefore a beneficial quality in clinical development of an antibody therapeutic. On the other end of the scale, TCED+peptides are very high risk for immunogenicity in the clinic, as they have the characteristics of experimentally-proven peptide epitopes that can be presented on MHC molecules and activate human t cells.

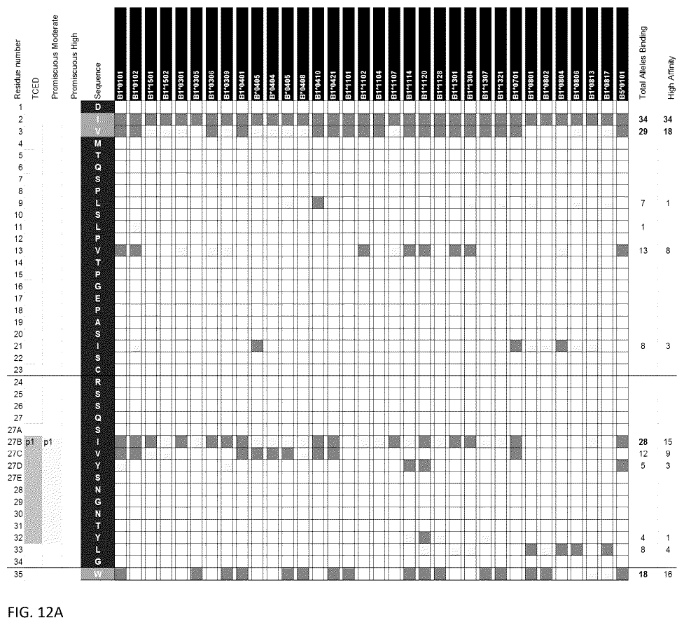

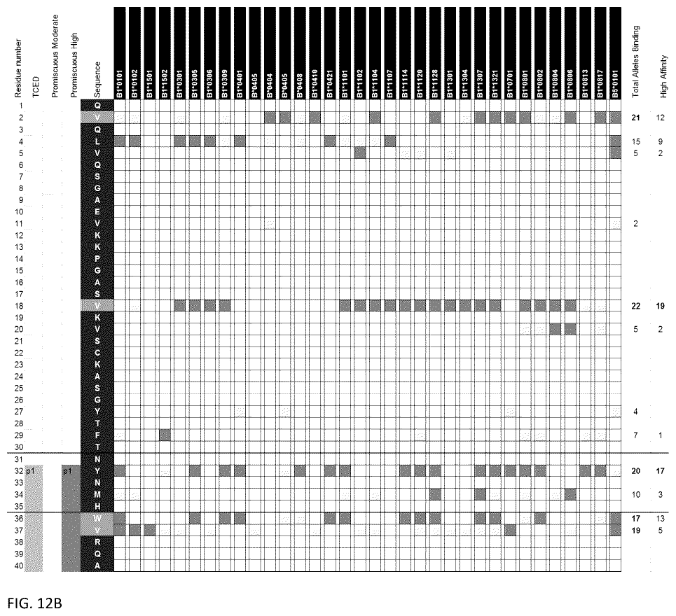

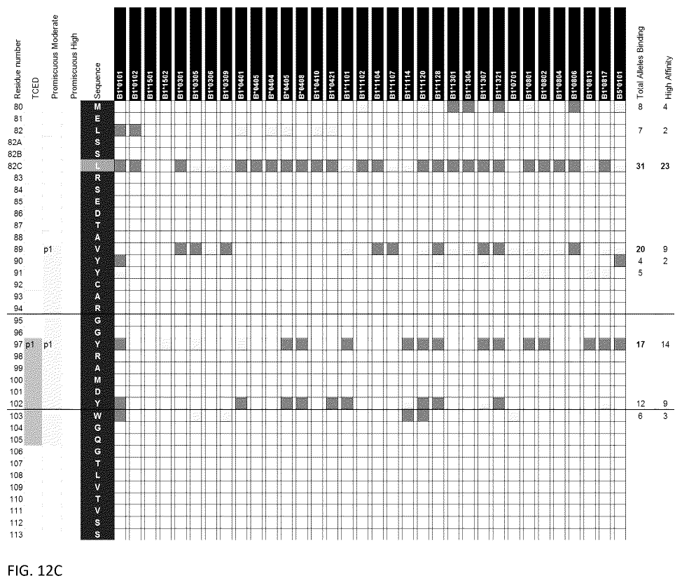

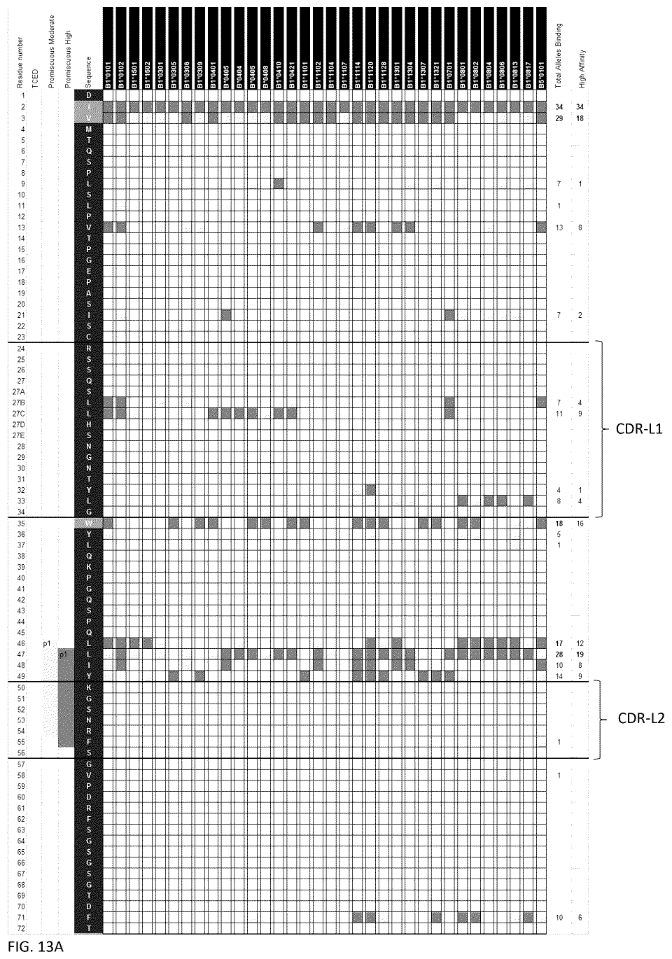

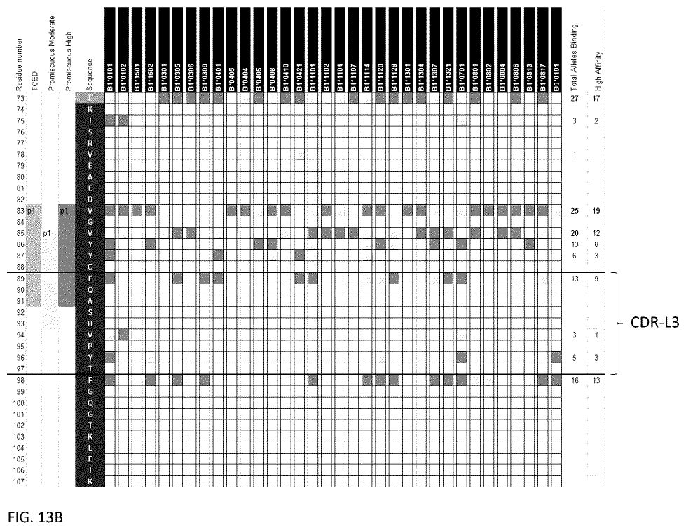

Key lead v-domains exhibited significant beneficial changes in peptide epitope content in comparison to h5F9G4. The v-domain engineering process undertaken here had therefore successfully selected for antibodies that maintained anti-CD47 potency, while removing multiple HAF and LAF epitopes that were TCED+ and found in both the heavy and light chain v-domains of h5F9G4. Importantly, these foreign epitopes were specifically eliminated by germlining mutations found in the CDRs of lead clones. For example, a TCED+ peptide ‘IVYSNGNTY’ (SEQ ID NO: 106) (FIG. 12A) found in the LCDR-1 of h5F9G4 (and, therefore, in any forms of h5F9G4 humanized by CDR grafting) was eliminated in the majority of lead clones by the mutation I>L and V>L at positions 1 and 2, as found in all lead clones in Table 4. This finding illustrated that germlining and non-germlining mutations in the LCDR1 alone are sufficient to remove not only asparagine deamidation risk motifs, but also a proven human T cell epitope.

Similarly, the HCDR1/Framework 2 region from the h5F9G4 sequence encoded for a HAF and TCED+peptide ‘YNMHWVRQA’ (SEQ ID NO:107) (FIG. 12B). This epitope was ablated in a number of lead sequences found in Table 4, such as antibodies containing the sequence ‘YAMHWVRQA’ (SEQ ID NO:108) (e.g. VH1.1, VH1.6, VH1.8, VH1.9, VH1.11, VH1.12), or ‘YNIHWVRQA’ (SEQ ID NO:109) (e.g. VH1.7). Importantly, the mutations N>A at position 2 and M>I at position 3 not only ablated the TCED+epitope, but converted this epitope into GE peptides. In addition, the germlining of the last residues of the HCDR2 of all leads described in Table 4 (mutation D>G at the final c-terminal HCDR2 position as defined in Table 2), also reinstated GE peptides that are found in the germlines of both IGHV5-51 (‘FQGQVTISA’ SEQ ID NO:102) and IGHV1-3 (‘FQGRVTITA’ SEQ ID NO:103).

In the HCDR3/Framework 4 sequence of h5F9G4 VH domain, a further TCED+peptide ‘YRAMDYWGQ’ (SEQ ID NO:110) was identified (FIG. 12C). This epitope was ablated in a number of lead sequences found in Table 4, such as antibodies containing the sequence ‘YRAEDYWGQ’ (SEQ ID NO:111) (e.g. VH1.1, VH1.2, VH1.3, VH1.6, VH1.9).

Critically, non-germline and germline mutations that were predicted not to be tolerated in CD47-binding clones according to the data in FIGS. 2A and 2B were included in several key leads, as exploratory, novel substitutions. For example, in the LCDR-1 of h5F9G4 the development risk motif-removing mutations found in light chain sequences VL1.3, VL 1.4, VL 1.5 and VL 1.6 (Table 4) should not be tolerated (FIG. 2B). Similarly, the single, deimmunising, non-conservative, N>A mutation in the HCDR1 of clones based on the VH1.8 sequence and others (Table 4) was strongly predicted to be not tolerated in the data of FIG. 2A). These findings illustrate that beneficial mutations are contextual, v-gene framework-dependent and in this case could not be fully predicted either a priori, or by the method of Townsend et al.

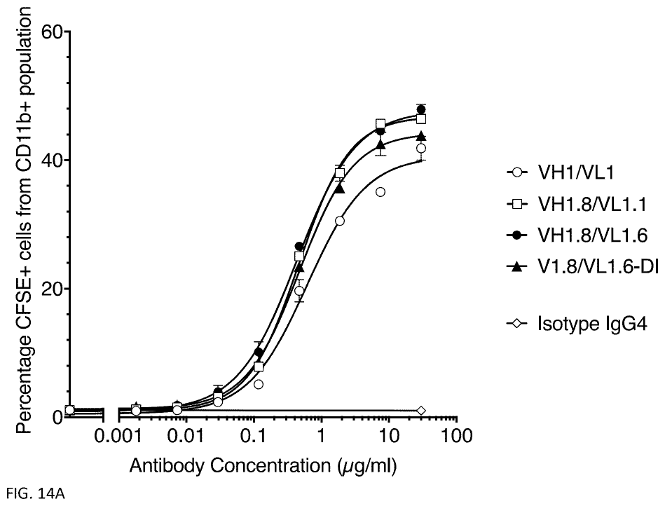

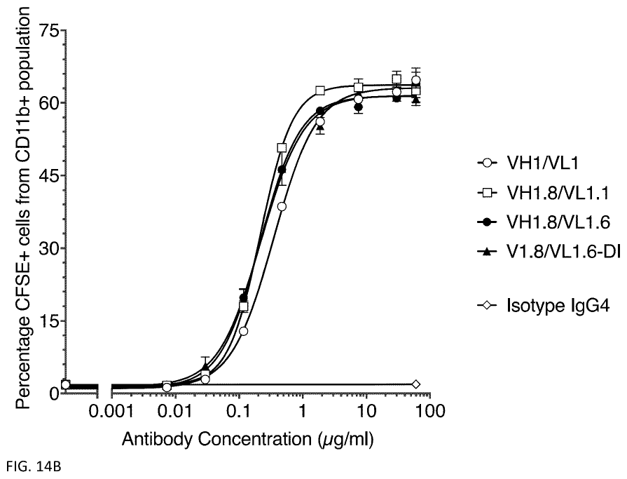

Finally, two remaining HAF peptides were found in the VL sequences VL1.1-1.9. The first HAF peptide ‘LIYKGSNRF’ (SEQ ID NO:112), spanning the Framework2/LCDR2 regions was identified in the analysis shown in FIG. 13A. The second HAF peptide ‘VGVYYCFQA’ (SEQ ID NO:113), spanning the Framework3/LCDR3 regions was identified in the analysis shown in FIG. 13A. Both HAF peptides could be fully ablated by mutation of the residues at position 1 to alanine (L>A and V>A, respectively). To sample these substitutions, a new clone ‘VH1.8/VL1.6-DI’ was constructed, containing both alanine substitutions in the VL domain.

### Potency and Affinity Analyses of Key Leads

Clones h5F9G4, VH1.8/VL1.1, VH1.8/VL1.6, and VH1.8/VL1.6-DI were readily expressed and purified in the human IgG4 (S228P) format. For all clones the Fab domains were also expressed as soluble monomers and purified for binding affinity analyses. These Fabs were then examined for affinity to human and cyno CD47 by a biosensor system sold under the trademark Biacore®, using steady state affinity analysis. Steady state was used as the binding affinity analysis as the on and off rates of all Fabs were too fast for reliable 1:1 modelling. As shown in Table 6, all 4 clones exhibited accurate, well-fitted data, as evidenced by very low Chi2 values. All clones were found to have binding affinities in the low nM range, within 2-fold of h5F9G4.

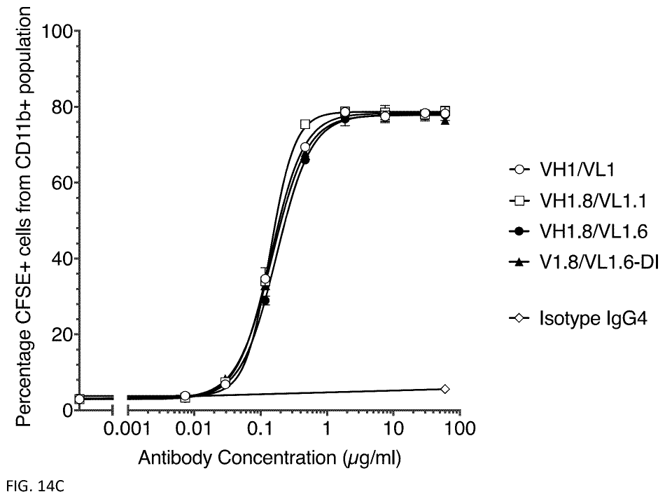

As final analysis of retained potency, clones h5F9G4, VH1.8/VL1.1, VH1.8/VL1.6, and VH1.8/VL1.6-DI in IgG4 (S228P) format were examined for their relative potencies in a flow cytometric assay of phagocytosis, using human monocyte-derived macrophages derived from 3 separate donors and Jurkat human cancer cells as the CD47+target cells. For all clones analysed against cells from Donor 1 (FIG. 14A), Donor 2 (FIG. 14B) and Donor 3 (FIG. 14C), all clones had fully retained or improved over the potency of h5F9G4, as evidenced by fully overlapping response curves.

Although the present invention has been described with reference to preferred or exemplary embodiments, those skilled in the art will recognize that various modifications and variations to the same can be accomplished without departing from the spirit and scope of the present invention and that such modifications are clearly contemplated herein. No limitation with respect to the specific embodiments disclosed herein and set forth in the appended claims is intended nor should any be inferred.

No limitation with respect to the specific embodiments disclosed herein and set forth in the appended claims is intended nor should any be inferred. All documents, or portions of documents, cited herein, including but not limited to patents, patent applications, articles, books, and treatises, are hereby expressly incorporated by reference in their entirety for any purpose. In the event that one or more of the incorporated documents or portions of documents define a term that contradicts that term's definition in the application, the definition that appears in this application controls. However, mention of any reference, article, publication, patent, patent publication, and patent application cited herein is not, and should not be taken as an acknowledgment, or any form of suggestion, that they constitute valid prior art or form part of the common general knowledge in any country in the world.

TABLE 1
Amino acid sequences murine anti-CD47 CDRs as defined here
(“Unified” scheme) in comparison to alternative definitions.

| Scheme | HCDR1 | HCDR2 | HCDR3 | LCDR1 | LCDR2 | LCDR3 |
|---|---|---|---|---|---|---|
| Unified | GYTFTNYN | MGTIYPGNDDTSYNQK | GGYRAMDY | RSSQSIVYSNGNTYLG | KVSNRFS | FQGSHVPY |
| | MH | FKD | (SEQ ID | (SEQ ID NO: 130) | (SEQ ID | T |
| | (SEQ ID | (SEQ ID NO: 119) | NO: 126) | | NO: 134) | (SEQ ID |
| | NO: 114) | | | | | NO: 136) |
| Kabat | NYNMH | TIYPGNDDTSYNQKFK | GGYRAMDY | RSSQSIVYSNGNTYLG | KVSNRFS | FQGSHVPY |
| | (SEQ ID | D | (SEQ ID | (SEQ ID NO: 130) | (SEQ ID | T |
| | NO: 115) | (SEQ ID NO: 120) | NO: 126) | | NO: 134) | (SEQ ID |
| | | | | | | NO: 136) |
| Chotia | GYTFTNY | YPGNDD | GGYRAMDY | RSSQSIVYSNGNTYLG | KVSNRFS | FQGSHVPY |
| | (SEQ ID | (SEQ ID NO: 121) | (SEQ ID | (SEQ ID NO: 130) | (SEQ ID | T |
| | NO: 116) | | NO: 126) | | NO: 134) | (SEQ ID |
| | | | | | | NO: 136) |
| IMGT | GYTFTNYN | IYPGNDDT | ARGGYRAM | QSIVYSNGNTY | KVS | FQGSHVPY |
| | (SEQ ID | (SEQ ID NO: 122) | DY | (SEQ ID NO: 131) | | T |
| | NO: 117) | | (SEQ ID | | | (SEQ ID |
| | | | NO: 127) | | | NO: 136) |
| AHo | GYTFTNYN | IYPGNDDTSYNQKFKD | GGYRAMD | SSQSIVYSNGNTY | KVSNRFS | GSHVPY |
| | MH | (SEQ ID NO: 123) | (SEQ ID | (SEQ ID NO: 132) | (SEQ ID | (SEQ ID |
| | (SEQ ID | | NO: 128) | | NO: 134) | NO: 137) |
| | NO: 114) | | | | | |
| AbM | GYTFTNYN | TIYPGNDDTS | GGYRAMDY | RSSQSIVYSNGNTYLG | KVSNRFS | FQGSHVPY |
| | MH | (SEQ ID NO: 124) | (SEQ ID | (SEQ ID NO: 130) | (SEQ ID | T |
| | (SEQ ID | | NO: 126) | | NO: 134) | (SEQ ID |
| | NO: 114) | | | | | NO: 136) |
| Contact | TNYNMH | MGTIYPGNDDTS | ARGGYRAM | VYSNGNTYLGWY | ALIYKVSN | FQGSHVPY |
| | (SEQ ID | (SEQ ID NO: 125) | D | (SEQ ID NO: 133) | RF | (SEQ ID |
| | NO: 118) | | (SEQ ID | | (SEQ ID | NO: 138) |
| | | | NO: 129) | | NO: 135) | |

TABLE 2
Amino acid sequence of
5F9G4 murine anti-CD47 v-domains (m5F9G4)
and human germline CDR grafts (h5F9G4).

| | Human | |
|---|---|---|
| V DOMAIN | germline¹ | Amino acid sequence² |
| m5F9G4-VH | na | QVQLQQPGAELVKPGASVMM |
| | | SCKASGYTFTNYNMHWVKQT |
| | | PGQGLEWIGTIYPGNDDTSY |
| | | NQKFKDKATLTADKSSSAAY |
| | | MQLSSLTSEDSAVYYCARGG |
| | | YRAMDYWGQGTSVTVSS |
| | | (SEQ ID NO: 139) |
| h5F9G4-VH | IGHV1-3 | QVQLVQSGAEVKKPGASVKV |
| | | SCKASGYTFTNYNMHWVRQA |
| | | PGQRLEWMGTIYPGNDDTSY |
| | | NQKFKDRVTITADTSASTAY |
| | | MELSSLRSEDTAVYYCARGG |
| | | YRAMDYWGQGTLVTVSS |
| | | (SEQ ID NO: 140) |
| VH graft | IGHV5-51³ | EVQLVQSGAEVKKPGESLKI |
| | | SCKGSGYSFTNYNMHWVRQM |
| | | PGKGLEWMGTIYPGNDDTSY |
| | | NQKFQGQVTISADKSISTAY |
| | | LQWSSLKASDTAMYYCARGG |
| | | YRAMDYWGQGTLVTVSS |
| | | (SEQ ID NO: 141) |
| m5F9G4-VL | na | DVLMTQTPLSLPVSLGDQAS |
| | | ISCRSSQSIVYSNGNTYLGW |
| | | YLQKPGQSPKLLIYKVSNRF |
| | | SGVPDRFSGSGSGTDFTLKI |
| | | SRVEAEDLGVYHCFQGSHVP |
| | | YTFGGGTKVEIK |
| | | (SEQ ID NO: 142) |
| h5F9G4-VL | IGKV2-28 | DIVMTQSPLSLPVTPGEPAS |
| | | ISCRSSQSIVYSNGNTYLGW |
| | | YLQKPGQSPQLLIYKVSNRF |
| | | SGVPDRFSGSGSGTDFTLKI |
| | | SRVEAEDVGVYYCFQGSHVP |
| | | YTFGQGTKLEIK |
| | | (SEQ ID NO: 143) |
| VL graft | IGKV2-28³ | DIVMTQSPLSLPVTPGEPAS |
| | | ISCRSSQSIVYSNGNTYLGW |
| | | YLQKPGQSPQLLIYKVSNRF |
| | | SGVPDRFSGSGSGTDFTLKI |
| | | SRVEAEDVGVYYCFQGSHVP |
| | | YTFGQGTKLEIK |
| | | (SEQ ID NO: 144) |

¹Human germline definitions used for grafting, based on IMGT system.
²CDR residues are in bold and underlined. As noted above, the “Unified” CDR definitions used in this manuscript are an expanded definition in comparison to the classical Kabat definition. Each sequence above shows the framework regions (FRs) and the CDRs in the following order: FR1-CDR1-FR2-CDR2-FR3-CDR3-FR4.
³Grafts are fully germline in the framework regions, used as the template for CDR mutant library construction.

TABLE 3
Amino acid sequences of unique CDRs from 269 unique
anti-CD47 v-domains in the IGKV2-28/IGHV5-51 background.

| LCDR1 | LCDR2 | LCDR3 | HCDR1 | HCDR2 | HCDR2 | HCDR2 | HCDR3 |
|---|---|---|---|---|---|---|---|
| RSSQSLLH | KGSNRFS | FQALHVPYT | GYSFTNYNIG | IGTIYPGDDD | MGTIYPGDDD | MGTIYPGNDD | GGARAMDY |
| SGGNTYLD | (SEQ ID | (SEQ ID | (SEQ ID | TRYNPKFQG | TSYSQKFQG | TSYNQSFQG | (SEQ ID |
| (SEQ ID | NO: 52) | NO: 165) | NO: 195) | (SEQ ID | (SEQ ID | (SEQ ID | NO: 289) |
| NO: 145) | | | | NO: 200) | NO: 232) | NO: 262) | |
| RSSQSLLH | KVSNRAS | FQAMQTPYT | GYSFTNYNIH | MGIIYPGDDD | MGTIYPGDGD | MGTIYPGNDD | GGCRALDY |
| SGGNTYLG | (SEQ ID | (SEQ ID | (SEQ ID | TRYNPKFQG | TSYNPSFQG | TSYSPKFQG | (SEQ ID |
| (SEQ ID | NO: 163) | NO: 166) | NO: 57) | (SEQ ID | (SEQ ID | (SEQ ID | NO: 290) |
| NO: 146) | | | | NO: 201) | NO: 233) | NO: 263) | |
| RSSQSLLH | KVSNRSS | FQASHIPYT | GYSFTNYNMH | MGIIYPGDDD | MGTIYPGDND | MGTIYPGNDD | GGCRAMDY |
| SGYNTYLG | (SEQ ID | (SEQ ID | (SEQ ID | TRYNPSFQG | TSYNPSFQG | TSYSPRFQG | (SEQ ID |
| (SEQ ID | NO: 164) | NO: 167) | NO: 196) | (SEQ ID | (SEQ ID | (SEQ ID | NO: 291) |
| NO: 147) | | | | NO: 202) | NO: 234) | NO: 264) | |
| RSSQSLLH | | FQASHTPYT | GYSFTNYSIH | MGIIYPGDDD | MGTIYPGDSD | MGTIYPGNDD | GGCRAMGY |
| SNGNTYLD | | (SEQ ID | (SEQ ID | TSYNPKFQG | TKYNPKFQG | TSYSPSFQG | (SEQ ID |
| (SEQ ID | | NO: 168) | NO: 197) | (SEQ ID | (SEQ ID | (SEQ ID | NO: 292) |
| NO: 148) | | | | NO: 203) | NO: 235) | NO: 265) | |
| RSSQSLLH | | FQASHVPYT | GYSFTSYNIH | MGIIYPGDDD | MGTIYPGDSD | MGTIYPGNDD | GGDRAMDY |
| SNGNTYLG | | (SEQ ID | (SEQ ID | TSYNQKFQG | TRYNPKFQG | TSYSQKFQG | (SEQ ID |
| (SEQ ID | | NO: 53) | NO: 198) | (SEQ ID | (SEQ ID | (SEQ ID | NO: 293) |
| NO: 51) | | | | NO: 204) | NO: 58) | NO: 90) | |
| RSSQSLLH | | FQASQIPYT | GYSFTSYNMH | MGIIYPGDSD | MGTIYPGDSD | MGTIYPGNDD | GGERAMDY |
| SNYNTYLG | | (SEQ ID | (SEQ ID | TRYNPKFQG | TRYNPSFQG | TSYSQSFQG | (SEQ ID |
| (SEQ ID | | NO: 169) | NO: 199) | (SEQ ID | (SEQ ID | (SEQ ID | NO: 294) |
| NO: 149) | | | | NO: 205) | NO: 236) | NO: 266) | |
| RSSQSLLQ | | FQASQTPYT | | MGIIYPGDSD | MGTIYPGDSD | MGTIYPGNDG | GGFRAEDY |
| SNGNTYLG | | (SEQ ID | | TRYNPSFQG | TRYNQKFQG | TRYNQKFQG | (SEQ ID |
| (SEQ ID | | NO: 170) | | (SEQ ID | (SEQ ID | (SEQ ID | NO: 295) |
| NO: 150) | | | | NO: 206) | NO: 98) | NO: 267) | |
| RSSQSLLY | | FQASQVPYT | | MGIIYPGDSD | MGTIYPGDSD | MGTIYPGNGD | GGFRAMDY |
| SGGNTYLG | | (SEQ ID | | TRYNQKFQG | TRYSPKFQG | TSYNHKFQG | (SEQ ID |
| (SEQ ID | | NO: 54) | | (SEQ ID | (SEQ ID | (SEQ ID | NO: 50) |
| NO: 151) | | | | NO: 207) | NO: 237) | NO: 268) | |
| RSSQSLLY | | FQASRVPYT | | MGIIYPGDSD | MGTIYPGDSD | MGTIYPGNGD | GGFRAQDY |
| SNGNTYLG | | (SEQ ID | | TSYNQSFQG | TRYSPRFQG | TSYNQKFQG | (SEQ ID |
| (SEQ ID | | NO: 171) | | (SEQ ID | (SEQ ID | (SEQ ID | NO: 296) |
| NO: 152) | | | | NO: 208) | NO: 238) | NO: 269) | |
| RSSQSLVH | | FQATQVPYT | | MGIIYPGNDD | MGTIYPGDSD | MGTIYPGNND | GGGRAMDY |
| SGGNTYLG | | (SEQ ID | | TRYNPKFQG | TRYSPSFQG | TSYNPKFQG | (SEQ ID |
| (SEQ ID | | NO: 172) | | (SEQ ID | (SEQ ID | (SEQ ID | NO: 297) |
| NO: 153) | | | | NO: 209) | NO: 239) | NO: 270) | |
| RSSQSLVH | | FQGLQVPYT | | MGIIYPGNDD | MGTIYPGDSD | MGTIYPGNSD | GGHRAEDY |
| SGYNTYLG | | (SEQ ID | | TRYNPSFQG | TRYSQKFQG | TRYNHSFQG | (SEQ ID |
| (SEQ ID | | NO: 173) | | (SEQ ID | (SEQ ID | (SEQ ID | NO: 298) |
| NO: 154) | | | | NO: 210) | NO: 240) | NO: 271) | |
| RSSQSLVH | | FQGSHIPYT | | MGIIYPGNDD | MGTIYPGDSD | MGTIYPGNSD | GGHRAMDY |
| SNGNTYLD | | (SEQ ID | | TRYNQKFQG | TRYSQSFQG | TRYNPKFQG | (SEQ ID |
| (SEQ ID | | NO: 174) | | (SEQ ID | (SEQ ID | (SEQ ID | NO: 299) |
| NO: 155) | | | | NO: 211) | NO: 241) | NO: 101) | |
| RSSQSLVH | | FQGSHTPYT | | MGIIYPGNSD | MGTIYPGDSD | MGTIYPGNSD | GGIRAMDY |
| SNGNTYLG | | (SEQ ID | | TRYNPKFQG | TSYNHKFQG | TRYNPSFQG | (SEQ ID |
| (SEQ ID | | NO: 175) | | (SEQ ID | (SEQ ID | (SEQ ID | NO: 99) |
| NO: 156) | | | | NO: 212) | NO: 242) | NO: 272) | |
| RSSQSLVH | | FQGSHVPYI | | MGIIYPGNSD | MGTIYPGDSD | MGTIYPGNSD | GGKRAMDY |
| SNGQTYLG | | (SEQ ID | | TRYNPSFQG | TSYNPKFHG | TRYNQKFQG | (SEQ ID |
| (SEQ ID | | NO: 100) | | (SEQ ID | (SEQ ID | (SEQ ID | NO: 93) |
| NO: 157) | | | | NO: 213) | NO: 243) | NO: 273) | |
| RSSQSLVH | | FQGSQTPYT | | MGIIYPGNSD | MGTIYPGDSD | MGTIYPGNSD | GGLRAMDY |
| SNYNTYLG | | (SEQ ID | | TRYNQKFQG | TSYNPKFQG | TRYNQSFQG | (SEQ ID |
| (SEQ ID | | NO: 176) | | (SEQ ID | (SEQ ID | (SEQ ID | NO: 300) |
| NO: 158) | | | | NO: 214) | NO: 94) | NO: 274) | |
| RSSQSLVH | | FQGSQVPYT | | MGIIYPGNSD | MGTIYPGDSD | MGTIYPGNSD | GGLRTMDY |
| SSGNTYLG | | (SEQ ID | | TRYNQRFQG | TSYNPRFQG | TRYSPKFQG | (SEQ ID |
| (SEQ ID | | NO: 177) | | (SEQ ID | (SEQ ID | (SEQ ID | NO: 301) |
| NO: 159) | | | | NO: 215) | NO: 244) | NO: 275) | |
| RSSQSLVQ | | LQGSHIPYT | | MGIIYPGNSD | MGTIYPGDSD | MGTIYPGNSD | GGMRAMDY |
| SNGNTYLG | | (SEQ ID | | TRYNQSFQG | TSYNPSFQG | TRYSPSFQG | (SEQ ID |
| (SEQ ID | | NO: 178) | | (SEQ ID | (SEQ ID | (SEQ ID | NO: 302) |
| NO: 160) | | | | NO: 216) | NO: 245) | NO: 276) | |
| RSSQSLVY | | LQGSHTPYT | | MGIIYPGNSD | MGTIYPGDSD | MGTIYPGNSD | GGNRAKDY |
| SNGNTYLG | | (SEQ ID | | TRYSPKFQG | TSYNQKFQG | TRYSQKFQG | (SEQ ID |
| (SEQ ID | | NO: 179) | | (SEQ ID | (SEQ ID | (SEQ ID | NO: 303) |
| NO: 161) | | | | NO: 217) | NO: 246) | NO: 277) | |
| RSSQSLVY | | LQGSHVPYT | | MGIIYPGNSD | MGTIYPGDSD | MGTIYPGNSD | GGNRAMDY |
| SNYNTYLG | | (SEQ ID | | TRYSPSFQG | TSYNQSFQG | TSYNPKFQG | (SEQ ID |
| (SEQ ID | | NO: 180) | | (SEQ ID | (SEQ ID | (SEQ ID | NO: 304) |
| NO: 162) | | | | NO: 218) | NO: 247) | NO: 92) | |
| | | LQGSQTPYT | | MGIIYPGNSD | MGTIYPGDSD | MGTIYPGNSD | GGQRAMDY |
| | | (SEQ ID | | TSYNPKFQG | TSYSPKFQG | TSYNPRFQG | (SEQ ID |
| | | NO: 181) | | (SEQ ID | (SEQ ID | (SEQ ID | NO: 305) |
| | | | | NO: 219) | NO: 248) | NO: 278) | |
| | | LQGSQVPYT | | MGIIYPGNSD | MGTIYPGDSD | MGTIYPGNSD | GGRRAMDY |
| | | (SEQ ID | | TSYNPRFQG | TSYSPSFQG | TSYNPSFQG | (SEQ ID |
| | | NO: 182) | | (SEQ ID | (SEQ ID | (SEQ ID | NO: 306) |
| | | | | NO: 220) | NO: 249) | NO: 61) | |
| | | MQALQVPYT | | MGIIYPGNSD | MGTIYPGDSD | MGTIYPGNSD | GGSRAKDY |
| | | (SEQ ID | | TSYNPSFQG | TSYSQKFQG | TSYNQKFQG | (SEQ ID |
| | | NO: 183) | | (SEQ ID | (SEQ ID | (SEQ ID | NO: 307) |
| | | | | NO: 221) | NO: 250) | NO: 279) | |
| | | MQASHIPYT | | MGIIYPGNSD | MGTIYPGNDD | MGTIYPGNSD | GGSRAMDY |
| | | (SEQ ID | | TSYNQKFQG | TKYSPKFQG | TSYNQSFQG | (SEQ ID |
| | | NO: 184) | | (SEQ ID | (SEQ ID | (SEQ ID | NO: 308) |
| | | | | NO: 222) | NO: 251) | NO: 280) | |
| | | MQASHTPYT | | MGIIYPGNSD | MGTIYPGNDD | MGTIYPGNSD | GGTRAMDY |
| | | (SEQ ID | | TSYSPKFQG | TRYNPKFQG | TSYSPKFQG | (SEQ ID |
| | | NO: 185) | | (SEQ ID | (SEQ ID | (SEQ ID | NO: 309) |
| | | | | NO: 223) | NO: 252) | NO: 281) | |
| | | MQASHVPYT | | MGTIYPGDDD | MGTIYPGNDD | MGTIYPGNSD | GGVRAMDY |
| | | (SEQ ID | | TRYNPKFQG | TRYNPSFQG | TSYSPSFQG | (SEQ ID |
| | | NO: 96) | | (SEQ ID | (SEQ ID | (SEQ ID | NO: 62) |
| | | | | NO: 224) | NO: 253) | NO: 282) | |
| | | MQASQIPYT | | MGTIYPGDDD | MGTIYPGNDD | MGTIYPGNSD | GGVRATDY |
| | | (SEQ ID | | TRYNPSFQG | TRYNQKFQG | TSYSQKFQG | (SEQ ID |
| | | NO: 186) | | (SEQ ID | (SEQ ID | (SEQ ID | NO: 310) |
| | | | | NO: 225) | NO: 254) | NO: 283) | |
| | | MQASQTPYT | | MGTIYPGDDD | MGTIYPGNDD | MGTIYSGNSD | GGWRAMDY |
| | | (SEQ ID | | TRYNQKFQG | TRYNQSFQG | TSYSPSFQG | (SEQ ID |
| | | NO: 187) | | (SEQ ID | (SEQ ID | (SEQ ID | NO: 311) |
| | | | | NO: 226) | NO: 255) | NO: 284) | |
| | | MQASQVPYT | | MGTIYPGDDD | MGTIYPGNDD | MGTIYPGNDD | GGYHAMDY |
| | | (SEQ ID | | TRYNQNFQG | TRYSPKFQG | TRYSQSFQG | (SEQ ID |
| | | NO: 63) | | (SEQ ID | (SEQ ID | (SEQ ID | NO: 312) |
| | | | | NO: 227) | NO: 256) | NO: 285) | |
| | | MQGLHVPYT | | MGTIYPGDDD | MGTIYPGNDD | MGTIYPGNDD | GGYKAMDY |
| | | (SEQ ID | | TRYNQSFQG | TRYSPSFQG | TSYNHKFQG | (SEQ ID |
| | | NO: 188) | | (SEQ ID | (SEQ ID | (SEQ ID | NO: 313) |
| | | | | NO: 228) | NO: 257) | NO: 286) | |
| | | MQGLQVPYT | | MGTIYPGDDD | MGTIYPGNDD | MGTIYPGNDD | GGYRAADY |
| | | (SEQ ID | | TRYSPKFQG | TRYSQKFQG | TSYNPKFQG | (SEQ ID |
| | | NO: 189) | | (SEQ ID | (SEQ ID | (SEQ ID | NO: 314) |
| | | | | NO: 229) | NO: 258) | NO: 287) | |
| | | MQGSHIPYT | | MGTIYPGDDD | MGTIYPGDDD | MGTIYPGNDD | GGYRAEDY |
| | | (SEQ ID | | TRYSPSFQG | TSYNPKFQG | TSYNPSFQG | (SEQ ID |
| | | NO: 190) | | (SEQ ID | (SEQ ID | (SEQ ID | NO: 59) |
| | | | | NO: 230) | NO: 259) | NO: 288) | |
| | | MQGSHTPYT | | MGTIYPGDDD | MGTIYPGDDD | | GGYRALDY |
| | | (SEQ ID | | TRYSQKFQG | TSYNPSFQG | | (SEQ ID |
| | | NO: 191) | | (SEQ ID | (SEQ ID | | NO: 315) |
| | | | | NO: 231) | NO: 260) | | |
| | | MQGSHVPYT | | | MGTIYPGDDD | | GGYRAPDY |
| | | (SEQ ID | | | TSYNQKFQG | | (SEQ ID |
| | | NO: 97) | | | (SEQ ID | | NO: 316) |
| | | | | | NO: 261) | | |
| | | MQGSQIPYT | | | | | GGYRAQDY |
| | | (SEQ ID | | | | | (SEQ ID |
| | | NO: 192) | | | | | NO: 95) |
| | | MQGSQTPYT | | | | | GGYRARDY |
| | | (SEQ ID | | | | | (SEQ ID |
| | | NO: 193) | | | | | NO: 317) |
| | | MQGSQVPYT | | | | | GGYRASDY |
| | | (SEQ ID | | | | | (SEQ ID |
| | | NO: 194) | | | | | NO: 318) |
| | | | | | | | GGYRATDY |
| | | | | | | | (SEQ ID |
| | | | | | | | NO: 319) |
| | | | | | | | GGYRAVDY |
| | | | | | | | (SEQ ID |
| | | | | | | | NO: 320) |
| | | | | | | | GGYRIMDY |
| | | | | | | | (SEQ ID |
| | | | | | | | NO: 321) |
| | | | | | | | GGYRTEDY |
| | | | | | | | (SEQ ID |
| | | | | | | | NO: 322) |
| | | | | | | | GGYRTMDY |
| | | | | | | | (SEQ ID |
| | | | | | | | NO: 323) |
| | | | | | | | GGYRTTDY |
| | | | | | | | (SEQ ID |
| | | | | | | | NO: 324) |
| | | | | | | | GGYRVMDY |
| | | | | | | | (SEQ ID |
| | | | | | | | NO: 325) |
| | | | | | | | GGYRWMDY |
| | | | | | | | (SEQ ID |
| | | | | | | | NO: 326) |
| | | | | | | | QGYRAMDY |
| | | | | | | | (SEQ ID |
| | | | | | | | NO: 327) |

TABLE 4
Amino acid sequences of CDRs of unique, library-derived and
designer, human/cyno cross-reactive anti-CD47 IgGs.

| Clone | | | | | | |
|---|---|---|---|---|---|---|
| name | LCDR1 | LCDR2 | LCDR3 | HCRD1 | HCDR2 | HCDR3 |
| F-E5 | RSSQSLLHSNGNTYLG | KGSNRFS | MQASQVPYT | GYSFTNYNIH | MGTIYPGNSDTSYNPKFQG | GGKRAMDY |
| | (SEQ ID NO: 51) | (SEQ ID | (SEQ ID | (SEQ ID NO: 57) | (SEQ ID NO: 92) | (SEQ ID |
| | | NO: 52) | NO: 63) | | | NO: 93) |
| A-H3 | RSSQSLLHSKGNTYLG | KGSNRFS | MQASQVPYT | GYSFTNYNIH | MGTIYPGNSDTSYNPSFQG | GGVRAMDY |
| | (SEQ ID NO: 328) | (SEQ ID | (SEQ ID | (SEQ ID NO: 57) | (SEQ ID NO: 61) | (SEQ ID |
| | | NO: 52) | NO: 63) | | | NO: 62) |
| C-D5 | RSSQSLLHSNYNTYLG | KGSNRFS | FQGSQTPYT | GYSFTNYNIH | MGTIYPGDSDTRYNPKFQG | GGIRAMDY |
| | (SEQ ID NO: 329) | (SEQ ID | (SEQ ID | (SEQ ID NO: 57) | (SEQ ID NO: 58) | (SEQ ID |
| | | NO: 52) | NO: 176) | | | NO: 99) |
| E-C5 | RSSQSLLHSNGNTYLG | KGSNRFS | MQASHVPYT | GYSFTNYNIH | MGTIYPGDSDTSYNPKFQG | GGYRAQDY |
| | (SEQ ID NO: 51) | (SEQ ID | (SEQ ID | (SEQ ID NO: 57) | (SEQ ID NO: 94) | (SEQ ID |
| | | NO: 52) | NO: 96) | | | NO: 95) |
| E-D6 | RSSQSLLHSNGNTYLG | KGSNRFS | MQGSHVPYT | GYSFTNYNIH | MGTIYPGDSDTSYNPKFQG | GGYRAEDY |
| | (SEQ ID NO: 51) | (SEQ ID | (SEQ ID | (SEQ ID NO: 57) | (SEQ ID NO: 94) | (SEQ ID |
| | | NO: 52) | NO: 97) | | | NO: 59) |
| B-A6 | RSSQSLLHSNGNTYLG | KGSNRFS | FQGSHVPYI | GYSFTNYNIH | MGTIYPGDSDTRYNQKFQG | GGIRAMDY |
| | (SEQ ID NO: 51) | (SEQ ID | (SEQ ID | (SEQ ID NO: 57) | (SEQ ID NO: 98) | (SEQ ID |
| | | NO: 52) | NO: 100) | | | NO: 99) |
| D-C12 | RSSQSLLHSNGNTYLG | KGSNRFS | MQGSHVPYT | GYSFTNYNIH | MGTIYPGNSDTRYNPKFQG | GGFRAMDY |
| | (SEQ ID NO: 51) | (SEQ ID | (SEQ ID | (SEQ ID NO: 57) | (SEQ ID NO: 101) | (SEQ ID |
| | | NO: 52) | NO: 97) | | | NO: 50) |
| B-D2 | RSSQSLLHSNGNTYLG | KGSNRFS | FQASQVPYT | GYSFTNYNIH | MGTIYPGNSDTRYNPSFQG | GGERAMDY |
| | (SEQ ID NO: 51) | (SEQ ID | (SEQ ID | (SEQ ID NO: 57) | (SEQ ID NO: 272) | (SEQ ID |
| | | NO: 52) | NO: 54) | | | NO: 294) |
| F-G3 | RSSQSLLHSNGNTYLG | KGSNRFS | MQASQVPYT | GYSFTNYNIH | MGTIYPGNSDTSYNHKFQG | GGNRAEDY |
| | (SEQ ID NO: 51) | (SEQ ID | (SEQ ID | (SEQ ID NO: 57) | (SEQ ID NO: 331) | (SEQ ID |
| | | NO: 52) | NO: 63) | | | NO: 333) |
| E-A1 | RSSQSLLHSNGNTYLG | KGSNRFS | MQGSHVPYT | GYSFTNYNIH | MGTIYPGDSDTRYNPKFQG | GGYRAEDY |
| | (SEQ ID NO: 51) | (SEQ ID | (SEQ ID | (SEQ ID NO: 57) | (SEQ ID NO: 58) | (SEQ ID |
| | | NO: 52) | NO: 97) | | | NO: 59) |
| D6 | RSSQSLLHSNGNTYLG | KGSNRFS | MQASQVPYT | GYSFTNYNIH | MGTIYPGNSDTSYNPSFQG | GGVRAMDY |
| | (SEQ ID NO: 51) | (SEQ ID | (SEQ ID | (SEQ ID NO: 57) | (SEQ ID NO: 61) | (SEQ ID |
| | | NO: 52) | NO: 63) | | | NO: 62) |
| VH1.1 | | | | GYTFTNYAMH | MGTIYPGNDDTKYNQKFQG | GGYRAEDY |
| | | | | (SEQ ID NO: 48) | (SEQ ID NO: 49) | (SEQ ID |
| | | | | | | NO: 59) |
| VH1.2 | | | | GYTFTDYNMH | MGTIYPGNDDTKYNQKFQG | GGYRAEDY |
| | | | | (SEQ ID NO: 88) | (SEQ ID NO: 49) | (SEQ ID |
| | | | | | | NO: 59) |
| VH1.3 | | | | GYTFTDYNMH | MGTIYPGNDDTKYSQKFQG | GGYRAEDY |
| | | | | (SEQ ID NO: 88) | (SEQ ID NO: 91) | (SEQ ID |
| | | | | | | NO: 59) |
| VH1.4 | | | | GYTFTDYNMH | MGTIYPGNDDTKYSQKFQG | GGFRAMDY |
| | | | | (SEQ ID NO: 88) | (SEQ ID NO: 91) | (SEQ ID |
| | | | | | | NO: 50) |
| VH1.5 | | | | GYTFTDYNMH | MGTIYPGNDDTKYNQKFQG | GGFRAMDY |
| | | | | (SEQ ID NO: 88) | (SEQ ID NO: 49) | (SEQ ID |
| | | | | | | NO: 50) |
| VH1.6 | | | | GYTFTNYAMH | MGTIYPGNDDTSYSQKFQG | GGYRAEDY |
| | | | | (SEQ ID NO: 48) | (SEQ ID NO: 90) | (SEQ ID |
| | | | | | | NO: 59) |
| VH1.7 | | | | GYTFTNYNIH | MGTIYPGNDDTSYSQKFQG | GGFRAMDY |
| | | | | (SEQ ID NO: 89) | (SEQ ID NO: 90 | (SEQ ID |
| | | | | | | NO: 50) |
| VH1.8 | | | | GYTFTNYAMH | MGTIYPGNDDTKYNQKFQG | GGFRAMDY |
| | | | | (SEQ ID NO: 48) | (SEQ ID NO: 49) | (SEQ ID |
| | | | | | | NO: 50) |
| VH1.9 | | | | GYTFTNYAMH | MGTIYPGNDDTKYSQKFQG | GGYRAEDY |
| | | | | (SEQ ID NO: 48) | (SEQ ID NO: 91) | (SEQ ID |
| | | | | | | NO: 59) |
| VH1.10 | | | | GYTFTDYNMH | MGTIYPGNDDTSYSQKFQG | GGFRAMDY |
| | | | | (SEQ ID NO: 88) | (SEQ ID NO: 90) | (SEQ ID |
| | | | | | | NO: 50) |
| VH1.11 | | | | GYTFTNYAMH | MGTIYPGNDDTKYSQKFQG | GGFRAMDY |
| | | | | (SEQ ID NO: 48) | (SEQ ID NO: 91) | (SEQ ID |
| | | | | | | NO: 50) |
| VH1.12 | | | | GYTFTNYAMH | MGTIYPGNDNTKYNQKFQG | GGFRAMDY |
| | | | | (SEQ ID NO: 48) | (SEQ ID NO: 332) | (SEQ ID |
| | | | | | | NO: 50) |
| VL1.1 | RSSQSLLHSNGNTYLG | KGSNRFS | FQASHVPYT | | | |
| | (SEQ ID NO: 51) | (SEQ ID | (SEQ ID | | | |
| | | NO: 52) | NO: 53) | | | |
| VL1.2 | RSSQSLLHSNGNTYLG | KGSNRFS | FQASQVPYT | | | |
| | (SEQ ID NO: 51) | (SEQ ID | (SEQ ID | | | |
| | | NO: 52) | NO: 54) | | | |
| VL1.3 | RSSQSLLHSNANTYLG | KGSNRFS | FQASQVPYT | | | |
| | (SEQ ID NO: 55) | (SEQ ID | (SEQ ID | | | |
| | | NO: 52) | NO: 54) | | | |
| VL1.4 | RSSQSLLHSNAYNYLG | KGSNRFS | FQASQVPYT | | | |
| | (SEQ ID NO: 56) | (SEQ ID | (SEQ ID | | | |
| | | NO: 52) | NO: 54) | | | |
| VL1.5 | RSSQSLLHSNAYNYLG | KGSNRFS | FQASHVPYT | | | |
| | (SEQ ID NO: 56) | (SEQ ID | (SEQ ID | | | |
| | | NO: 52) | NO: 53) | | | |
| VL1.6 | RSSQSLLHSNANTYLG | KGSNRFS | FQASHVPYT | | | |
| | (SEQ ID NO: 55) | (SEQ ID | (SEQ ID | | | |
| | | NO: 52) | NO: 53) | | | |
| VL1.7 | RSSQSLLHSNAYNYLG | KGSNRAS | FQASHVPYT | | | |
| | (SEQ ID NO: 56) | (SEQ ID | (SEQ ID | | | |
| | | NO: 330) | NO: 53) | | | |
| VL1.8 | RSSQSLLHSNAYNYLG | KGSNRFS | MQASHVPYT | | | |
| | (SEQ ID NO: 56) | (SEQ ID | (SEQ ID | | | |
| | | NO: 52) | NO: 96) | | | |
| VL1.9 | RSSQSLLHSNAYNYLG | KGSNRAS | MQASHVPYT | | | |
| | (SEQ ID NO: 56) | (SEQ ID | (SEQ ID | | | |
| | | NO: 330) | NO: 96) | | | |

TABLE 5
Assay technology known under the trademark HTRF ®
epitope competition IC50 values for designer lead IgGs.

| | IC50 μg/ml | |
|---|---|---|
| Antibody | hCD47 | cCD47 |
| m5F9G4 | 0.601 | 0.809 |
| h5F9G4 | 0.464 | 0.899 |
| VH1.8/VL1.1 | 0.459 | 0.629 |
| VH1.8/VL1.2 | 0.586 | 0.824 |
| VH1.8/VL1.3 | 0.928 | 1.327 |
| VH1.8/VL1.4 | 2.944 | 5.169 |
| VH1.8/VL1.5 | 0.911 | 1.36 |
| VH1.8/VL1.6 | 0.56 | 0.626 |
| VH1.1/VL1.6 | 6.963 | 1.818 |
| VH1.6/VL1.6 | 13.981 | 4.457 |
| VH1.7/VL1.4 | 22.624 | 13.241 |
| VH1.7/VL1.5 | 9.534 | 4.203 |
| VH1.7/vL1.6 | 4.637 | 1.422 |
| VH1.9/VL1.6 | 11.051 | 3.26 |
| VH1.12/VL1.8 | 16.259 | 5.843 |

TABLE 6
Steady State KD binding affinities for
monomeric Fabs to human and cyno CD47.

| | Cyno CD47 | | Human CD47 | |
|---|---|---|---|---|
| Analyte | KD (nM) | Chi2 | KD (nM) | Chi2 |
| FAB h5F9G4 | 17.1 | 0.00827 | 11.9 | 0.0265 |
| FAB VH1.8/1.1 | 17.9 | 0.0366 | 7.4 | 0.0126 |
| FAB VH1.8/1.6 | 25.9 | 0.0564 | 15.9 | 0.00446 |
| FAB VH1.8/1.6DI | 27.8 | 0.00741 | 20.3 | 0.00696 |

TABLE 7
Examples of antibody
variable region amino acid sequences.
VH1.8/VL1.6
heavy chain variable (VH) region
QVQLVQSGAEVKKPGASVKVSCKASGYTFTNYAMHWVRQAPGQRLEW
MGTIYPGNDDTKYNQKFQGRVTITADTSASTAYMELSSLRSEDTAVY
YCARGGFRAMDYWGQGTLVTVSS
(SEQ ID NO: 1)
light chain variable (VL) region
DIVMTQSPLSLPVTPGEPASISCRSSQSLLHSNANTYLGWYLQKPGQ
SPQLLIYKGSNRFSGVPDRFSGSGSGTDFTLKISRVEAEDVGVYYCF
QASHVPYTFGQGTKLEIK
(SEQ ID NO: 2)
VH1.8/VL1.6-DI
heavy chain variable (VH) region
QVQLVQSGAEVKKPGASVKVSCKASGYTFTNYAMHWVRQAPGQRLEW
MGTIYPGNDDTKYNQKFQGRVTITADTSASTAYMELSSLRSEDTAVY
YCARGGFRAMDYWGQGTLVTVSS
(SEQ ID NO: 3)
light chain variable (VL) region
DIVMTQSPLSLPVTPGEPASISCRSSQSLLHSNANTYLGWYLQKPGQ
SPQLAIYKGSNRFSGVPDRFSGSGSGTDFTLKISRVEAEDAGVYYCF
QASHVPYTFGQGTKLEIK
(SEQ ID NO: 4)
VH1.8/VL1.1
heavy chain variable (VH) region
QVQLVQSGAEVKKPGASVKVSCKASGYTFTNYAMHWVRQAPGQRLEW
MGTIYPGNDDTKYNQKFQGRVTITADTSASTAYMELSSLRSEDTAVY
YCARGGFRAMDYWGQGTLVTVSS
(SEQ ID NO: 5)
light chain variable (VL) region
DIVMTQSPLSLPVTPGEPASISCRSSQSLLHSNGNTYLGWYLQKPGQ
SPQLLIYKGSNRFSGVPDRFSGSGSGTDFTLKISRVEAEDVGVYYCF
QASHVPYTFGQGTKLEIK
(SEQ ID NO: 6)
VH1.8/VL1.5
heavy chain variable (VH) region
QVQLVQSGAEVKKPGASVKVSCKASGYTFTNYAMHWVRQAPGQRLEW
MGTIYPGNDDTKYNQKFQGRVTITADTSASTAYMELSSLRSEDTAVY
YCARGGFRAMDYWGQGTLVTVSS
(SEQ ID NO: 7)
light chain variable (VL) region
DIVMTQSPLSLPVTPGEPASISCRSSQSLLHSNAYNYLGWYLQKPGQ
SPQLLIYKGSNRFSGVPDRFSGSGSGTDFTLKISRVEAEDVGVYYCF
QASHVPYTFGQGTKLEIK
(SEQ ID NO: 8)
VH1.8/VL1.4
heavy chain variable (VH) region
QVQLVQSGAEVKKPGASVKVSCKASGYTFTNYAMHWVRQAPGQRLEW
MGTIYPGNDDTKYNQKFQGRVTITADTSASTAYMELSSLRSEDTAVY
YCARGGFRAMDYWGQGTLVTVSS
(SEQ ID NO: 9)
light chain variable (VL) region
DIVMTQSPLSLPVTPGEPASISCRSSQSLLHSNAYNYLGWYLQKPGQ
SPQLLIYKGSNRFSGVPDRFSGSGSGTDFTLKISRVEAEDVGVYYCF
QASQVPYTFGQGTKLEIK
(SEQ ID NO: 10)
VH1.8/VL1.3
heavy chain variable (VH) region
QVQLVQSGAEVKKPGASVKVSCKASGYTFTNYAMHWVRQAPGQRLEW
MGTIYPGNDDTKYNQKFQGRVTITADTSASTAYMELSSLRSEDTAVY
YCARGGFRAMDYWGQGTLVTVSS
(SEQ ID NO: 11)
light chain variable (VL) region
DIVMTQSPLSLPVTPGEPASISCRSSQSLLHSNANTYLGWYLQKPGQ
SPQLLIYKGSNRFSGVPDRFSGSGSGTDFTLKISRVEAEDVGVYYCF
QASQVPYTFGQGTKLEIK
(SEQ ID NO: 12)
VH1.8/VL1.2
heavy chain variable (VH) region
QVQLVQSGAEVKKPGASVKVSCKASGYTFTNYAMHWVRQAPGQRLEW
MGTIYPGNDDTKYNQKFQGRVTITADTSASTAYMELSSLRSEDTAVY
YCARGGFRAMDYWGQGTLVTVSS
(SEQ ID NO: 13)
light chain variable (VL) region
DIVMTQSPLSLPVTPGEPASISCRSSQSLLHSNGNTYLGWYLQKPGQ
SPQLLIYKGSNRFSGVPDRFSGSGSGTDFTLKISRVEAEDVGVYYCF
QASQVPYTFGQGTKLEIK
(SEQ ID NO: 14)
D6
heavy chain variable (VH) region
EVQLVQSGAEVKKPGESLKISCKGSGYSFTNYNIHWVRQMPGKGLEW
MGTIYPGNSDTSYNPSFQGQVTISADKSISTAYLQWSSLKASDTAMY
YCARGGVRAMDYWGQGTLVTVSS
(SEQ ID NO: 15)
light chain variable (VL) region
DIVMTQSPLSLPVTPGEPASISCRSSQSLLHSNGNTYLGWYLQKPGQ
SPQLLIYKGSNRFSGVPDRFSGSGSGTDFTLKISRVEAEDVGVYYCM
QASQVPYTFGQGTKLEIK
(SEQ ID NO: 16)

TABLE 8
Examples of antibody Fc region amino
acid sequences.

Human IgG4 wild type
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPSCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK (SEQ ID NO: 17)
Human IgG4(S228P)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK (SEQ ID NO: 18)
Human IgG1 wild type
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK (SEQ ID NO: 19)
Human IgG1-3M
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK (SEQ ID NO: 20)
Human IgG2 wild type
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDISVEWESNGQPENNYKTTPPMLDSDGSFFLYSKLTVDKSRWQQGN
VFSCSVMHEALHNHYTQKSLSLSPGK (SEQ ID NO: 21)
Human IgG1 wild type “REEM” allotype
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK (SEQ ID NO: 22)
Human IgG1-3M “REEM” allotype
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGAPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMKNQVSLTCL
VKGFYPSDIAVEWESNGQPENNYKTTPPVLDSDGSFFLYSKLTVDKSRWQ
QGNVFSCSVMHEALHNHYTQKSLSLSPGK (SEQ ID NO: 23)

TABLE 9
Examples of CD47 protein amino acid sequences.

Human CD47 sequence
MWPLVAALLLGSACCGSAQLLFNKTKSVEFTFCNDTVVIPCFVTNME
AQNTTEVYVKWKFKGRDIYTFDGALNKSTVPTDFSSAKIEVSQLLKG
DASLKMDKSDAVSHTGNYTCEVTELTREGETIIELKYRVVSWFSPNE
NILIVIFPIFAILLFWGQFGIKTLKYRSGGMDEKTIALLVAGLVITV
IVIVGAILFVPGEYSLKNATGLGLIVTSTGILILLHYYVFSTAIGLT
SFVIAILVIQVIAYILAVVGLSLCIAACIPMHGPLLISGLSILALAQ
LLGLVYMKFVASNQKTIQPPRKAVEEPLNAFKESKGMMNDE
(SEQ ID NO: 24)
Cynomolgus monkey CD47 sequence
MWPLVAALLLGSACCGSAQLLFNKTKSVEFTFCNDTVVIPCFVTNME
AQNTTEVYVKWKFKGRDIYTFDGALNKSTAPANFSSAKIEVSQLLKG
DASLKMDKSDAVSHTGNYTCEVTELTREGETIIELKYRVVSWFSPNE
NILIVIFPIFAILLFWGQFGIKTLKYRSGGMDEKTIALLVAGLMITV
IVIVGAILFVPGEYSLKNATGLGLIVTSTGILILLHYYVFSTAIGLT
SFVIAILVIQVIAYILAVVGLSLCIAACIPMHGPLLISGLSILALAQ
LLGLVYMKFVASNQKTIQPPRKAVEEPLNAFKESKGMMNDE
(SEQ ID NO: 25)

1.-43. (canceled)

44. An anti-CD47 antibody or an antigen-binding portion thereof, wherein the antibody comprises a heavy chain variable (VH) region and a light chain variable (VL) region, wherein:
(a) the VH region comprises a HCDR1 amino acid sequence of GYTFTNYAMH (SEQ ID NO: 48), a HCDR2 amino acid sequence of MGTIYPGNDDTKYNQKFQG (SEQ ID NO: 49), and a HCDR3 amino acid sequence of GGYRAEDY (SEQ ID NO:59);
and the VL region comprises a LCDR1 amino acid sequence of RSSQSLLHSNGNTYLG (SEQ ID NO:51), a LCDR2 amino acid sequence of KGSNRFS (SEQ ID NO:52), and a LCDR3 amino acid sequence of FQASHVPYT (SEQ ID NO:53);
(b) the VH region comprises a HCDR1 amino acid sequence of GYTFTNYAMH (SEQ ID NO: 48), a HCDR2 amino acid sequence of MGTIYPGNDDTKYNQKFQG (SEQ ID NO: 49), a HCDR3 amino acid sequence of GGYRAEDY (SEQ ID NO:59), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence FQASQVPYT (SEQ ID NO:54);
(c) the VH region comprises a HCDR1 amino acid sequence of GYTFTNYAMH (SEQ ID NO: 48), a HCDR2 amino acid sequence of MGTIYPGNDDTKYNQKFQG (SEQ ID NO: 49), a HCDR3 amino acid sequence of GGYRAEDY (SEQ ID NO:59), a LCDR1 amino acid sequence RSSQSLLHSNANTYLG (SEQ ID NO:55), a LCDR2 amino acid sequence of KGSNRFS (SEQ ID NO:52), a LCDR3 amino acid sequence FQASQVPYT (SEQ ID NO:54);
(d) the VH region comprises a HCDR1 amino acid sequence of GYTFTNYAMH (SEQ ID NO: 48), a HCDR2 amino acid sequence of MGTIYPGNDDTKYNQKFQG (SEQ ID NO: 49), a HCDR3 amino acid sequence of GGYRAEDY (SEQ ID NO:59), a LCDR1 amino acid sequence RSSQSLLHSNANTYLG (SEQ ID NO:55), a LCDR2 amino acid sequence of KGSNRFS (SEQ ID NO:52), a LCDR3 amino acid sequence FQASHVPYT (SEQ ID NO:53);
(e) the VH region comprises a HCDR1 amino acid sequence of GYTFTDYNMH (SEQ ID NO: 88), a HCDR2 amino acid sequence of MGTIYPGNDDTKYNQKFQG (SEQ ID NO: 49), a HCDR3 amino acid sequence of GGFRAMDY (SEQ ID NO:50), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence FQASHVPYT (SEQ ID NO:53);
(f) the VH region comprises a HCDR1 amino acid sequence of GYTFTNYNIH (SEQ ID NO: 89), a HCDR2 amino acid sequence of MGTIYPGNDDTSYSQKFQG (SEQ ID NO: 90), a HCDR3 amino acid sequence of GGFRAMDY (SEQ ID NO:50), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence FQASHVPYT (SEQ ID NO:53);
(g) the VH region comprises a HCDR1 amino acid sequence of GYTFTNYNIH (SEQ ID NO: 89), a HCDR2 amino acid sequence of MGTIYPGNDDTSYSQKFQG (SEQ ID NO: 90), a HCDR3 amino acid sequence of GGFRAMDY (SEQ ID NO:50), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence FQASQVPYT (SEQ ID NO:54);
(h) the VH region comprises a HCDR1 amino acid sequence of GYTFTNYNIH (SEQ ID NO: 89), a HCDR2 amino acid sequence of MGTIYPGNDDTSYSQKFQG (SEQ ID NO: 90), a HCDR3 amino acid sequence of GGFRAMDY (SEQ ID NO:50), a LCDR1 amino acid sequence RSSQSLLHSNANTYLG (SEQ ID NO:55), a LCDR2 amino acid sequence of KGSNRFS (SEQ ID NO:52), a LCDR3 amino acid sequence FQASQVPYT (SEQ ID NO:54);
(i) the VH region comprises a HCDR1 amino acid sequence of GYTFTNYNIH (SEQ ID NO: 89), a HCDR2 amino acid sequence of MGTIYPGNDDTSYSQKFQG (SEQ ID NO: 90), a HCDR3 amino acid sequence of GGFRAMDY (SEQ ID NO:50), a LCDR1 amino acid sequence RSSQSLLHSNAYNYLG (SEQ ID NO:56), a LCDR2 amino acid sequence of KGSNRFS (SEQ ID NO:52), a LCDR3 amino acid sequence FQASHVPYT (SEQ ID NO:53);
(j) the VH region comprises a HCDR1 amino acid sequence of GYTFTNYNIH (SEQ ID NO: 89), a HCDR2 amino acid sequence of MGTIYPGNDDTSYSQKFQG (SEQ ID NO: 90), a HCDR3 amino acid sequence of GGFRAMDY (SEQ ID NO:50), a LCDR1 amino acid sequence RSSQSLLHSNANTYLG (SEQ ID NO:55), a LCDR2 amino acid sequence of KGSNRFS (SEQ ID NO:52), a LCDR3 amino acid sequence of FQASHVPYT (SEQ ID NO:53);
(k) the VH region comprises a HCDR1 amino acid sequence of GYTFTNYAMH (SEQ ID NO: 48), a HCDR2 amino acid sequence of MGTIYPGNDDTSYSQKFQG (SEQ ID NO: 90), a HCDR3 amino acid sequence of GGYRAEDY (SEQ ID NO:59), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of FQASHVPYT (SEQ ID NO:53);
(1) the VH region comprises a HCDR1 amino acid sequence of GYTFTNYAMH (SEQ ID NO: 48), a HCDR2 amino acid sequence of MGTIYPGNDDTSYSQKFQG (SEQ ID NO: 90), a HCDR3 amino acid sequence of GGYRAEDY (SEQ ID NO:59), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of FQASQVPYT (SEQ ID NO:54);
(m) the VH region comprises a HCDR1 amino acid sequence of GYTFTNYAMH (SEQ ID NO: 48), a HCDR2 amino acid sequence of MGTIYPGNDDTSYSQKFQG (SEQ ID NO: 90), a HCDR3 amino acid sequence of GGYRAEDY (SEQ ID NO:59), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of FQASHVPYT (SEQ ID NO:53);
(n) the VH region comprises a HCDR1 amino acid sequence of GYTFTNYAMH (SEQ ID NO: 48), a HCDR2 amino acid sequence of MGTIYPGNDDTKYSQKFQG (SEQ ID NO: 91), a HCDR3 amino acid sequence of GGYRAEDY (SEQ ID NO:59), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of FQASQVPYT (SEQ ID NO:54);
(o) the VH region comprises a HCDR1 amino acid sequence GYSFTNYNIH (SEQ ID NO: 57), a HCDR2 amino acid sequence of MGTIYPGNSDTSYNPSFQG (SEQ ID NO: 61), a HCDR3 amino acid sequence of GGVRAMDY (SEQ ID NO:62), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of MQASQVPYT (SEQ ID NO:63);
(p) the VH region comprises a HCDR1 amino acid sequence GYSFTNYNIH (SEQ ID NO: 57), a HCDR2 amino acid sequence of MGTIYPGNSDTSYNPKFQG (SEQ ID NO: 92), a HCDR3 amino acid sequence of GGKRAMDY (SEQ ID NO:93), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of MQASQVPYT (SEQ ID NO:63);
(q) the VH region comprises a HCDR1 amino acid sequence GYSFTNYNIH (SEQ ID NO: 57), a HCDR2 amino acid sequence of MGTIYPGDSDTSYNPKFQG (SEQ ID NO: 94), a HCDR3 amino acid sequence of GGYRAQDY (SEQ ID NO:95), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of MQASHVPYT (SEQ ID NO:96);
(r) the VH region comprises a HCDR1 amino acid sequence GYSFTNYNIH (SEQ ID NO: 57), a HCDR2 amino acid sequence of MGTIYPGDSDTSYNPKFQG (SEQ ID NO: 94), a HCDR3 amino acid sequence of GGYRAEDY (SEQ ID NO:59), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of MQGSHVPYT (SEQ ID NO:97);
(s) the VH region comprises a HCDR1 amino acid sequence GYSFTNYNIH (SEQ ID NO: 57), a HCDR2 amino acid sequence of MGTIYPGDSDTRYNQKFQG (SEQ ID NO: 98), a HCDR3 amino acid sequence of GGIRAMDY (SEQ ID NO:99), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of FQGSHVPYI (SEQ ID NO:100);
(t) the VH region comprises a HCDR1 amino acid sequence GYSFTNYNIH (SEQ ID NO: 57), a HCDR2 amino acid sequence of MGTIYPGNSDTRYNPKFQG (SEQ ID NO: 101), a HCDR3 amino acid sequence of GGFRAMDY (SEQ ID NO:50), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of MQGSHVPYT (SEQ ID NO:97); or
(u) the VH region comprises a HCDR1 amino acid sequence GYSFTNYNIH (SEQ ID NO: 57), a HCDR2 amino acid sequence of MGTIYPGDSDTRYNPKFQG (SEQ ID NO: 58), a HCDR3 amino acid sequence of GGYRAEDY (SEQ ID NO:59), a LCDR1 amino acid sequence RSSQSLLHSNGNTYLG (SEQ ID NO:51), a LCDR2 amino acid sequence of KGSNRFS (SEQ ID NO:52), a LCDR3 amino acid sequence of MQGSHVPYT (SEQ ID NO:97).

45. The antibody or antigen-binding portion of claim 44, wherein the antibody or antigen-binding portion is humanized or chimeric.

46. The antibody or antigen-binding portion of claim 44, wherein the VH region, the VL region, or both the VH and the VL region comprise a human variable region framework scaffold amino acid sequence into which the CDRs have been inserted.

47. The antibody or antigen-binding portion of claim 44, wherein the VH region comprises an IGHV5-51 or IGHV1-3 human germline scaffold amino acid sequence into which the HCDR1, HCDR2 and HCDR3 amino acid sequences have been inserted.

48. The antibody or antigen-binding portion of claim 44, wherein the VL region comprises an IGKV2-28 human germline scaffold amino acid sequence into which the LCDR1, LCDR2 and LCDR3 amino acid sequences have been inserted.

49. The anti-CD47 antibody or antigen-binding portion of claim 47, wherein the VH region comprises an amino acid sequence having at least 90% sequence identity to:
(a) amino acid residues 1-25 of SEQ ID NO: 140;
(b) amino acid residues 35-47 of SEQ ID NO: 140;
(c) amino acid residues 67-98 of SEQ ID NO: 140; and
(d) amino acid residues 107-117 of SEQ ID NO: 140.

50. The anti-CD47 antibody or antigen-binding portion of claim 47, wherein the VH region comprises:
(a) amino acid residues 1-25 of SEQ ID NO: 140;
(b) amino acid residues 35-47 of SEQ ID NO: 140;
(c) amino acid residues 67-98 of SEQ ID NO: 140; and
(d) amino acid residues 107-117 of SEQ ID NO: 140.

51. The anti-CD47 antibody or antigen-binding portion of claim 48, wherein the VL region comprises an amino acid sequence having at least 90% sequence identity to:
(a) amino acid residues 24-39 of SEQ ID NO: 144;
(b) amino acid residues 40-54 of SEQ ID NO: 144;
(c) amino acid residues 62-93 of SEQ ID NO: 144; and
(d) amino acid residues 103-112 of SEQ ID NO: 144.

52. The anti-CD47 antibody or antigen-binding portion of claim 48, wherein the VL region comprises:
(a) amino acid residues 24-39 of SEQ ID NO: 144;
(b) amino acid residues 40-54 of SEQ ID NO: 144;
(c) amino acid residues 62-93 of SEQ ID NO: 144; and
(d) amino acid residues 103-112 of SEQ ID NO: 144.

53. The anti-CD47 antibody or antigen-binding portion of claim 44, wherein the antibody or antigen-binding portion comprises an immunoglobulin constant region.

54. The anti-CD47 antibody or antigen-binding portion of claim 54, wherein the immunoglobulin constant region is IgG, IgE, IgM, IgD, IgA, or IgY.

55. The anti-CD47 antibody or antigen-binding portion of claim 55, wherein the immunoglobulin constant region is IgG1, IgG2, IgG3, IgG4, IgA1, or IgA2.

56. The anti-CD47 antibody or antigen-binding portion of claim 54, wherein the immunoglobulin constant region is a wild-type human IgG4 constant region, a human IgG4 constant region comprising the amino acid substitution S228P, a wild-type human IgG1 constant region, a human IgG1 constant region comprising the amino acid substitutions L234A, L235A, and G237A, or a wild-type human IgG2 constant region.

57. The anti-CD47 antibody or antigen-binding portion of claim 54, wherein the immunoglobulin constant region comprises any one of SEQ ID NOs: 17-23.

58. The anti-CD47 antibody or antigen-binding portion of claim 44, wherein the antibody or antigen-binding portion is a Fab, a Fab′, a F(ab′)₂, a Fv, a scFv, a maxibody, a minibody, an intrabody, a diabody, a triabody, a tetrabody, or a bis-scFv.

59. The anti-CD47 antibody or antigen-binding portion of claim 44, wherein the antibody or antigen-binding portion is monoclonal.

60. The anti-CD47 antibody or antigen-binding portion of claim 44, wherein the antibody or antigen-binding portion is tetrameric, tetravalent, or multispecific.

61. A pharmaceutical composition comprising the antibody or antigen-binding portion of claim 44 and a pharmaceutically acceptable carrier.

62. A method of treating cancer, an autoimmune disease, an inflammatory disease, a cardiovascular disease or a fibrotic disease in a subject, comprising administering to the subject a therapeutically effective amount of the antibody or antigen-binding portion of claim 44.

63. The method of claim 62, wherein:
(a) the cancer is Gastrointestinal Stromal cancer (GIST), pancreatic cancer, melanoma, breast cancer, lung cancer, bronchial cancer, colorectal cancer, prostate cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, or cancer of the hematological tissues;
(b) the autoimmune disease or the inflammatory disease is arthritis, asthma, multiple sclerosis, psoriasis, Crohn's disease, inflammatory bowel disease, lupus, Grave's disease, Hashimoto's thyroiditis, or ankylosing spondylitis;
(c) the cardiovascular disease is coronary heart disease or atherosclerosis; or
(d) the fibrotic disease is myocardial infarction, angina, osteoarthritis, pulmonary fibrosis, cystic fibrosis, bronchitis, or asthma.
